SEQUENCE LISTING (1 sequence)

SEQ ID NO: 1 - protein, 247 aa, Artificial Sequence
  nanobody
EVQLVESGGGLVQAGGSLRLSCAASGGTFSFYGMGWFRQAPGKEQEFVADIRTSAGRTYYADSVKGRFTISRDNAKNTVYLQMNSLKPEDTAVYYCAAEMSGISGWDYWGQGTQVTVSSGGGGSGGGSEVQLVESGGGLVQAGGSLRLSCAASGGTFSFYGMGWFRQAPGKEQEFVADIRTSAGRTYYADSVKGRFTISRDNAKNTVYLQMNSLKPEDTAVYYCAAEMSGISGWDYWGQGTQVTVSS

(19) United States
(12) Patent Application Publication
(10) Pub. No.: US 2023/0110994 A1
(43) Pub. Date: Apr. 13, 2023

(54) CONJUGATES UNDERGOING INTRAMOLECULAR REARRANGEMENTS

(71) Applicant: Ascendis Pharma A/S, Hellerup (DK)

(72) Inventor: Samuel WEISBROD, Heidelberg (DE)

(21) Appl. No.: 17/790,589

(22) PCT Filed: Dec. 30, 2020
(86) PCT No.: PCT/EP2020/088034

§ 371 Date: Jul. 1, 2022

(30) Foreign Application Priority Data
Jan. 3, 2020 (EP) 20150243.2

(51) Int. Cl. A61K 47/68

(52) U.S. Cl. CPC A61K 47/6889; A61K 47/6803; A61K 47/6843

(57) ABSTRACT

The present invention relates to conjugates and pharmaceutically acceptable salts thereof, reagents, intermediates, methods for the synthesis of said conjugates, pharmaceutical compositions comprising said conjugates and the use of said conjugates.

The present invention relates to conjugates and pharmaceutically acceptable salts thereof, reagents, intermediates, methods for the synthesis of said conjugates, pharmaceutical compositions comprising said conjugates and the use of said conjugates.

To improve physicochemical or pharmacokinetic properties, such as the in vivo circulation half-life of drugs, such drugs can be conjugated to a carrier, such as a polymer. Typically, polymers in drug delivery are either used in a non-covalent complexation of the drug and polymer, embedding of drug in a polymer or by covalent conjugation of the drug to a polymeric moiety.

However, the non-covalent approach requires a highly efficient drug encapsulation to prevent uncontrolled, burst-type release of the drug due to the disintegration of the drug-polymer complex after administration. Restraining the diffusion of an unbound, water-soluble drug molecule requires strong van der Waals contacts, frequently mediated through hydrophobic moieties and charged moieties for electrostatic binding. Many conformationally sensitive drugs, such as proteins or peptides, are rendered dysfunctional during the complexation process and/or during subsequent storage of the non-covalently bound drug.

Alternatively, a drug may be covalently conjugated to a polymeric moiety via a linker moiety, whereby the linkage between the drug and the linker is stable or via a linker moiety, whereby the linkage between the drug and the linker moiety is reversible. If the drug is conjugated to the linker moiety via a stable linkage, such conjugate needs to exhibit sufficient residual activity to have a pharmaceutical effect and thus the conjugate is constantly in an active form.

The synthesis of conjugates comprising a drug that is covalently conjugated to the linker moiety via a reversible linkage, typically involves the reaction of a reagent comprising said linker moiety with a functional group on the drug moiety, such as an amine functional group. For example, WO2009/095479A2 discloses conjugates comprising a linker moiety that is conjugated to a drug via an amide bond, whereby said amide bond is rendered reversible for example by the neighboring group participation of functional groups or other groups that are comprised within the linker moiety, such as amine and amide groups. More specifically, a nucleophilic amine within the linker moiety enhances the nucleophilicity of the nitrogen atom comprised in an amide or thioamide, which in turn attacks the carbonyl moiety of the amide group that connects the drug to the linker moiety, resulting in the cleavage of the amide bond and the release of the drug in unmodified form. The synthesis of such conjugates may be challenging, as for example during the conjugation of the reagent comprising the linker moiety to the drug, the neighboring group participation effect has to be inactivated in order to avoid premature cyclization of the reagent comprising the linker moiety and the formation of side-products.

One way of avoiding said premature cyclization is by inactivating the nucleophilicity of one of the neighboring groups, such as of the amide group for example by the use of an amide protecting group. As such amide protecting group needs to be removed after the conjugation of the drug to the reagent comprising the linker moiety, such process synthesis may require the use of protecting group moieties that should be easily removed, such as under mild conditions. The choice of such protecting groups is limited for the skilled practitioner especially, when the drug moiety is a protein moiety, as for example the deprotection of said protecting group moieties has to occur preferably under aqueous conditions and with only limited use of organic solvents and reagents, to avoid inactivation or damaging of the protein.

Therefore, there is a need of identifying a solution to the challenges of chemical synthesis of conjugates comprising drug moieties, in particular protein drug moieties, that are linked to carriers via linker moieties and whereby the linkage between the drug and the linker is reversible.

It is thus an object of the present invention to at least partially overcome the shortcomings described above.

This object is achieved with a conjugate or a pharmaceutically acceptable salt thereof comprising at least one moiety -D conjugated via at least one moiety -L¹-L²- to at least one moiety Z, wherein a moiety -L¹- is conjugated to the nitrogen of a primary or secondary amine of a moiety -D and wherein the linkage between -D and -L¹- is reversible and wherein a moiety -L²- is conjugated to Z, wherein
each -D is independently a primary or secondary amine-comprising moiety of a drug D-H;
each -L²- is independently a single bond or a spacer moiety;
each Z is independently a polymeric moiety or a C₈₋₂₄ alkyl;
each -L¹- is independently a linker moiety of formula (I):

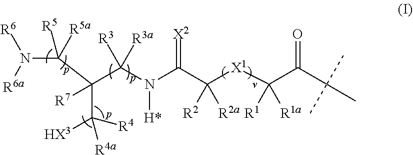

wherein
the dashed line indicates the attachment to the nitrogen of the primary or secondary amine of -D;
v is selected from the group consisting of 0 or 1;
—X¹— is selected from the group consisting of —C(R⁸)(R^{8a})—, —N(R⁹)— and —O—;
═X² is selected from the group consisting of ═O and ═N(R¹⁰);
—X³— is selected from the group consisting of —O—, —S— and —Se—;
each p is independently selected from the group consisting of 0 or 1, provided that at most one p is 0;
—R⁶, —R^{6a}, —R¹⁰ are independently selected from the group consisting of —H, —C(R¹¹)(R^{11a})(R^{11b}) and -T;
—R⁹ is selected from the group consisting of —C(R¹¹)(R^{11a})(R^{11b}) and -T;
—R¹, —R^{1a}, —R², —R^{2a}, —R³, —R^{3a}, —R⁴, —R^{4a}, —R⁵, —R^{5a}, —R⁷, —R⁸ —R^{8a}, —R¹¹, —R^{11a} and —R^{11b} are independently selected from the group consisting of —H, halogen, —CN, —C(O)OR¹², —OR¹², —C(O)R¹², —C(O)N(R¹²)(R^{12a}), —S(O)₂N(R¹²)(R^{12a}), —S(O)N(R¹²)(R^{12a}), —S(O)₂R¹², —S(O)R¹², —N(R¹²)S(O)₂N(R^{12a})(R^{12b}), —SR¹², —NO₂, —N(R¹²)C(O)OR^{12a}, —N(R¹²)C(O)N(R^{12a})(R^{12b}), —OC(O)N(R¹²)(R^{12a}), -T, C₁₋₆ alkyl, C₂₋₆ alkenyl and C₂₋₆ alkynyl; wherein C₁₋₆ alkyl, C₂₋₆ alkenyl and C₂₋₆ alkynyl are optionally substituted with one or more —R¹³, which are the same or different; and wherein C₁₋₆ alkyl, C₂₋₆ alkenyl and C₂₋₆ alkynyl are optionally interrupted by one or more groups selected from the group consisting of -T-, —C(O)O—, —O—, —C(O)—, —C(O)N(R¹⁴)—, —S(O)₂N(R¹⁴)—, —S(O)N(R¹⁴)—, —S(O)₂—, —S(O)—, —N(R¹⁴)S(O)₂N(R^{14a})—, —S—, —N(R¹⁴)—, —OC(OR¹⁴)(R^{14a})—, —N(R¹⁴)C(O)N(R^{14a})— and —OC(O)N(R¹⁴)—;
   —R¹², —R^{12a}, —R^{12b} are independently selected from the group consisting of —H, -T, C₁₋₆ alkyl, C₂₋₆ alkenyl and C₂₋₆ alkynyl; wherein -T, C₁₋₆ alkyl, C₂₋₆ alkenyl and C₂₋₆ alkynyl are optionally substituted with one or more —R¹³, which are the same or different and wherein C₁₋₆ alkyl, C₂₋₆ alkenyl and C₂₋₆ alkynyl are optionally interrupted by one or more groups selected from the group consisting of -T-, —C(O)O—, —O—, —C(O)—, —C(O)N(R¹⁴)—, —S(O)₂N(R¹⁴)—, —S(O)N(R¹⁴)—, —S(O)₂—, —S(O)—, —N(R¹⁴)S(O)₂N(R^{14a})—, —S—, —N(R¹⁴)—, —OC(OR¹⁴)(R^{14a})—, —N(R¹⁴)C(O)N(R^{14a})— and —OC(O)N(R¹⁴)—;
      wherein each T is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl; wherein each T is independently optionally substituted with one or more —R¹³, which are the same or different;
—R¹³ is selected from the group consisting of halogen, —CN, oxo, —C(O)OR¹⁵, —OR¹⁵, —C(O)R¹⁵, —C(O)N(R¹⁵)(R^{15a}), —S(O)₂N(R¹⁵)(R^{15a}), —S(O)N(R¹⁵)(R^{15a}), —S(O)₂R¹⁵, —S(O)R¹⁵, —N(R¹⁵)S(O)₂N(R^{15a})(R^{15b}), —SR¹⁵, —N(R¹⁵)(R^{15a}), —NO₂, —OC(O)R¹⁵, —N(R¹⁵)C(O)R^{15a}, —N(R¹⁵)S(O)₂R^{15a}, —N(R¹⁵)S(O)R^{15a}, —N(R¹⁵)C(O)OR^{15a}, —N(R¹⁵)C(O)N(R^{15a})(R^{15b}), —OC(O)N(R¹⁵)(R^{15a}) and C₁₋₆ alkyl; wherein C₁₋₆ alkyl is optionally substituted with one or more halogen, which are the same or different;
   wherein —R¹⁴, —R^{14a}, —R¹⁵, —R^{15a} and —R^{15b} are independently selected from the group consisting of —H and C₁₋₆ alkyl; wherein C₁₋₆ alkyl is optionally substituted with one or more halogen, which are the same or different;
optionally, one or more of the pairs —R¹/—R^{1a}, —R²/—R^{2a}, —R³/—R^{3a}, —R⁴/—R^{4a}, —R⁵/—R^{5a} or —R⁸/—R^{8a} are joined together with the atom to which they are attached to form a C₃₋₁₀ cycloalkyl, 3- to 10-membered heterocyclyl or an 8- to 11-membered heterobicyclyl;
optionally, one or more of the pairs —R¹/—R², —R¹/—R⁸, —R¹/—R⁹, —R²/—R⁹ or —R²/—R¹⁰ are joined together with the atoms to which they are attached to form a ring -A-;
   wherein -A- is selected from the group consisting of phenyl, naphthyl, indenyl, indanyl, tetralinyl, C₃₋₁₀ cycloalkyl, 3- to 10-membered heterocyclyl and 8- to 11-membered heterobicyclyl;
optionally, one or more of the pairs —R³/—R⁶, —R⁴/—R⁶, —R⁵/—R⁶, —R⁶/—R^{6a} or —R⁶/—R⁷ form together with the atoms to which they are attached a ring -A′-;
   wherein -A′- is selected from the group consisting of 3- to 10-membered heterocyclyl and 8- to 11-membered heterobicyclyl; and
each -L¹- is substituted with at least one -L²- and optionally further substituted provided that the hydrogen marked with the asterisk in formula (I) is not replaced by a substituent.

Another aspect of the present invention is a reagent comprising a moiety -L*-, wherein -L*- is conjugated to -Q, wherein
-Q is —OH or -LG, wherein -LG is a leaving group moiety;
-L*- is a linker moiety of formula (II)

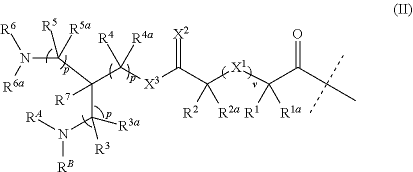

wherein
the dashed line indicates the attachment to -Q;
v is selected from the group consisting of 0 or 1;
—X¹— is selected from the group consisting of —C(R⁸)(R^{8a})—, —N(R⁹)— and —O—;
═X² is selected from the group consisting of ═O and ═N(R¹⁰);
—X³— is selected from the group consisting of —O—, —S— and —Se—;
each p is independently selected from the group consisting of 0 or 1, provided that at most one p is 0;
—R⁶ is —PG and —R^{6a} is selected from the group consisting of —H, —C(R¹¹)(R^{11a})(R^{11b}), -T and —PG; or —R⁶ and —R^{6a} are independently selected from the group consisting of —C(R¹¹)(R^{11a})(R^{11b}) and -T;
—R^{A} and —R^{B} are independently selected from the group consisting of —H and —PG provided that not more than one of —R^{A} or —R^{B} can be —H;
—PG is an amine protecting group moiety;
—R⁹ is selected from the group consisting of —C(R¹¹)(R^{11a})(R^{11b}) and -T;
—R¹⁰ is selected from the group consisting of —H, —C(R¹¹)(R^{11a})(R^{11b}) and -T;
—R¹, —R^{1a}, —R², —R^{2a}, —R³, —R^{3a}, —R⁴, —R^{4a}, —R⁵, —R^{5a}, —R⁷, —R⁸ —R^{8a}, —R¹¹, —R^{11a} and —R^{11b} are independently selected from the group consisting of —H, halogen, —CN, —C(O)OR¹², —OR¹², —C(O)R¹², —C(O)N(R¹²)(R^{12a}), —S(O)₂N(R¹²)(R^{12a}), —S(O)N(R¹²)(R^{12a}), —S(O)₂R¹², —S(O)R¹², —N(R¹²)S(O)₂N(R^{12a})(R^{12b}), —SR¹², —NO₂, —N(R¹²)C(O)OR^{12a}, —N(R¹²)C(O)N(R^{12a})(R^{12b}), —OC(O)N(R¹²)(R^{12a}), -T, C₁₋₆ alkyl, C₂₋₆ alkenyl and C₂₋₆ alkynyl; wherein C₁₋₆ alkyl, C₂₋₆ alkenyl and C₂₋₆ alkynyl are optionally substituted with one or more —R¹³, which are the same or different; and wherein C₁₋₆ alkyl, C₂₋₆ alkenyl and C₂₋₆ alkynyl are optionally interrupted by one or more groups selected from the group consisting of -T-, —C(O)O—, —O—, —C(O)—, —C(O)N(R¹⁴)—, —S(O)₂N(R¹⁴)—, —S(O)N(R¹⁴)—, —S(O)₂—, —S(O)—, —N(R¹⁴)S(O)₂N(R^{14a})—, —S—, —N(R¹⁴)—, —OC(OR¹⁴)(R^{14a})—, —N(R¹⁴)C(O)N(R^{14a})— and —OC(O)N(R¹⁴)—;
   —R¹², —R^{12a}, —R^{12b} are independently selected from the group consisting of —H, -T, C₁₋₆ alkyl, C₂₋₆ alkenyl, and C₂₋₆ alkynyl; wherein -T, C₁₋₆ alkyl, C₂₋₆ alkenyl, and C₂₋₆ alkynyl are optionally substituted with one or more —R¹³, which are the same or different and wherein C₁₋₆ alkyl, C₂₋₆ alkenyl, and C₂₋₆ alkynyl are optionally interrupted by one or more groups selected from the group consisting of -T-, —C(O)O—, —O—, —C(O)—, —C(O)N(R¹⁴)—, —S(O)₂N(R¹⁴)—, —S(O)N(R¹⁴)—, —S(O)₂—, —S(O)—, —N(R¹⁴)S(O)₂N(R^{14a})—, —S—, —N(R¹⁴)—, —OC(OR¹⁴)(R^{14a})—, —N(R¹⁴)C(O)N(R^{14a})— and —OC(O)N(R¹⁴)—;
      wherein each T is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl;
wherein each T is independently optionally substituted with one or more —R¹³, which are the same or different; —R¹³ is selected from the group consisting of halogen, —CN, oxo, —C(O)OR¹⁵, —OR¹⁵, —C(O)R¹⁵, —C(O)N(R¹⁵)(R^{15a}), —S(O)₂N(R¹⁵)(R^{15a}), —S(O)N(R¹⁵)(R^{15a}), —S(O)₂R¹⁵, —S(O)R¹⁵, —N(R¹⁵)S(O)₂N(R^{15a})(R^{15b}), —SR¹⁵, —N(R¹⁵)(R^{15a}), —NO₂, —OC(O)R¹⁵, —N(R¹⁵)C(O)R^{15a}, —N(R¹⁵)S(O)₂R^{15a}, —N(R¹⁵)S(O)R^{15a}, —N(R¹⁵)C(O)OR^{15a}, —N(R¹⁵)C(O)N(R^{15a})(R^{15b}), —OC(O)N(R¹⁵)(R^{15a}) and C₁₋₆ alkyl; wherein C₁₋₆ alkyl is optionally substituted with one or more halogen, which are the same or different;
   wherein —R¹⁴, —R^{14a}, —R¹⁵, —R^{15a} and —R^{15b} are independently selected from the group consisting of —H and C₁₋₆ alkyl; wherein C₁₋₆ alkyl is optionally substituted with one or more halogen, which are the same or different;
optionally, one or more of the pairs —R⁶/—R^{6a}, —R^{A}/—R^{B} or —R⁶/—R^{A} form a moiety —PG;
optionally, one or more of the pairs —R/—R^{1a}, —R²/—R^{2a}, —R³/—R^{3a}, —R⁴/—R^{4a}, —R⁵/—R^{5a} or —R⁸/—R^{8a} are joined together with the atom to which they are attached to form a C₃₋₁₀ cycloalkyl, 3- to 10-membered heterocyclyl or an 8- to 11-membered heterobicyclyl;
optionally, one or more of the pairs —R¹/—R², —R¹/—R⁸, —R¹/—R⁹, —R²/—R⁹ or —R²/—R¹⁰ are joined together with the atoms to which they are attached to form a ring -A-;
   wherein -A- is selected from the group consisting of phenyl, naphthyl, indenyl, indanyl, tetralinyl, C₃₋₁₀ cycloalkyl, 3- to 10-membered heterocyclyl and 8- to 11-membered heterobicyclyl;
optionally, one or more of the pairs —R³/—R⁶, —R⁴/—R⁶, —R⁵/—R⁶, —R⁶/—R^{6a} or —R⁶/—R⁷ form together with the atoms to which they are attached a ring -A′-;
   wherein -A′- is selected from the group consisting of 3- to 10-membered heterocyclyl and 8- to 11-membered heterobicyclyl;
wherein -L*- is optionally substituted with at least one moiety -L²-Z or at least one moiety -L²-Y and optionally is further substituted;
wherein -L²- is a single bond or a spacer moiety;
   Z is independently a polymeric moiety or a C₈₋₂₄ alkyl;
and wherein —Y is a functional group which may optionally be present in its protected form.

In certain embodiments, -L*- of formula (II) is substituted with at least one moiety -L²-Y or at least one moiety -L²-Z and optionally is further substituted. In certain embodiments, -L*- of formula (II) is substituted with at least one moiety -L²-Y or at least one moiety -L²-Z provided that —X³— is not —S— and optionally -L*- of formula (II) is further substituted.

In certain embodiments, -L*- of formula (II) is substituted with at least one moiety -L²-Y. In certain embodiments, -L*- of formula (II) is substituted with one moiety -L²-Y. In certain embodiments, -L*- of formula (II) is substituted with two moieties -L²-Y. In certain embodiments, -L*- of formula (II) is substituted with three moieties -L²-Y.

In certain embodiments, -L*- of formula (II) is substituted with at least one moiety -L²-Z. In certain embodiments, -L*- of formula (II) is substituted with one moiety -L²-Z. In certain embodiments, -L*- of formula (II) is substituted with two moieties -L²-Z. In certain embodiments, -L*- of formula (II) is substituted with three moieties -L²-Z.

It is understood that the phrase “optionally, the pair —R⁶/—R^{6a} forms a moiety —PG” in relation with a moiety of the structure

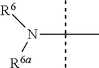

means that —R⁶ and —R^{6a} form together with the nitrogen atom to which they are attached an imine functional group such as

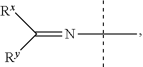

wherein —R^{x} and —R^{y} are independently selected from the group consisting of —H, C₁₋₄ alkyl, phenyl and methoxyphenyl;

or that —R⁶ and —R^{6a} form together with the nitrogen to which they are attached an azide functional group such as

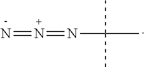

It is also understood that the phrase “optionally, the pair —R^{A}/—R^{B} can form a moiety —PG” in relation with a moiety of the structure

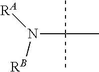

means that —R^{A} and —R^{B} form together with the nitrogen atom to which they are attached an imine functional group such as

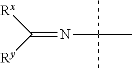

wherein —R^{x} and —R^{y} are independently selected from the group consisting of —H, C₁₋₄ alkyl, phenyl and methoxyphenyl;

or that —R^{A} and —R^{B} form together with the nitrogen to which they are attached an azide functional group such as

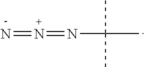

It is also understood that the phrase “optionally, the pair —R⁶/—R^{A} can form a moiety —PG” in relation with a moiety of the structure

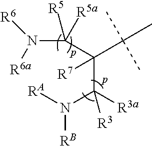

means that —R⁶ and —R^{A} form together with the nitrogen atoms to which they are attached a moiety

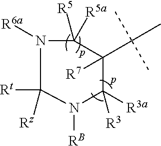

wherein —R^{t} and —R^{z} are independently selected from the group consisting of —H, C₁₋₄ alkyl, phenyl and methoxyphenyl and variables —R³, —R^{3a}, —R⁵, —R^{5a}, —R^{6a}, —R⁷, —R^{B} and p are defined as in formula (II).

Another aspect of the present invention is an intermediate (A) comprising a moiety -L*- of formula (II) as defined above for the reagent of the present invention, and wherein a moiety -L*- is conjugated to at least one moiety -D, wherein
each -D is independently a primary or secondary amine-comprising moiety of a drug D-H;
the dashed line in formula (II) indicates the attachment to the nitrogen of the primary or secondary amine of -D;
-L*- of formula (II) is optionally substituted with at least one moiety -L²-Z or at least one moiety -L²-Y and optionally is further substituted;
-L²- is independently a single bond or a spacer moiety;
Z is independently a polymeric moiety or a C₈₋₂₄ alkyl;
and wherein —Y is a functional group which may optionally be present in its protected form.

It is understood that the expression “—Y is a functional group which may optionally be present in its protected form” means that —Y may be reversibly connected to a protecting group moiety. An intermediate (A) comprising a linker moiety of formula -L*- may be obtained by reaction of a reagent comprising a linker moiety of formula -L*- with a drug D-H, such as by displacement of -Q. It is understood by the person skilled in the art that when -Q is —OH, the reaction of the reagent with the drug moiety D-H may take place in the presence of a coupling reagent, such as in the presence of a coupling reagent selected from the group consisting of (1-cyano-2-ethoxy-2-oxoethylidenaminooxy)dimethylamino-morpholino-carbenium, hexafluorophosphate, (1-[bis(dimethylamino)methylene]-1H-1,2,3-triazolo[4,5-b]pyridinium 3-oxide hexafluorophosphate, N,N,N,N′-tetramethyl-O-(1H-benzotriazol-1-yl)uronium hexafluorophosphate, O-(benzotriazol-1-yl)-N,N,N′,N′-tetramethyluronium, hexafluorophosphate, (benzotriazol-1-yloxy)tripyrrolidinophosphonium hexafluorophosphate and N,N,N′,N′-tetramethyl-O—(N-succinimidyl)uronium tetrafluoroborate.

Another aspect of the present invention is a method for synthesizing a conjugate or a pharmaceutically acceptable salt thereof as defined above. Conjugates or intermediates of the conjugates of the present invention may be prepared by known methods or in accordance with the method of synthesis described below.

A method for synthesizing a conjugate according to the present invention, wherein the method comprises the steps of
(a) providing a reagent comprising a linker -L*- of formula (II);
(b) conjugating the reagent of step (a) with a primary or secondary amine-comprising drug to obtain an intermediate (A);
(c) subjecting the intermediate (A) of step (b) to deprotection conditions to obtain an intermediate (C′) or conjugate comprising a linker -L¹- of formula (I) or an intermediate (B);
(d) optionally, subjecting the intermediate (B) or (C′) obtained from step (c) to shift conditions;
(e) optionally, deprotecting the intermediate (B) or (C′) of step (d); and (f) isolating the conjugate resulting from steps (c), (d) or (e);
wherein optionally at least one Z moiety is attached to at least one intermediate (A), (B) or (C′) in between steps (b) and (c), (c) and (d), (d) and (e) or (e) and (f).

It is understood that the intermediate (C′) of step (c) is conjugated to at least one Z moiety to result in a conjugate comprising a linker -L¹- of formula (I). In certain embodiments, intermediate (C′) of step (c) is conjugated to one Z moiety to result in a conjugate comprising a linker -L¹- of formula (I).

Another aspect is a method for synthesizing a conjugate according to the present invention, wherein the method comprises the steps of:
(a) providing a reagent comprising a linker -L*- of formula (II);
(b) conjugating the reagent of step (a) with a primary or secondary amine-comprising drug to obtain an intermediate (A);
(c) subjecting the intermediate (A) of step (b) to deprotection conditions to obtain a conjugate comprising a linker -L¹- of formula (I) or an intermediate (B);
(d) optionally, subjecting the intermediate (B) obtained from step (c) to shift conditions;
(e) optionally, deprotecting the intermediate (B) of step (d); and
(f) isolating the conjugate resulting from steps (c), (d) or (e);
wherein optionally at least one Z moiety is attached to at least one intermediate (A) or (B) in between steps (b) and (c), (c) and (d), (d) and (e) or (e) and (f).

It is understood that if a reagent comprising a linker -L*- of formula (II) is not substituted with at least one moiety -L²-Z or -L²-Y, then at least one Z moiety may be attached to intermediate (B) after step (d). In certain embodiments, one Z moiety is attached to intermediate (B) after step (d).

It is also understood that in the beforementioned method, the attachment or conjugation of at least one Z moiety to at least one intermediate (A) or (B) may be optional when the reagent in step (a) already comprises a linker -L*- of formula (II), which -L*- is already substituted with at least one moiety -L²-Z.

It is also clear to the skilled person that when —Y within for example a moiety -L*-Y, is present in its protected form, i.e. it is reversibly connected to a protecting group moiety, said moiety needs to be subjected to deprotection conditions before conjugation to Z.

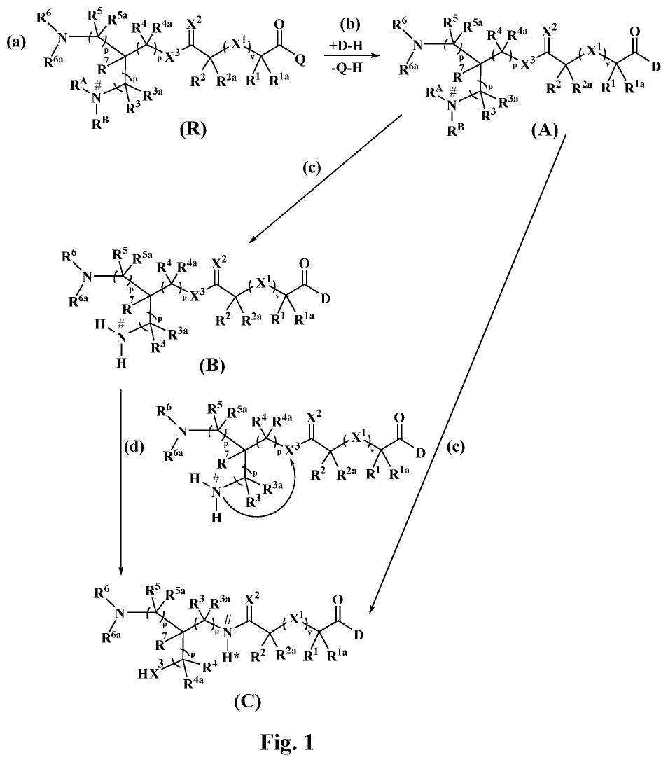

FIG. 1 shows an example of the rearrangements that take place during the steps of the method for synthesizing a conjugate according to the present invention, wherein the conjugate corresponds to structure (C) in FIG. 1. Step (a) involves the provision of a reagent comprising a linker -L*- of formula (II), which reagent corresponds to structure (R) in FIG. 1. Step (b) involves the conjugation of the reagent comprising a linker -L*- of formula (II), i.e. structure (R), with a primary or secondary amine-comprising drug, i.e. D-H and takes place by the nucleophilic attack of a primary or secondary amine functional group of the drug to the carbonyl group directly attached to the -Q moiety. This results in the release of Q-H and the formation of intermediate (A) which corresponds to structure (A) of FIG. 1. In step (c), intermediate (A) is subjected to deprotection conditions whereby variables —R^{A} and —R^{B} which are independently selected from the group consisting of —H and —PG, provided that not more than one —R^{A} or —RB can be —H, are converted into —H atoms and simultaneously the nitrogen atom marked with “#” undergoes an intramolecular shift with variable —X³— to result in a conjugate according to the present invention corresponding to structure (C) of FIG. 1. FIG. 1 also shows the optional situation whereby after intermediate (A) is subjected to deprotection conditions, variables —R^{A} and —R^{B} are converted into —H atoms to provide intermediate (B) corresponding to structure (B) of FIG. 1. In step (d) intermediate (B) obtained from step (c) is subjected to shift conditions under which the nitrogen atom marked with “#” undergoes an intramolecular shift with variable —X³— to result in a conjugate according to the present invention corresponding to structure (C). For simplification, no -L²-Z or -L²-Y moieties are shown. Also, for simplicity, optional step (e) is not shown in FIG. 1.

It is understood that in structures (R), (A) and (B) of FIG. 1, variables —R⁶ and —R^{6a} are defined as in formula (II) described above and if at least one of —R⁶ or —R^{6a} is —PG, then said variables may be converted into —H atoms under the conditions of steps (c) or (d), preferably under the conditions of step (e).

Within the meaning of the present invention the terms are used as follows.

As used herein, the term “drug” refers to a substance used in the treatment, cure, prevention or diagnosis of a disease or used to otherwise enhance physical or mental well-being of a patient. If a drug is conjugated to another moiety, the moiety of the resulting product that originated from the drug is referred to as “drug moiety”.

As used herein, the term “primary or secondary amine-comprising moiety of a drug D-H” refers to a moiety of a drug comprising at least one primary or secondary amine functional group, which drug may optionally have one or more further functional group(s) including one or more additional primary and/or secondary amine functional group(s).

As used herein, the term “moiety” means a part of a molecule, which lacks one or more atom(s) compared to the corresponding reagent. If, for example, a reagent of the formula “H—X—H” reacts with another reagent and becomes part of the reaction product, the corresponding moiety of the reaction product has the structure “H—X—” or “—X—”, whereas each “-” indicates attachment to another moiety. Accordingly, a drug moiety is released from a reversible linkage as a drug.

It is understood that if a sequence or chemical structure of a group of atoms is provided which group of atoms is attached to two moieties or is interrupting a moiety, said sequence or chemical structure can be attached to the two moieties in either orientation, unless explicitly stated otherwise. For example, a moiety “—C(O)N(R^{x})—” may be attached to two moieties or interrupting a moiety either as “—C(O)N(R^{x})—” or as “—N(R^{x})C(O)—”.

As used herein, the term “protecting group moiety” refers to a moiety which is reversibly connected to a functional group to render it incapable of reacting with, for example, another functional group. Suitable alcohol (—OH) protecting groups are, for example, acetyl, benzoyl, benzyl, β-methoxyethoxymethyl ether, dimethoxytrityl, methoxymethyl ether, methoxytrityl, p-methoxybenzyl ether, methylthiomethyl ether, pivaloyl, tetrahydropyranyl, trityl, trimethylsilyl, tert-butyldimethylsilyl, tri-iso-propylsilyloxymethyl, triisopropylsilyl ether, methyl ether, and ethoxyethyl ether. Suitable carbonyl protecting groups are, for example, acetals and ketals, acylals and dithianes. Suitable carboxylic acid protecting groups are, for example, methyl esters, benzyl esters, tert-butyl esters, 2,6-dimethylphenol, 2,6-diisopropylphenol, 2,6.-di-tert-butylphenol, silyl esters, orthoesters, and oxazoline. Suitable phosphate protecting groups are, for example, 2-cyanoethyl and methyl.

As used herein, the term “amine protecting group moiety” refers to a moiety that is used for the reversible protection of an amine functional group during chemical reaction processes to render said amine incapable of reacting with, for example, another functional group.

As used herein, the term “leaving group moiety” refers to an atom or group of atoms that is detached from the rest of a molecule, such as from a reagent, during a chemical reaction with another functional group.

As used herein, the term “deprotection conditions” refer to conditions under which at least one protecting group moiety, such as an amine protecting group moiety of an intermediate, is detached or cleaved from the functional group, such as from an amine group, such as under conditions that may involve the use of an acid, base, reducing agent, oxidizing agent, hydrogenation or light and optionally a scavenger reagent.

As used herein, the term “reducing agent” refers to a chemical compound or element that loses or donates an electron to an electron recipient such as an oxidizing agent in a redox chemical reaction.

As used herein, the term “oxidizing agent” refers to a chemical compound that is able to oxidize other chemical compounds.

As used herein, the term “scavenger reagent” refers to a chemical compound that traps another reaction intermediate, such as a reactive reaction intermediate.

As used herein, the term “shift conditions” refer to conditions under which a primary amine of an intermediate, such as intermediate (B), may intramolecularly rearrange, such as under conditions that may involve the use of a buffering agent or an organic solvent.

As used herein, the term “buffer” or “buffering agent” refers to a chemical compound that maintains the pH in a desired range. Physiologically tolerated buffers are, for example acetate, adipate, alanine, ammonium, arginine, ascorbate, aspartate, benzoate, bicarbonate, carbonate, citrate, diethanolamine, edetate, ethylenediamine, fumarate, gluconate, glutamate, glycine, guanidine, histidine, lactate, lysine, malate, metaphosphate, pentetate, phosphate, pyruvate, sorbate, succinate, tartrate, tromethamine and α-ketoglutarate.

As used herein, the term “polar protic solvent” refers to a solvent which comprises bonds between atoms with different electronegativities, has large dipole moments and has at least one hydrogen atom directly bound to an electronegative atom such as an oxygen, nitrogen or sulfur atom.

As used herein, the term “polar aprotic solvent” refers to a solvent which comprises bonds between atoms with different electronegativities, has large dipole moments and does not have a hydrogen atom directly bound to an electronegative atom such as an oxygen, nitrogen or sulfur atom.

As used herein, the term “reagent” means a chemical compound, which comprises at least one functional group for reaction with the functional group of another chemical compound or drug. It is understood that a drug comprising a functional group is also a reagent.

It is recognized by one of ordinary skill in the art that the conjugates of the present invention are prodrugs. As used herein, the term “prodrug” refers to a drug moiety, that is reversibly and covalently conjugated to a fatty acid-derived moiety or to a polymeric moiety, such as Z, through at least one -L¹-L²- moiety. A prodrug releases the reversibly and covalently bound drug moiety -D in the form of its corresponding drug D-H. In other words, a prodrug is a conjugate comprising a drug moiety, which is covalently and reversibly conjugated to a polymeric moiety via at least one -L¹-L²- moiety. Such prodrugs or conjugates release the formerly conjugated drug moiety in the form of a free drug.

As used herein, the term “reversible linkage” or “biodegradable linkage” is a linkage that is cleavable, in the absence of enzymes under physiological conditions, which are aqueous buffer at pH 7.4 and 37° C., with a half-life ranging from one hour to six months, such as from ten hours to four months, such as from one day to three months, from two days to two months or from three days to one month. It is understood, however, that a reversible linkage may also be cleavable at other conditions, such as for example at a different pH or at a different temperature with a half-life ranging from one hour to six months, but that a test for determining reversibility is performed in the above-described physiological conditions (aqueous buffer, pH 7.4, 37° C.). Accordingly, a “stable linkage” is a linkage having a half-life under physiological conditions of more than six months.

As used herein, the term “about” in combination with a numerical value is used to indicate a range ranging from and including the numerical value plus and minus no more than 10% of said numerical value, in certain embodiments, no more than 8% of said numerical value, in certain embodiments, no more than 5% of said numerical value and in certain embodiments, no more than 2% of said numerical value. For example, the phrase “about 200” is used to mean a range ranging from and including 200+/−10%, i.e. ranging from and including 180 to 220; in certain embodiments, 200+/−8%, i.e. ranging from and including 184 to 216; in certain embodiments, ranging from and including 200+/−5%, i.e. ranging from and including 190 to 210; and in certain embodiments 200+/−2%, i.e. ranging from and including 196 to 204. It is understood that a percentage given as “about 20%” does not mean “20%+/−10%”, i.e. ranging from and including 10 to 30%, but “about 20%” means ranging from and including 18 to 22%, i.e. plus and minus 10% of the numerical value which is 20.

As used herein, the term “C₁₋₄ alkyl” alone or in combination means a straight-chain or branched alkyl moiety having 1 to 4 carbon atoms. If present at the end of a molecule, examples of straight-chain or branched C₁₋₄ alkyl are methyl, ethyl, n-propyl, isopropyl, n-butyl, isobutyl, sec-butyl and tert-butyl. When two moieties of a molecule are linked by the C₁₋₄ alkyl, then examples for such C₁₋₄ alkyl groups are —CH₂—, —CH₂—CH₂—, —CH(CH₃)—, —CH₂—CH₂—CH₂—, —CH(C₂H₅)—, —C(CH₃)₂—. Each hydrogen of a C₁₋₄ alkyl carbon may optionally be replaced by a substituent as defined below. Optionally, a C₁₋₄ alkyl may be interrupted by one or more moieties as defined below.

As used herein, the term “C₁₋₆ alkyl” alone or in combination means a straight-chain or branched alkyl moiety having 1 to 6 carbon atoms. If present at the end of a molecule, examples of straight-chain and branched C₁₋₆ alkyl groups are methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl. When two moieties of a molecule are linked by the C₁₋₆ alkyl group, then examples for such C₁₋₆ alkyl groups are —CH₂—, —CH₂—CH₂—, —CH(CH₃)—, —CH₂—CH₂—CH₂—, —CH(C₂H₅)— and —C(CH₃)₂—. Each hydrogen atom of a C₁₋₆ carbon may optionally be replaced by a substituent as defined below. Optionally, a C₁₋₆ alkyl may be interrupted by one or more moieties as defined below.

Accordingly, “C₁₋₁₀ alkyl”, “C₁₋₂₀ alkyl”, “C₈₋₂₄ alkyl” or “C₁₋₅₀ alkyl” means an alkyl chain having 1 to 10, 1 to 20, 8 to 24 or 1 to 50 carbon atoms, respectively, wherein each hydrogen atom of the C₁₋₁₀, C₁₋₂₀, C₈₋₂₄ or C₁₋₅₀ carbon may optionally be replaced by a substituent as defined below. Optionally, a C₁₋₁₀ alkyl, C₁₋₂₀ alkyl, C₈₋₂₄ alkyl or C₁₋₅₀ alkyl may be interrupted by one or more moieties as defined below.

As used herein, the term “C₂₋₆ alkenyl” alone or in combination means a straight-chain or branched hydrocarbon moiety comprising at least one carbon-carbon double bond having 2 to 6 carbon atoms. If present at the end of a molecule, examples are —CH═CH₂, —CH═CH—CH₃, —CH₂—CH═CH₂, —CH═CHCH₂—CH₃ and —CH═CH—CH═CH₂. When two moieties of a molecule are linked by the C₂₋₆ alkenyl group, then an example of such C₂₋₆ alkenyl is —CH═CH—. Each hydrogen atom of a C₂₋₆ alkenyl moiety may optionally be replaced by a substituent as defined below. Optionally, a C₂₋₆ alkenyl may be interrupted by one or more moieties as defined below.

Accordingly, the terms “C₂₋₁₀ alkenyl”, “C₂₋₂₀ alkenyl” or “C₂₋₅₀ alkenyl” alone or in combination mean a straight-chain or branched hydrocarbon moiety comprising at least one carbon-carbon double bond having 2 to 10, 2 to 20 or 2 to 50 carbon atoms, respectively. Each hydrogen atom of a C₂₋₁₀ alkenyl, C₂₋₂₀ alkenyl or C₂₋₅₀ alkenyl group may optionally be replaced by a substituent as defined below. Optionally, a C₂₋₁₀ alkenyl, C₂₋₂₀ alkenyl or C₂₋₅₀ alkenyl may be interrupted by one or more moieties as defined below.

As used herein, the term “C₂₋₆ alkynyl” alone or in combination means a straight-chain or branched hydrocarbon moiety comprising at least one carbon-carbon triple bond having 2 to 6 carbon atoms. If present at the end of a molecule, examples are —C≡CH, —CH₂—C≡CH, CH₂—CH₂—C≡CH and CH₂—C≡C—CH₃. When two moieties of a molecule are linked by the alkynyl group, then an example is —C≡C—. Each hydrogen atom of a C₂₋₆ alkynyl group may optionally be replaced by a substituent as defined below. Optionally, one or more double bond(s) may occur. Optionally, a C₂₋₆ alkynyl may be interrupted by one or more moieties as defined below.

Accordingly, as used herein, the term “C₂₋₁₀ alkynyl”, “C₂₋₂₀ alkynyl” and “C₂₋₅₀ alkynyl” alone or in combination means a straight-chain or branched hydrocarbon moiety comprising at least one carbon-carbon triple bond having 2 to 10, 2 to 20 or 2 to 50 carbon atoms, respectively. Each hydrogen atom of a C₂₋₁₀ alkynyl, C₂₋₂₀ alkynyl or C₂₋₅₀ alkynyl group may optionally be replaced by a substituent as defined below. Optionally, one or more double bond(s) may occur. Optionally, a C₂₋₁₀ alkynyl, C₂₋₂₀ alkynyl or C₂₋₅₀ alkynyl may be interrupted by one or more moieties as defined below.

As mentioned above, a C₁₋₄ alkyl, C₁₋₆ alkyl, C₁₋₁₀ alkyl, C₁₋₂₀ alkyl, C₁₋₅₀ alkyl, C₈₋₂₄ alkyl, C₂₋₆ alkenyl, C₂₋₁₀ alkenyl, C₂₋₂₀ alkenyl, C₂₋₅₀ alkenyl, C₂₋₆ alkynyl, C₂₋₁₀ alkynyl, C₂₋₂₀ alkenyl or C₂₋₅₀ alkynyl may optionally be interrupted by one or more moieties which in certain embodiments are selected from the group consisting of

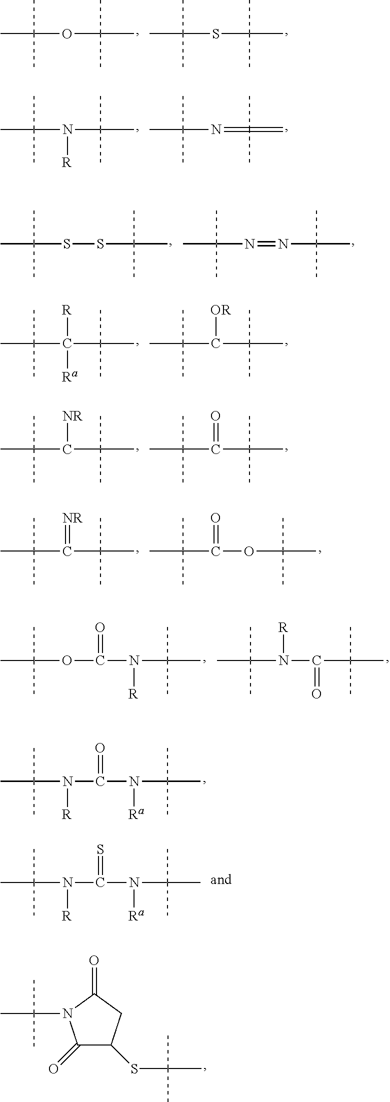

wherein
dashed lines indicate attachment to the remainder of the moiety or reagent; —R and —R^{a} are independently selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl; and which moieties and linkages are optionally further substituted.

As used herein, the term “C₃₋₁₀ cycloalkyl” means a cyclic alkyl chain having 3 to 10 carbon atoms, which may be saturated or unsaturated, e.g. cyclopropyl, cyclobutyl, cyclopentyl, cyclohexyl, cyclohexenyl, cycloheptyl, cyclooctyl, cyclononyl or cyclodecyl. Each hydrogen atom of a C₃₋₁₀ cycloalkyl carbon may be replaced by a substituent as defined below. The term “C₃₋₁₀ cycloalkyl” also includes bridged bicycles like norbornane or norbornene.

As used herein, the term “8- to 30-membered carbopolycyclyl” or “8- to 30-membered carbopolycycle” means a cyclic moiety of two or more rings with 8 to 30 ring atoms, where two neighboring rings share at least one ring atom and that may contain up to the maximum number of double bonds (aromatic or non-aromatic ring which is fully, partially or un-saturated). In certain embodiments, an 8- to 30-membered carbopolycyclyl means a cyclic moiety of two, three, four or five rings. In certain embodiments, an 8- to 30-membered carbopolycyclyl means a cyclic moiety of two, three or four rings.

As used herein, the term “3- to 10-membered heterocyclyl” or “3- to 10-membered heterocycle” means a ring with 3, 4, 5, 6, 7, 8, 9 or 10 ring atoms that may contain up to the maximum number of double bonds (aromatic or non-aromatic ring which is fully, partially or un-saturated) wherein at least one ring atom up to 4 ring atoms are replaced by a heteroatom selected from the group consisting of sulfur (including —S(O)—, —S(O)₂—), oxygen and nitrogen (including ═N(O)—) and wherein the ring is linked to the rest of the molecule via a carbon or nitrogen atom.

Examples for 3- to 10-membered heterocycles include but are not limited to aziridine, oxirane, thiirane, azirine, oxirene, thiirene, azetidine, oxetane, thietane, furan, thiophene, pyrrole, pyrroline, imidazole, imidazoline, pyrazole, pyrazoline, oxazole, oxazoline, isoxazole, isoxazoline, thiazole, thiazoline, isothiazole, isothiazoline, thiadiazole, thiadiazoline, tetrahydrofuran, tetrahydrothiophene, pyrrolidine, imidazolidine, pyrazolidine, oxazolidine, isoxazolidine, thiazolidine, isothiazolidine, thiadiazolidine, sulfolane, pyran, dihydropyran, tetrahydropyran, imidazolidine, pyridine, pyridazine, pyrazine, pyrimidine, piperazine, piperidine, morpholine, tetrazole, triazole, triazolidine, tetrazolidine, diazepane, azepine and homopiperazine. Each hydrogen atom of a 3- to 10-membered heterocyclyl or 3- to 10-membered heterocyclic group may be replaced by a substituent as defined below.

As used herein, the term “8- to 11-membered heterobicyclyl” or “8- to 11-membered heterobicycle” means a heterocyclic moiety of two rings with 8 to 11 ring atoms, where at least one ring atom is shared by both rings and that may contain up to the maximum number of double bonds (aromatic or non-aromatic ring which is fully, partially or un-saturated) wherein at least one ring atom up to 6 ring atoms are replaced by a heteroatom selected from the group consisting of sulfur (including —S(O)—, —S(O)₂—), oxygen and nitrogen (including ═N(O)—) and wherein the ring is linked to the rest of the molecule via a carbon or nitrogen atom. Examples for an 8- to 11-membered heterobicycle are indole, indoline, benzofuran, benzothiophene, benzoxazole, benzisoxazole, benzothiazole, benzisothiazole, benzimidazole, benzimidazoline, quinoline, quinazoline, dihydroquinazoline, quinoline, dihydroquinoline, tetrahydroquinoline, decahydroquinoline, isoquinoline, decahydroisoquinoline, tetrahydroisoquinoline, dihydroisoquinoline, benzazepine, purine and pteridine. The term 8- to 11-membered heterobicycle also includes spiro structures of two rings like 1,4-dioxa-8-azaspiro[4.5]decane or bridged heterocycles like 8-aza-bicyclo[3.2.1]octane. Each hydrogen atom of an 8- to 11-membered heterobicyclyl or 8- to 11-membered heterobicycle carbon may be replaced by a substituent as defined below.

Similarly, the term “8- to 30-membered heteropolycyclyl” or “8- to 30-membered heteropolycycle” means a heterocyclic moiety of more than two rings with 8 to 30 ring atoms, in certain embodiments of three, four or five rings, where two neighboring rings share at least one ring atom and that may contain up to the maximum number of double bonds (aromatic or non-aromatic ring which is fully, partially or unsaturated), wherein at least one ring atom up to 10 ring atoms are replaced by a heteroatom selected from the group of sulfur (including —S(O)—, —S(O)₂—), oxygen and nitrogen (including ═N(O)—) and wherein the ring is linked to the rest of a molecule via a carbon or nitrogen atom.

It is understood that the phrase “the pair —R^{x}/—R^{y} is joined together with the atom to which they are attached to form a C₃₋₁₀ cycloalkyl, 3- to 10-membered heterocyclyl or an 8- to 11-membered heterobicyclyl” in relation with a moiety of the structure:

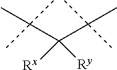

means that —R^{x} and —R^{y} form the following structure:

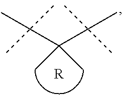

wherein R is C₃₋₁₀ cycloalkyl, 3- to 10-membered heterocyclyl or an 8- to 11-membered heterobicyclyl.

It is also understood that the phrase “the pair —R^{x}/—R^{y} is joined together with the atoms to which they are attached to form a ring -A-” in relation with a moiety of the structure:

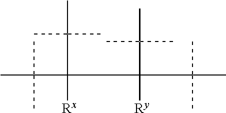

means that —R^{x} and —R^{y} form the following structure:

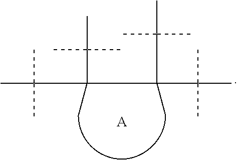

As used herein, the term “excipient” refers to a diluent, adjuvant or vehicle with which the therapeutic, such as a drug or conjugate, is administered. Such pharmaceutical excipient can be sterile liquids, such as water and oils, including those of petroleum, animal, vegetable or synthetic origin, including peanut oil, soybean oil, mineral oil and sesame oil. Water is a preferred excipient when the pharmaceutical composition is administered orally. Saline and aqueous dextrose are preferred excipients when the pharmaceutical composition is administered intravenously. Saline solutions and aqueous dextrose and glycerol solutions are preferably employed as liquid excipients for injectable solutions. Suitable pharmaceutical excipients include starch, glucose, lactose, sucrose, mannitol, trehalose, gelatin, malt, rice, flour, chalk, silica gel, sodium stearate, glycerol monostearate, talc, sodium chloride, dried skim milk, glycerol, propylene, glycol, hyaluronic acid, propylene glycol, water and ethanol. The pharmaceutical composition, if desired, can also contain minor amounts of wetting or emulsifying agents, pH buffering agents, like, for example, acetate, succinate, tris, carbonate, phosphate, HEPES (4-(2-hydroxyethyl)-1-piperazineethanesulfonic acid), MES (2-(N-morpholino)ethanesulfonic acid) or can contain detergents, like Tween®, poloxamers, poloxamines, CHAPS, Igepal® or amino acids like, for example, glycine, lysine or histidine. These pharmaceutical compositions can take the form of solutions, suspensions, emulsions, tablets, pills, capsules, powders or sustained-release formulations. The pharmaceutical composition can be formulated as a suppository, with traditional binders and excipients such as triglycerides. Oral formulation can include standard excipients such as pharmaceutical grades of mannitol, lactose, starch, magnesium stearate, sodium saccharine, cellulose, magnesium carbonate, etc. Such compositions will contain a therapeutically effective amount of the drug or drug moiety, together with a suitable amount of excipient so as to provide the form for proper administration to the patient. The formulation should suit the mode of administration.

As used herein, the term “free form” of a drug refers to the drug in its unmodified, pharmacologically active form, e.g. after being released from the conjugate.

As used herein, the term “functional group” means a group of atoms which can react with other groups of atoms. Exemplary functional groups are carboxylic acid, primary amine, secondary amine, tertiary amine, maleimide, thiol, sulfonic acid, carbonate, carbamate, hydroxyl, aldehyde, ketone, hydrazine, isocyanate, isothiocyanate, phosphoric acid, phosphonic acid, haloacetyl, alkyl halide, acryloyl, aryl fluoride, hydroxylamine, disulfide, sulfonamides, sulfuric acid, vinyl sulfone, vinyl ketone, diazoalkane, oxirane and aziridine.

As used herein, the term “halogen” means fluoro, chloro, bromo or iodo. In certain embodiments, halogen is fluoro or chloro.

As used herein, the term “interrupted” means that a moiety is inserted in between two carbon atoms or—if the insertion is at one of the moiety's ends—between a carbon or heteroatom and a hydrogen atom, in certain embodiments between a carbon and a hydrogen atom.

In case the conjugates of the present invention comprise one or more acidic or basic groups, the invention also comprises their corresponding pharmaceutically or toxicologically acceptable salts, in particular their pharmaceutically utilizable salts. Thus, the conjugates of the present invention comprising acidic groups can be used according to the invention, for example, as alkali metal salts, alkaline earth metal salts or as ammonium salts. More precise examples of such salts include sodium salts, potassium salts, calcium salts, magnesium salts or salts with ammonia or organic amines such as, for example, ethylamine, ethanolamine, triethanolamine or amino acids, or quaternary ammoniums, such as tetrabutylammonium and cetyl trimethylammonium. Conjugates of the present invention comprising one or more basic groups, i.e. groups which can be protonated, can be present and can be used according to the invention in the form of their addition salts with inorganic or organic acids. Examples for suitable acids include hydrogen chloride, hydrogen bromide, phosphoric acid, sulfuric acid, nitric acid, methanesulfonic acid, p-toluenesulfonic acid, naphthalenedisulfonic acids, oxalic acid, acetic acid, tartaric acid, lactic acid, salicylic acid, benzoic acid, formic acid, propionic acid, pivalic acid, diethylacetic acid, malonic acid, succinic acid, pimelic acid, fumaric acid, maleic acid, malic acid, sulfaminic acid, phenylpropionic acid, gluconic acid, ascorbic acid, isonicotinic acid, citric acid, adipic acid, trifluoroacetic acid and other acids known to the person skilled in the art. For the person skilled in the art further methods are known for converting the basic group into a cation like the alkylation of an amine group resulting in a positively-charged ammonium group and an appropriate counterion of the salt. If the conjugates of the present invention simultaneously comprise acidic and basic groups, the invention also includes, in addition to the salt forms mentioned, inner salts or betaines (zwitterions). The respective salts can be obtained by customary methods, which are known to the person skilled in the art like, for example by contacting these prodrugs with an organic or inorganic acid or base in a solvent or dispersant, or by anion exchange or cation exchange with other salts. The present invention also includes all salts of the conjugates of the present invention which, owing to low physiological compatibility, are not directly suitable for use in pharmaceuticals but which can be used, for example, as intermediates for chemical reactions or for the preparation of pharmaceutically acceptable salts.

As used herein, the term “pharmaceutically acceptable” means a substance that does not cause harm when administered to a patient and preferably means approved by a regulatory agency, such as the EMA (Europe) and/or the FDA (US) and/or any other national regulatory agency for use in animals, preferably for use in humans.

As used herein, the term “peptide” as used herein refers to a chain of at least 2 and up to and including 50 amino acid monomer moieties, which may also be referred to as “amino acid residues”, linked by peptide (amide) linkages. The amino acid monomers may be selected from the group consisting of proteinogenic amino acids and non-proteinogenic amino acids and may be D- or L-amino acids. The term “peptide” also includes peptidomimetics, such as peptoids, beta-peptides, cyclic peptides and depsipeptides and covers such peptidomimetic chains with up to and including 50 monomer moieties. The cyclic peptides may be mono-, bi-, tri- or tetracyclic peptides. The term “peptide” also includes lasso peptides.

As used herein, the term “protein” refers to a chain of more than 50 amino acid monomer moieties, which may also be referred to as “amino acid residues”, linked by peptide linkages, in which preferably no more than 12000 amino acid monomers are linked by peptide linkages, such as no more than 10000 amino acid monomer moieties, no more than 8000 amino acid monomer moieties, no more than 5000 amino acid monomer moieties or no more than 2000 amino acid monomer moieties.

As used herein, the term “small molecule drug” refers to drugs that are organic compounds with a molecular weight of less than 1000 Da, such as less than 900 Da or less than 800 Da. It is understood that nucleobase-based drug moieties, such as adenine or guanine analogues, may also be a type of small molecule drugs.

As used herein, the term “medium molecule drug” refers to drugs that are organic compounds which are not peptides and which are not proteins, and have a molecular weight ranging from and including 1 kDa to 7.5 kDa.

As used herein, the term “oligonucleotide” refers to double- or single-stranded RNA and DNA with preferably 2 to 1000 nucleotides and any modifications thereof. Modifications include for example, those which provide other chemical groups that incorporate additional charge, polarizability, hydrogen bonding, electrostatic interaction, and fluxionality to the nucleic acid ligand bases or to the nucleic acid ligand as a whole. Such modifications include for example, to 2′-position sugar modifications, 5-position pyrimidine modifications, 8-position purine modifications, modifications at exocyclic amines, substitution of 4-thiouridines, substitution of 5-bromo or 5-iodo-uracil; backbone modifications, methylations, unusual base-pairing combinations such as the isobases isocytidine and isoguanidine. Modifications can also include 3′ and 5′ modifications such as capping and change of stereochemistry. The term also includes aptamers.

As used herein, the term “peptide nucleic acids” refers to organic polymers having a peptidic backbone, i.e. a backbone in which the monomers are connected to each other through peptide linkages, to which nucleobases such as adenine, cytosine, guanine, thymine and uracil, are attached. In certain embodiments, the peptide backbone comprises N-(2-aminoethyl)-glycine.

As used herein, the term “polymer” means a molecule comprising repeating structural units, i.e. the monomers, connected by chemical bonds in a linear, circular, branched, crosslinked or dendrimeric way or a combination thereof, which may be of synthetic or biological origin or a combination of both. The monomers may be identical, in which case the polymer is a homopolymer, or may be different, in which case the polymer is a heteropolymer. A heteropolymer may also be referred to as a “copolymer” and includes for example alternating copolymers in which monomers of different types alternate; periodic copolymers in which monomers of different types of monomers are arranged in a repeating sequence; statistical copolymers in which monomers of different types are arranged randomly; block copolymers in which blocks of different homopolymers consisting of only one type of monomers are linked by a covalent bond; and gradient copolymers in which the composition of different monomers changes gradually along a polymer chain. It is understood that a polymer may also comprise one or more other moieties, such as, for example, one or more functional groups. Likewise, it is understood that also a peptide or protein is a polymer, even though the side chains of individual amino acid residues may be different. It is understood that for covalently crosslinked polymers, such as hydrogels, no meaningful molecular weight ranges can be provided.

As used herein, the term “polymeric” or “polymeric moiety” refers to a reagent or a moiety comprising one or more polymers or polymer moieties. A polymeric reagent or moiety may optionally also comprise one or more other moiety/moieties, which in certain embodiments are selected from the group consisting of:
C₁₋₅₀ alkyl, C₂₋₅₀ alkenyl, C₂₋₅₀ alkynyl, C₃₋₁₀ cycloalkyl, 3- to 10-membered heterocyclyl, 8- to 11-membered heterobicyclyl, phenyl, naphthyl, indenyl, indanyl and tetralinyl;
branching points, such as —CR<, >C< or —N<; and
linkages selected from the group comprising

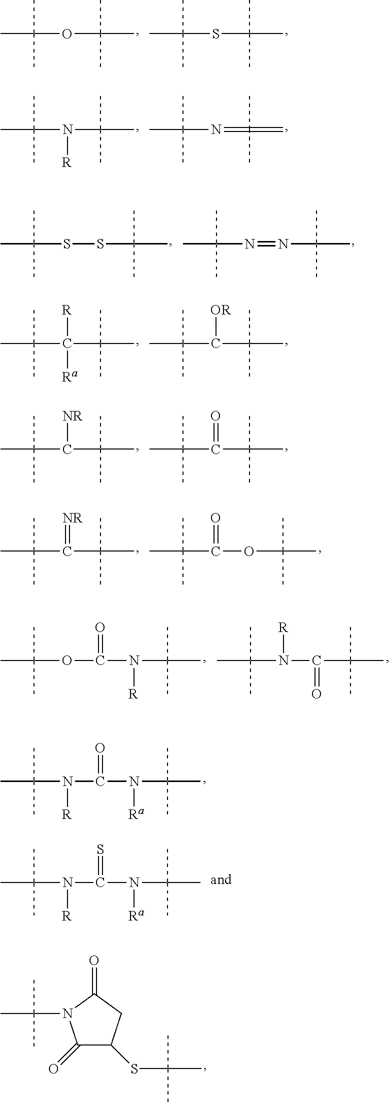

wherein
dashed lines indicate attachment to the remainder of the moiety or reagent; —R and —R^{a} are independently selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl; and which moieties and linkages are optionally further substituted.

The person skilled in the art understands that the polymerization products obtained from a polymerization reaction do not all have the same molecular weight, but rather exhibit a molecular weight distribution. Consequently, the molecular weight ranges, molecular weights, ranges of numbers of monomers in a polymer and numbers of monomers in a polymer as used herein, refer to the number average molecular weight and number average of monomers, i.e. to the arithmetic mean of the molecular weight of the polymer or polymeric moiety and the arithmetic mean of the number of monomers of the polymer or polymeric moiety.

Accordingly, in a polymeric moiety comprising “x” monomer units any integer given for “x” therefore corresponds to the arithmetic mean number of monomers. Any range of integers given for “x” provides the range of integers in which the arithmetic mean numbers of monomers lies.

An integer for “x” given as “about x” means that the arithmetic mean numbers of monomers lies in a range of integers of x+/−10%, in certain embodiments lies in a range of integers x+/−8%, in certain embodiments lies in a range of integers x+/−5% and in certain embodiments lies in a range of integers x+/−2%.

As used herein, the term “number average molecular weight” means the ordinary arithmetic mean of the molecular weights of the individual polymers.

As used herein, the term “PEG-based” in relation to a moiety or reagent means that said moiety or reagent comprises PEG. In certain embodiments, such PEG-based moiety or reagent comprises at least 10% (w/w) PEG, such as at least 20% (w/w) PEG, such as at least 30% (w/w) PEG, such as at least 40% (w/w) PEG, such as at least 50% (w/w), such as at least 60% (w/w) PEG, such as at least 70% (w/w) PEG, such as at least 80% (w/w) PEG, such as at least 90% (w/w) PEG, such as at least 95% (w/w) PEG. The remaining weight percentage of the PEG-based moiety or reagent may be other moieties, such as those selected from the group consisting of:
C₁₋₅₀ alkyl, C₂₋₅₀ alkenyl, C₂₋₅₀ alkynyl, C₃₋₁₀ cycloalkyl, 3- to 10-membered heterocyclyl, 8- to 11-membered heterobicyclyl, phenyl, naphthyl, indenyl, indanyl and tetralinyl;
branching points, such as —CR<, >C< or —N<; and
linkages selected from the group comprising

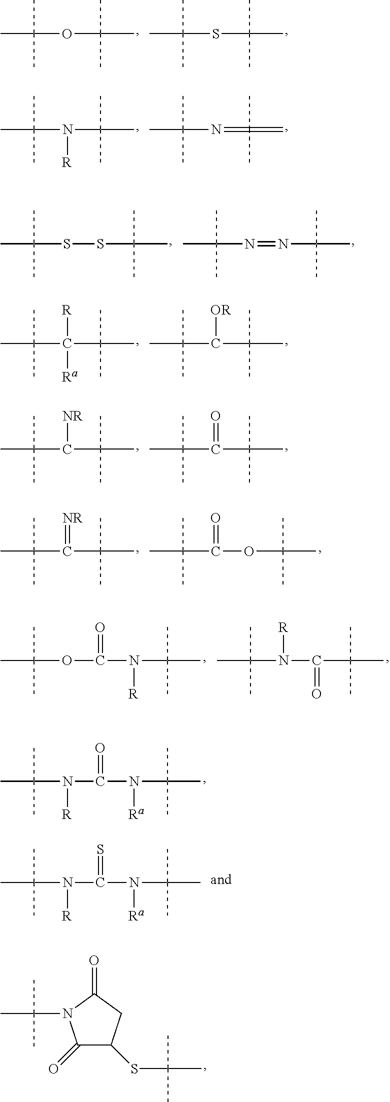

wherein
dashed lines indicate attachment to the remainder of the moiety or reagent;
—R and —R^{a} are independently selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl; and which moieties and linkages are optionally further substituted.

As used herein, the term “PEG-based comprising at least X % PEG” in relation to a moiety or reagent means that said moiety or reagent comprises at least X % (w/w) ethylene glycol units (—CH₂CH₂O—), wherein the ethylene glycol units may be arranged blockwise, alternating or may be randomly distributed within the moiety or reagent. In certain embodiments, all ethylene glycol units of said moiety or reagent are present in one block; the remaining weight percentage of the PEG-based moiety or reagent are other moieties in certain embodiments selected from the group consisting of:
C₁₋₅₀ alkyl, C₂₋₅₀ alkenyl, C₂₋₅₀ alkynyl, C₃₋₁₀ cycloalkyl, 3- to 10-membered heterocyclyl, 8- to 11-membered heterobicyclyl, phenyl, naphthyl, indenyl, indanyl, and tetralinyl;
branching points, such as —CR<, >C< or —N<; and
linkages selected from the group comprising

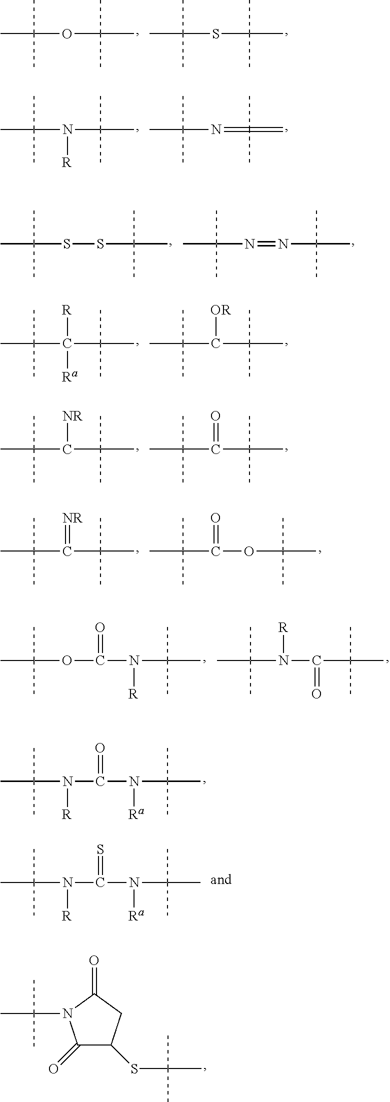

wherein
dashed lines indicate attachment to the remainder of the moiety or reagent, and wherein —R and —R^{a} are independently selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl; and which moieties and linkages are optionally further substituted.

As used herein, the term “hyaluronic acid-based” in relation to a moiety or reagent means that said moiety or reagent comprises hyaluronic acid. Such hyaluronic acid-based moiety or reagent comprises at least 10% (w/w) hyaluronic acid, such as at least 20% (w/w) hyaluronic acid, such as at least 30% (w/w) hyaluronic acid, such as at least 40% (w/w) hyaluronic acid, such as at least 50% (w/w) hyaluronic acid, such as at least 60 (w/w) hyaluronic acid, such as at least 70% (w/w) hyaluronic acid, such as at least 80% (w/w) hyaluronic acid, such as at least 90% (w/w) hyaluronic acid, or such as at least 95% (w/w) hyaluronic acid. The remaining weight percentage of the hyaluronic acid-based moiety or reagent may be other moieties, such as those selected from the group consisting of:
C₁₋₅₀ alkyl, C₂₋₅₀ alkenyl, C₂₋₅₀ alkynyl, C₃₋₁₀ cycloalkyl, 3- to 10-membered heterocyclyl, 8- to 11-membered heterobicyclyl, phenyl, naphthyl, indenyl, indanyl, and tetralinyl;
branching points, such as —CR<, >C< or —N<; and
linkages selected from the group consisting of

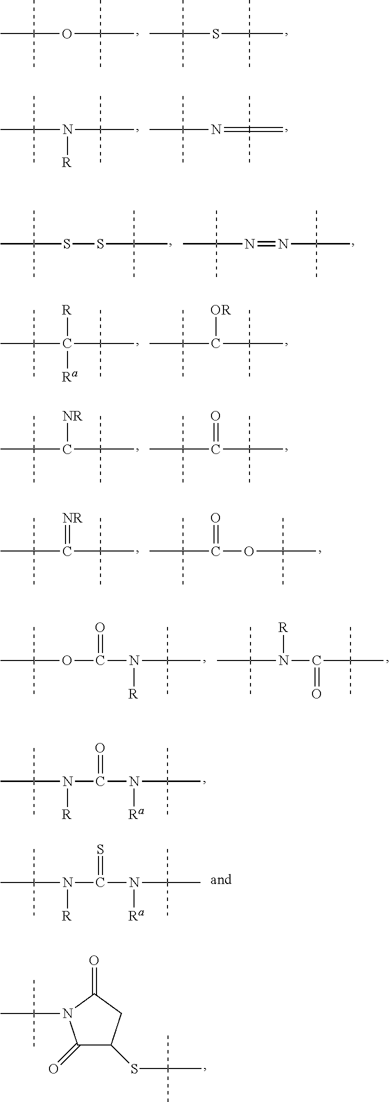

wherein
dashed lines indicate attachment to the remainder of the moiety or reagent; —R and —R^{a} are independently selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl; and which moieties and linkages are optionally further substituted.

As used herein, the term “hydrogel” means a hydrophilic or amphiphilic polymeric network composed of homopolymers or copolymers, which is insoluble due to the presence of hydrophobic interactions, hydrogen bonds, ionic interactions and/or covalent chemical crosslinks. The crosslinks provide the network structure and physical integrity.

As used herein, the term “random coil” refers to a peptide or protein adopting/having/forming, in certain embodiments having, a conformation which substantially lacks a defined secondary and tertiary structure as determined by circular dichroism spectroscopy performed in aqueous buffer at ambient temperature, and pH 7.4. In certain embodiments, the ambient temperature is about 20° C., i.e. between 18° C. and 22° C., while in certain embodiments the ambient temperature is 20° C.

As used herein, the term “spacer” or “spacer moiety” refers to a moiety suitable for connecting two moieties. Suitable spacers may be selected from the group consisting of C₁₋₅₀ alkyl, C₂₋₅₀ alkenyl and C₂₋₅₀ alkynyl, which C₁₋₅₀ alkyl, C₂₋₅₀ alkenyl or C₂₋₅₀ alkynyl is optionally interrupted by one or more groups selected from —NH—, —N(C₁₋₄ alkyl)-, —O—, —S—, —C(O)—, —C(O)NH—, —C(O)N(C₁₋₄ alkyl)-, —O—C(O)—, —S(O)—, —S(O)₂—, 4- to 7-membered heterocyclyl, phenyl and naphthyl and may optionally be substituted.

As used herein, the term “substituted” means that one or more —H atom(s) of a molecule or moiety are replaced by a different atom or a group of atoms, which are referred to as “substituent”.

As used herein, the term “substituent” refers in certain embodiments to a moiety selected from the group consisting of halogen, —CN, —C(O)OR^{x1}, —OR^{x1}, —C(O)R^{x1}, —C(O)N(R^{x1})(R^{x1a}), —S(O)₂N(R^{x1})(R^{x1a}), —S(O)N(R^{x1})(R^{x1a}), —S(O)₂R^{x1}, —S(O)R^{x1}, —N(R^{x1})S(O)₂N(R^{x1a})(R^{x1b}), —SR^{x1}, —N(R^{x1})(R^{x1a}), —NO₂, —OC(O)R^{x1}, —N(R^{x1})C(O)R^{x1a}, —N(R^{x1})S(O)₂R^{x1a}, —N(R^{x1})S(O)R^{x1a}, —N(R^{x1})C(O)OR^{x1a}, —N(R^{x1})C(O)N(R^{x1a})(R^{x1b}), —OC(O)N(R^{x1})(R^{x1a}), -T⁰, C₁₋₅₀ alkyl, C₂₋₅₀ alkenyl and C₂₋₅₀ alkynyl; wherein -T⁰, C₁₋₅₀ alkyl, C₂₋₅₀ alkenyl and C₂₋₅₀ alkynyl are optionally substituted with one or more —R^{x2}, which are the same or different and wherein C₁₋₅₀ alkyl, C₂₋₅₀ alkenyl and C₂₋₅₀ alkynyl are optionally interrupted by one or more groups selected from the group consisting of -T⁰-, —C(O)O—, —O—, —C(O)—, —C(O)N(R^{x3})—, —S(O)₂N^{x3})—, —S(O)N(R^{x3}), —S(O)₂, —S(O)—, —N(R^{x3})S(O)₂N(R^{x3a})—, —S—, —N(R^{x3})—, —OC(OR^{x3})(R^{x3a})—, —N(R^{x3})C(O)N(R^{x3a})— and —OC(O)N(R^{x3})—;

—R^{x1}, —R^{x1a}, —R^{x1b} are independently selected from the group consisting of —H, -T⁰, C₁₋₅₀ alkyl, C₂₋₅₀ alkenyl and C₂₋₅₀ alkynyl; wherein -T⁰, C₁₋₅₀ alkyl, C₂₋₅₀ alkenyl and C₂₋₅₀ alkynyl are optionally substituted with one or more —R^{x2}, which are the same or different and wherein C₁₋₅₀ alkyl, C₂₋₅₀ alkenyl and C₂₋₅₀ alkynyl are optionally interrupted by one or more groups selected from the group consisting of -T⁰-, —C(O)O—, —O—, —C(O)—, —C(O)N(R^{x3})—, —S(O)₂N(R^{x3})—, —S(O)N(R^{x3})—, —S(O)₂—, —S(O)—, —N(R^{x3})S(O)₂N(R^{x3a})—, —S—, —N(R^{x3})—, —OC(OR^{x3})(R^{x3a})—, —N(R^{x3})C(O)N(R^{x3a})— and —OC(O)N(R^{x3})—;

each T⁰ is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl; wherein each T⁰ is independently optionally substituted with one or more —R^{x2} which are the same or different;

each —R^{x2} is independently selected from the group consisting of halogen, —CN, oxo (═O), —C(O)OR^{x4}, —OR^{x4}, —C(O)R^{x4}, —C(O)N(R^{x4})(R^{x4a}), —S(O)₂N(R^{x4})(R^{x4a}), —S(O)N(R^{x4})(R^{x4a}), —S(O)₂R^{x4}, —S(O)R^{x4}, —N(R^{x4})S(O)₂N(R^{x4a})(R^{x4b}), —SR^{x4}, —N(R^{x4})(R^{x4a}), —NO₂, —OC(O)R^{x4}, —N(R^{x4})C(O)R^{x4a}, —N(R^{x4})S(O)₂R^{x4a}, —N(R^{x4})S(O)R^{x4a}, —N(R^{x4})C(O)OR^{x4a}, —N(R^{x4})C(O)N(R^{x4a})(R^{x4b}), —OC(O)N(R^{x4})(R^{x4a}) and C₁₋₆ alkyl; wherein C₁₋₆ alkyl is optionally substituted with one or more halogen, which are the same or different;

each —R^{x3}, —R^{x3a}, —R^{x4}, —R^{x4a}, —R^{x4b} is independently selected from the group consisting of —H and C₁₋₆ alkyl; wherein C₁₋₆ alkyl is optionally substituted with one or more halogen, which are the same or different.

In certain embodiments, the term “substituent” refers to a moiety selected from the group consisting of halogen, —CN, —C(O)OR^{x1}, —OR^{x1}, —C(O)R^{x1}, —C(O)N(R^{x1})(R^{x1a}), —S(O)₂N(R^{x1})(R^{x1a}), —S(O)N(R^{x1})(R^{x1a}), —S(O)₂R^{x1}, —S(O)R^{x1}, —N(R^{x1})S(O)₂N(R^{x1})(R^{x1a}), —SR^{x1}, —N(R^{x1})(R^{x1a}), —NO₂, —OC(O)R^{x1}, —N(R^{x1})C(O)R^{x1a}, —N(R^{x1})S(O)₂R^{x1a}, —N(R^{x1})S(O)R^{x1a}, —N(R^{x1})C(O)OR^{x1a}, —N(R^{x1})C(O)N(R^{x1})(R^{x1a}), —OC(O)N(R^{x1})(R^{x1a}), -T⁰, C₁₋₁₀ alkyl, C₂₋₁₀ alkenyl, and C₂₋₁₀ alkynyl; wherein -T⁰, C₁₋₁₀ alkyl, C₂₋₁₀ alkenyl, and C₂₋₁₀ alkynyl are optionally substituted with one or more —R^{x2}, which are the same or different and wherein C₁₋₁₀ alkyl, C₂₋₁₀ alkenyl, and C₂₋₁₀ alkynyl are optionally interrupted by one or more groups selected from the group consisting of -T⁰-, —C(O)O—, —O—, —C(O)—, —C(O)N(R^{x3})—, —S(O)₂N(R^{x3})—, —S(O)N(R^{x3})—, —S(O)₂—, —S(O)—, —N(R^{x3})S(O)₂N(R^{x3a})—, —S—, —N(R^{x3})—, —OC(OR^{x3})(R^{x3a})—, —N(R^{x3})C(O)N(R^{x3a})— and —OC(O)N(R^{x3})—;
each —R^{x1}, —R^{x1a}, —R^{x1b}, —R^{x3}, —R^{x3a} is independently selected from the group consisting of —H, halogen, C₁₋₆ alkyl, C₂₋₆ alkenyl and C₂₋₆ alkynyl;
each T⁰ is independently selected from the group consisting of phenyl, naphthyl, indenyl, indanyl, tetralinyl, C₃₋₁₀ cycloalkyl, 3- to 10-membered heterocyclyl, and 8- to 11-membered heterobicyclyl; wherein each T⁰ is independently optionally substituted with one or more —R^{x2} which are the same or different;
each —R^{x2} is independently selected from the group consisting of halogen, —CN, oxo (═O), —C(O)OR^{x4}, —OR^{x4}, —C(O)R^{x4}, —C(O)N(R^{x4})(R^{x4a}), —S(O)₂N(R^{x4})(R^{x4a}), —S(O)N(R^{x4})(R^{x4a}), —S(O)₂R^{x4}, —S(O)R^{x4}, —N(R^{x4})S(O)₂N(R^{x4a})(R^{x4b}), —SR^{x4}, —N(R^{x4})(R^{x4a}), —NO₂, —OC(O)R^{x4}, —N(R^{x4})C(O)R^{x4a}, —N(R^{x4})S(O)₂R^{x4a}, —N(R^{x4})S(O)R^{x4a}, —N(R^{x4})C(O)OR^{x4a}, —N(R^{x4})C(O)N(R^{x4a})(R^{x4b}), —OC(O)N(R^{x4})(R^{x4a}) and C₁₋₆ alkyl; wherein C₁₋₆ alkyl is optionally substituted with one or more halogen, which are the same or different;
each —R^{x4}, —R^{x4a}, —R^{x4b} is independently selected from the group consisting of —H, halogen, C₁₋₆ alkyl, C₂₋₆ alkenyl, and C₂₋₆ alkynyl.

In certain embodiments, the term “substituent” refers to a moiety selected from the group consisting of halogen, —CN, —C(O)OR^{x1}, —OR^{x1}, —C(O)R^{x1}, —C(O)N(R^{x1})(R^{x1a}), —S(O)₂N(R^{x1})(R^{x1a}), —S(O)N(R^{x1})(R^{x1a}), —S(O)₂R^{x1}, —S(O)R^{x1}, —N(R^{x1})S(O)₂N(R^{x1a})(R^{x1b}), —SR^{x1}, —N(R^{x1})(R^{x1a}), —NO₂, —OC(O)R^{x1}, —N(R^{x1})C(O)R^{x1a}, —N(R^{x1})S(O)₂R^{x1a}, —N(R^{x1})S(O)R^{x1a}, —N(R^{x1})C(O)OR^{x1a}, —N(R^{x1})C(O)N(R^{x1a})(R^{x1b}), —OC(O)N(R^{x1})(R^{x1a}), -T⁰, C₁₋₆ alkyl, C₂₋₆ alkenyl, and C₂₋₆ alkynyl; wherein -T⁰, C₁₋₆ alkyl, C₂₋₆ alkenyl, and C₂₋₆ alkynyl are optionally substituted with one or more —R^{x2}, which are the same or different and wherein C₁₋₆ alkyl, C₂₋₆ alkenyl, and C₂₋₆ alkynyl are optionally interrupted by one or more groups selected from the group consisting of -T⁰-, —C(O)O—, —O—, —C(O)—, —C(O)N(R^{x3})—, —S(O)₂N(R^{x3})—, —S(O)N(R^{x3})—, —S(O)₂—, —S(O)—, —N(R^{x3})S(O)₂N(R^{x3a})—, —S—, —N(R^{x3})—, —OC(OR^{x3})(R^{x3a})—, —N(R^{x3})C(O)N(R^{x3a})—, and —OC(O)N(R^{x3})—; each —R^{x1}, —R^{x1a}, —R^{x1b}, —R^{x2}, —R^{x3}, —R^{x3a} is independently selected from the group consisting of —H, halogen, C₁₋₆ alkyl, C₂₋₆ alkenyl, and C₂₋₆ alkynyl;

each T⁰ is independently selected from the group consisting of phenyl, naphthyl, indenyl, indanyl, tetralinyl, C₃₋₁₀ cycloalkyl, 3- to 10-membered heterocyclyl, and 8- to 11-membered heterobicyclyl; wherein each T⁰ is independently optionally substituted with one or more —R^{x2} which are the same or different.

In certain embodiments, a maximum of 6 —H atoms of an optionally substituted molecule are independently replaced by a substituent, e.g. 5 —H atoms are independently replaced by a substituent, 4 —H atoms are independently replaced by a substituent, 3 —H atoms are independently replaced by a substituent, 2 —H atoms are independently replaced by a substituent, or 1 —H atom is replaced by a substituent.

As used herein, the term “therapeutically effective amount” means an amount sufficient to cure, alleviate or partially arrest the clinical manifestations of a given disease and its complications. Effective amounts for each purpose will depend on the severity of the disease or injury as well as the weight and general state of the subject.

As used herein, the term “water-insoluble” refers to a compound of which less than 1 g can be dissolved in one liter of water at 20° C. to form a homogeneous solution. Accordingly, the term “water-soluble” refers to a compound of which 1 g or more can be dissolved in one liter of water at 20° C. to form a homogeneous solution.

In general, the term “comprise(s)” or “comprising” also encompasses “consist of” or “consisting of”.

In certain embodiments, all moieties -D of the conjugate are identical, i.e. have the same chemical structure. In such case all moieties -D of the conjugate derive from the same type of drug molecule.

In certain embodiments, the conjugate of the present invention comprises different moieties -D, i.e. comprises moieties -D with different chemical structures. These different structures derive from different types of drug molecules. In certain embodiments, the conjugate of the present invention comprises two different types of moieties -D. In certain embodiments, the conjugate of the present invention comprises three different types of moieties -D. In certain embodiments, the conjugate of the present invention comprises four different types of moieties -D. In certain embodiments, the conjugate of the present invention comprises five different types of moieties -D.

If the conjugates of the present invention comprise more than one type of -D, all moieties -D may be conjugated to the same type of -L¹- or may be conjugated to different types of -L¹-, i.e. a first type of -D may be conjugated to a first type of -L¹-, a second type of -D may be conjugated to a second type of -L¹- and so on. Using different types of -L¹- may, in certain embodiments, allow different release kinetics for different types of -D, such as for example a faster release for a first type of -D, a medium release for a second type of -D and a slow release for a third type of -D. Accordingly, in certain embodiments the conjugates of the present invention comprise one type of -L¹-. In certain embodiments, the conjugates of the present invention comprise two types of -L¹-. In certain embodiments, the conjugates of the present invention comprise three types of -L¹-. In certain embodiments, the conjugates of the present invention comprise four types of -L¹-.

In certain embodiments, the conjugates of the present invention comprise one type of -D and one type of -L¹-. In certain embodiments, the conjugates of the present invention comprise two types of -D and two types of -L¹-. In certain embodiments, the conjugates of the present invention comprise three types of -D and three types of -L¹-. In certain embodiments, the conjugates of the present invention comprise four types of -D and four types of -L¹-. In certain embodiments, the conjugates of the present invention comprise two types of -D and one type of -L¹-. In certain embodiments, the conjugates of the present invention comprise three types of -D and one type of -L¹-. In certain embodiments, the conjugates of the present invention comprise four types of -D and one type of -L¹-.

In certain embodiments, all moieties -L¹- of the conjugate have the same structure. In certain embodiments, the conjugate comprises two or more different types of moiety -L¹-, such as for example two, three, four or five different types of moiety -L¹-. Such two or more different types of moiety -L¹- may be conjugated to the same or different type of -D. Using different types of -L¹- allows releasing the same or different type of drug D-H from the conjugate of the present invention with different release half-lives, such as when combining a first group of moieties -L¹- with a short release half-live with a second group of moiety -L¹- with a long release half-life.

In certain embodiments, -D is selected from the group consisting of small molecule, medium size molecule, oligonucleotide, peptide nucleic acid, peptide and protein drug moieties.

In certain embodiments, -D is selected from the group consisting of small molecule, medium size, peptide and protein drug moieties.

In certain embodiments, -D is a small molecule drug moiety. In certain embodiments, such small molecule drug moiety is a nucleobase-based drug moiety.

In certain embodiments, -D is a medium size molecule drug moiety. In certain embodiments, -D is an oligonucleotide drug moiety. In certain embodiments, -D is a peptide nucleic acid protein drug moiety.

It is understood that a moiety -D comprises at least one primary or secondary amine group such as for example, one, two, three, four, five, six, seven, eight, nine or ten primary or secondary amine groups and that a moiety -D may also comprise one or more primary amine groups and one or more secondary amine groups.

In certain embodiments, -D is a peptide drug moiety.

In certain embodiments, -D is a peptide drug moiety selected from the group consisting of C-type natriuretic peptide, parathyroid hormone, W peptide, memno-peptide A and GI peptide.

In certain embodiments, -D is a bicyclic peptide drug moiety.

In certain embodiments, -D is a protein drug moiety. In certain embodiments, such protein moiety is a monoclonal or polyclonal antibody or fragment or fusion thereof.

It was surprisingly found that the conjugates of the present invention may be obtained via a method of synthesis that avoids the use of for example amide or amidine protecting groups while providing the benefit of obtaining stable reagents. In particular, this is beneficial for protein drug moieties, as some proteins are more prone to degradation under the deprotection conditions of amide or amidine protecting groups than for example small molecule, medium size and peptide drug moieties.

In certain embodiments, —X³— of formula (I) is —O—. In certain embodiments, —X³— of formula (I) is —S—. In certain embodiments, —X³— of formula (I) is —Se—.

In certain embodiments, —R⁶ of formula (I) is —H. In certain embodiments, —R⁶ of formula (I) is —C(R¹¹)(R^{11a})(R^{11b}). In certain embodiments, —R⁶ of formula (I) is -T.

In certain embodiments, —R^{6a} of formula (I) is —H. In certain embodiments, —R^{6a} of formula (I) is —C(R¹¹)(R^{11a})(R^{11b}). In certain embodiments, —R^{6a} of formula (I) is -T.

In certain embodiments, both —R⁶ and —R^{6a} of formula (I) are —H.

In certain embodiments, —X³— of formula (II) is —O—. In certain embodiments, —X³— of formula (II) is —S—. In certain embodiments, —X³— of formula (II) is —Se—.

In certain embodiments, —R⁶ of formula (II) is —PG and —R^{6a} of formula (II) is —H. In certain embodiments, —R⁶ of formula (II) is —PG and —R^{6a} of formula (II) is —C(R¹¹)(R^{11a})(R^{11b}) In certain embodiments, —R⁶ of formula (II) is —PG and —R^{6a} of formula (II) is -T. In certain embodiments, —R⁶ of formula (II) is —PG and —R^{6a} of formula (II) is —PG.

In certain embodiments, —R⁶ of formula (II) is —C(R¹¹)(R^{11a})(R^{11b}) and —R^{6a} of formula (II) is -T. In certain embodiments, —R⁶ and —R^{6a} of formula (II) are both —C(R¹¹)(R^{1a})(R^{11b}). In certain embodiments, —R⁶ and —R^{6a} of formula (II) are both -T.

In certain embodiments, —R^{A} of formula (II) is —H and —R^{B} of formula (II) is —PG.

In certain embodiments, —R⁶ of formula (II) forms with —R^{6a} of formula (II) a moiety —PG.

In certain embodiments, —R⁶ of formula (II) forms with —R^{A} of formula (II) a moiety —PG.

In certain embodiments, —R^{A} of formula (II) forms with —R^{B} of formula (II) a moiety —PG.

In certain embodiments, v of formula (I) or (II) is 0. In certain embodiments, v of formula (I) or (II) is 1.

In certain embodiments, —X¹— of formula (I) or (II) is —C(R⁸)(R^{8a})—. In certain embodiments, —X¹— of formula (I) or (II) is —N(R⁹)—. In certain embodiments, —X¹— of formula (I) or (II) is —O—.

In certain embodiments, ═X² of formula (I) or (II) is ═O. In certain embodiments, ═X² of formula (I) or (II) is ═N(R¹⁰).

In certain embodiments, —R⁹ of formula (I) or (II) is —C(R¹¹)(R^{11a})(R^{11b}). In certain embodiments, —R⁹ of formula (I) or (II) is -T.

In certain embodiments, —R¹⁰ of formula (I) or (II) is —H. In certain embodiments, —R¹⁰ of formula (I) or (II) is —C(R¹¹)(R^{11a})(R^{11b}). In certain embodiments, —R¹⁰ of formula (I) or (II) is -T.

In certain embodiments, —R¹ of formula (I) or (II) is selected from the group consisting of —H, halogen, —CN, —C(O)OR¹², —OR¹², —C(O)R¹², —C(O)N(R¹²)(R^{12a}), —S(O)₂N(R¹²)(R^{12a}), —S(O)N(R¹²)(R^{12a}), —S(O)₂R¹², —S(O)R¹², —N(R¹²)S(O)₂N(R^{12a})(R^{12b}), —SR¹², —NO₂, —N(R¹²)C(O)OR^{12a}, —N(R¹²)C(O)N(R^{12a})(R^{12b}), —OC(O)N(R¹²)(R^{12a}), -T, C₁₋₆ alkyl, C₂₋₆ alkenyl and C₂₋₆ alkynyl. In certain embodiments, —R¹ of formula (I) or (II) is —H. In certain embodiments, —R¹ of formula (I) or (II) is halogen. In certain embodiments, —R¹ of formula (I) or (II) is -T. In certain embodiments, —R¹ of formula (I) or (II) is C₁₋₆ alkyl. In certain embodiments, —R¹ of formula (I) or (II) is C₂₋₆ alkenyl. In certain embodiments, —R¹ of formula (I) or (II) is C₂₋₆ alkynyl. In certain embodiments, —R¹ of formula (I) or (II) is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl.

In certain embodiments, —R^{1a} of formula (I) or (II) is selected from the group consisting of —H, halogen, —CN, —C(O)OR¹², —OR¹², —C(O)R¹², —C(O)N(R¹²)(R^{12a}), —S(O)₂N(R¹²)(R^{12a}), —S(O)N(R¹²)(R^{12a}), —S(O)₂R¹², —S(O)R¹², —N(R¹²)S(O)₂N(R^{12a})(R^{12b}), —SR¹², —NO₂, —N(R¹²)C(O)OR^{12a}, —N(R¹²)C(O)N(R^{12a})(R^{12b}), —OC(O)N(R¹²)(R^{12a}), -T, C₁₋₆ alkyl, C₂₋₆ alkenyl and C₂₋₆ alkynyl. In certain embodiments, —R^{1a} of formula (I) or (II) is —H. In certain embodiments, —R^{1a} of formula (I) or (II) is halogen. In certain embodiments, —R^{1a} of formula (I) or (II) is -T. In certain embodiments, —R^{1a} of formula (I) or (II) is C₁₋₆ alkyl. In certain embodiments, —R^{1a} of formula (I) or (II) is C₂₋₆ alkenyl. In certain embodiments, —R^{1a} of formula (I) or (II) is C₂₋₆ alkynyl. In certain embodiments, —R^{1a} of formula (I) or (II) is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl.

In certain embodiments, —R² of formula (I) or (II) is selected from the group consisting of —H, halogen, —CN, —C(O)OR¹², —OR¹², —C(O)R¹², —C(O)N(R¹²)(R^{12a}), —S(O)₂N(R¹²)(R^{12a}), —S(O)N(R¹²)(R^{12a}), —S(O)₂R¹², —S(O)R¹², —N(R¹²)S(O)₂N(R^{12a})(R^{12b}), —SR¹², —NO₂, —N(R¹²)C(O)OR^{12a}, —N(R¹²)C(O)N(R^{12a})(R^{12b}), —OC(O)N(R¹²)(R^{12a}), -T, C₁₋₆ alkyl, C₂₋₆ alkenyl and C₂₋₆ alkynyl. In certain embodiments, —R² of formula (I) or (II) is —H. In certain embodiments, —R² of formula (I) or (II) is halogen. In certain embodiments, —R² of formula (I) or (II) is -T. In certain embodiments, —R² of formula (I) or (II) is C₁₋₆ alkyl. In certain embodiments, —R² of formula (I) or (II) is C₂₋₆ alkenyl. In certain embodiments, —R² of formula (I) or (II) is C₂₋₆ alkynyl. In certain embodiments, —R² of formula (I) or (II) is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl.

In certain embodiments, —R^{2a} of formula (I) or (II) is selected from the group consisting of —H, halogen, —CN, —C(O)OR¹², —OR¹², —C(O)R¹², —C(O)N(R¹²)(R^{12a}), —S(O)₂N(R¹²)(R^{12a}), —S(O)N(R¹²)(R^{12a}), —S(O)₂R¹², —S(O)R¹², —N(R¹²)S(O)₂N(R^{12a})(R^{12b}), —SR¹², —NO₂, —N(R¹²)C(O)OR^{12a}, —N(R¹²)C(O)N(R^{12a})(R^{12b}), —OC(O)N(R¹²)(R^{12a}), -T, C₁₋₆ alkyl, C₂₋₆ alkenyl and C₂₋₆ alkynyl. In certain embodiments, —R^{2a} of formula (I) or (II) is —H. In certain embodiments, —R^{2a} of formula (I) or (II) is halogen. In certain embodiments, —R^{2a} of formula (I) or (II) is -T. In certain embodiments, —R^{2a} of formula (I) or (II) is C₁₋₆ alkyl. In certain embodiments, —R^{2a} of formula (I) or (II) is C₂₋₆ alkenyl. In certain embodiments, —R^{2a} of formula (I) or (II) is C₂₋₆ alkynyl. In certain embodiments, —R^{2a} of formula (I) or (II) is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl.

In certain embodiments, —R³ of formula (I) or (II) is selected from the group consisting of —H, halogen, —CN, —C(O)OR¹², —OR¹², —C(O)R¹², —C(O)N(R¹²)(R^{12a}), —S(O)₂N(R¹²)(R^{12a}), —S(O)N(R¹²)(R^{12a}), —S(O)₂R¹², —S(O)R¹², —N(R¹²)S(O)₂N(R^{12a})(R^{12b}), —SR¹², —NO₂, —N(R¹²)C(O)OR^{12a}, —N(R¹²)C(O)N(R^{12a})(R^{12b}), —OC(O)N(R¹²)(R^{12a}), -T, C₁₋₆ alkyl, C₂₋₆ alkenyl and C₂₋₆ alkynyl. In certain embodiments, —R³ of formula (I) or (II) is —H. In certain embodiments, —R³ of formula (I) or (II) is halogen. In certain embodiments, —R³ of formula (I) or (II) is -T. In certain embodiments, —R³ of formula (I) or (II) is C₁₋₆ alkyl. In certain embodiments, —R³ of formula (I) or (II) is C₂₋₆ alkenyl. In certain embodiments, —R³ of formula (I) or (II) is C₂₋₆ alkynyl. In certain embodiments, —R³ of formula (I) or (II) is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl.

In certain embodiments, —R^{3a} of formula (I) or (II) is selected from the group consisting of —H, halogen, —CN, —C(O)OR¹², —OR¹², —C(O)R¹², —C(O)N(R¹²)(R^{12a}), —S(O)₂N(R¹²)(R^{12a}), —S(O)N(R¹²)(R^{12a}), —S(O)₂R¹², —S(O)R¹², —N(R¹²)S(O)₂N(R^{12a})(R^{12b}), —SR¹², —NO₂, —N(R¹²)C(O)OR^{12a}, —N(R¹²)C(O)N(R^{12a})(R^{12b}), —OC(O)N(R¹²)(R^{12a}), -T, C₁₋₆ alkyl, C₂₋₆ alkenyl and C₂₋₆ alkynyl. In certain embodiments, —R^{3a} of formula (I) or (II) is —H. In certain embodiments, —R^{3a} of formula (I) or (II) is halogen. In certain embodiments, —R^{3a} of formula (I) or (II) is -T. In certain embodiments, —R^{3a} of formula (I) or (II) is C₁₋₆ alkyl. In certain embodiments, —R^{3a} of formula (I) or (II) is C₂₋₆ alkenyl. In certain embodiments, —R^{3a} of formula (I) or (II) is C₂₋₆ alkynyl. In certain embodiments, —R^{3a} of formula (I) or (II) is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl.

In certain embodiments, —R⁴ of formula (I) or (II) is selected from the group consisting of —H, halogen, —CN, —C(O)OR¹², —OR¹², —C(O)R¹², —C(O)N(R¹²)(R^{12a}), —S(O)₂N(R¹²)(R^{12a}), —S(O)N(R¹²)(R^{12a}), —S(O)₂R¹², —S(O)R¹², —N(R¹²)S(O)₂N(R^{12a})(R^{12b}), —SR¹², —NO₂, —N(R¹²)C(O)OR^{12a}, —N(R¹²)C(O)N(R^{12a})(R^{12b}), —OC(O)N(R¹²)(R^{12a}), -T, C₁₋₆ alkyl, C₂₋₆ alkenyl and C₂₋₆ alkynyl. In certain embodiments, —R⁴ of formula (I) or (II) is —H. In certain embodiments, —R⁴ of formula (I) is halogen. In certain embodiments, —R⁴ of formula (I) or (II) is -T. In certain embodiments, —R⁴ of formula (I) or (II) is C₁₋₆ alkyl. In certain embodiments, —R⁴ of formula (I) or (II) is C₂₋₆ alkenyl. In certain embodiments, —R⁴ of formula (I) or (II) is C₂₋₆ alkynyl. In certain embodiments, —R⁴ of formula (I) or (II) is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl.

In certain embodiments, —R^{4a} of formula (I) or (II) is selected from the group consisting of —H, halogen, —CN, —C(O)OR¹², —OR¹², —C(O)R¹², —C(O)N(R¹²)(R^{12a}), —S(O)₂N(R¹²)(R^{12a}), —S(O)N(R¹²)(R^{12a}), —S(O)₂R¹², —S(O)R¹², —N(R¹²)S(O)₂N(R^{12a})(R^{12b}), —SR¹², —NO₂, —N(R¹²)C(O)OR^{12a}, —N(R¹²)C(O)N(R^{12a})(R^{12b}), —OC(O)N(R¹²)(R^{12a}), -T, C₁₋₆ alkyl, C₂₋₆ alkenyl and C₂₋₆ alkynyl. In certain embodiments, —R^{4a} of formula (I) or (II) is —H. In certain embodiments, —R^{4a} of formula (I) or (II) is halogen. In certain embodiments, —R^{4a} of formula (I) or (II) is -T. In certain embodiments, —R^{4a} of formula (I) or (II) is C₁₋₆ alkyl. In certain embodiments, —R^{4a} of formula (I) or (II) is C₂₋₆ alkenyl. In certain embodiments, —R^{4a} of formula (I) or (II) is C₂₋₆ alkynyl. In certain embodiments, —R^{4a} of formula (I) or (II) is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl.

In certain embodiments, —R⁵ of formula (I) or (II) is selected from the group consisting of —H, halogen, —CN, —C(O)OR¹², —OR¹², —C(O)R¹², —C(O)N(R¹²)(R^{12a}), —S(O)₂N(R¹²)(R^{12a}), —S(O)N(R¹²)(R^{12a}), —S(O)₂R¹², —S(O)R¹², —N(R¹²)S(O)₂N(R^{12a})(R^{12b}), —SR¹², —NO₂, —N(R¹²)C(O)OR^{12a}, —N(R¹²)C(O)N(R^{12a})(R^{12b}), —OC(O)N(R¹²)(R^{12a}), -T, C₁₋₆ alkyl, C₂₋₆ alkenyl and C₂₋₆ alkynyl. In certain embodiments, —R⁵ of formula (I) or (II) is —H. In certain embodiments, —R⁵ of formula (I) or (II) is halogen. In certain embodiments, —R⁵ of formula (I) or (II) is -T. In certain embodiments, —R⁵ of formula (I) or (II) is C₁₋₆ alkyl. In certain embodiments, —R⁵ of formula (I) or (II) is C₂₋₆ alkenyl. In certain embodiments, —R⁵ of formula (I) or (II) is C₂₋₆ alkynyl. In certain embodiments, —R⁵ of formula (I) or (II) is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl.

In certain embodiments, —R^{5a} of formula (I) or (II) is selected from the group consisting of —H, halogen, —CN, —C(O)OR¹², —OR¹², —C(O)R¹², —C(O)N(R¹²)(R^{12a}), —S(O)₂N(R¹²)(R^{12a}), —S(O)N(R¹²)(R^{12a}), —S(O)₂R¹², —S(O)R¹², —N(R¹²)S(O)₂N(R^{12a})(R^{12b}), —SR¹², —NO₂, —N(R¹²)C(O)OR^{12a}, —N(R¹²)C(O)N(R^{12a})(R^{12b}), —OC(O)N(R¹²)(R^{12a}), -T, C₁₋₆ alkyl, C₂₋₆ alkenyl and C₂₋₆ alkynyl. In certain embodiments, —R^{5a} of formula (I) or (II) is —H. In certain embodiments, —R^{8a} of formula (I) or (II) is halogen. In certain embodiments, —R^{8a} of formula (I) or (II) is -T. In certain embodiments, —R^{8a} of formula (I) or (II) is C₁₋₆ alkyl. In certain embodiments, —R^{8a} of formula (I) or (II) is C₂₋₆ alkenyl. In certain embodiments, —R^{8a} of formula (I) or (II) is C₂₋₆ alkynyl. In certain embodiments, —R^{8a} of formula (I) or (II) is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl.

In certain embodiments, —R⁷ of formula (I) or (II) is selected from the group consisting of —H, halogen, —CN, —C(O)OR¹², —OR¹², —C(O)R¹², —C(O)N(R¹²)(R^{12a}), —S(O)₂N(R¹²)(R^{12a}), —S(O)N(R¹²)(R^{12a}), —S(O)₂R¹², —S(O)R¹², —N(R¹²)S(O)₂N(R^{12a})(R^{12b}), —SR¹², —NO₂, —N(R¹²)C(O)OR^{12a}, —N(R¹²)C(O)N(R^{12a})(R^{12b}), —OC(O)N(R¹²)(R^{12a}), -T, C₁₋₆ alkyl, C₂₋₆ alkenyl and C₂₋₆ alkynyl. In certain embodiments, —R⁷ of formula (I) or (II) is —H. In certain embodiments, —R⁷ of formula (I) or (II) is halogen. In certain embodiments, —R⁷ of formula (I) or (II) is -T. In certain embodiments, —R⁷ of formula (I) or (II) is C₁₋₆ alkyl. In certain embodiments, —R⁷ of formula (I) or (II) is C₂₋₆ alkenyl. In certain embodiments, —R⁷ of formula (I) or (II) is C₂₋₆ alkynyl. In certain embodiments, —R⁷ of formula (I) or (II) is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl.

In certain embodiments, —R⁸ of formula (I) or (II) is selected from the group consisting of —H, halogen, —CN, —C(O)OR¹², —OR¹², —C(O)R¹², —C(O)N(R¹²)(R^{12a}), —S(O)₂N(R¹²)(R^{12a}), —S(O)N(R¹²)(R^{12a}), —S(O)₂R¹², —S(O)R¹², —N(R¹²)S(O)₂N(R^{12a})(R^{12b}), —SR¹², —NO₂, —N(R¹²)C(O)OR^{12a}, —N(R¹²)C(O)N(R^{12a})(R^{12b}), —OC(O)N(R¹²)(R^{12a}), -T, C₁₋₆ alkyl, C₂₋₆ alkenyl and C₂₋₆ alkynyl. In certain embodiments, —R⁸ of formula (I) or (II) is —H. In certain embodiments, —R⁸ of formula (I) or (II) is halogen. In certain embodiments, —R⁸ of formula (I) or (II) is -T. In certain embodiments, —R⁸ of formula (I) or (II) is C₁₋₆ alkyl. In certain embodiments, —R⁸ of formula (I) or (II) is C₂₋₆ alkenyl. In certain embodiments, —R⁸ of formula (I) or (II) is C₂₋₆ alkynyl. In certain embodiments, —R⁸ of formula (I) or (II) is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl.

In certain embodiments, —R^{8a} of formula (I) or (II) is selected from the group consisting of —H, halogen, —CN, —C(O)OR¹², —OR¹², —C(O)R¹², —C(O)N(R¹²)(R^{12a}), —S(O)₂N(R¹²)(R^{12a}), —S(O)N(R¹²)(R^{12a}), —S(O)₂R¹², —S(O)R¹², —N(R¹²)S(O)₂N(R^{12a})(R^{12b}), —SR¹², —NO₂, —N(R¹²)C(O)OR^{12a}, —N(R¹²)C(O)N(R^{12a})(R^{12b}), —OC(O)N(R¹²)(R^{12a}), -T, C₁₋₆ alkyl, C₂₋₆ alkenyl and C₂₋₆ alkynyl. In certain embodiments, —R^{8a} of formula (I) or (II) is —H. In certain embodiments, —R^{8a} of formula (I) or (II) is halogen. In certain embodiments, —R^{8a} of formula (I) or (II) is -T. In certain embodiments, —R^{8a} of formula (I) or (II) is C₁₋₆ alkyl. In certain embodiments, —R^{8a} of formula (I) or (II) is C₂₋₆ alkenyl. In certain embodiments, —R^{8a} of formula (I) or (II) is C₂₋₆ alkynyl. In certain embodiments, —R^{8a} of formula (I) or (II) is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl.

In certain embodiments, —R¹¹ of formula (I) or (II) is selected from the group consisting of —H, halogen, —CN, —C(O)OR¹², —OR¹², —C(O)R¹², —C(O)N(R¹²)(R^{12a}), —S(O)₂N(R¹²)(R^{12a}), —S(O)N(R¹²)(R^{12a}), —S(O)₂R¹², —S(O)R¹², —N(R¹²)S(O)₂N(R^{12a})(R^{12b}), —SR¹², —NO₂, —N(R¹²)C(O)OR^{12a}, —N(R¹²)C(O)N(R^{12a})(R^{12b}), —OC(O)N(R¹²)(R^{12a}), -T, C₁₋₆ alkyl, C₂₋₆ alkenyl and C₂₋₆ alkynyl. In certain embodiments, —R¹¹ of formula (I) or (II) is —H. In certain embodiments, —R¹¹ of formula (I) or (II) is halogen. In certain embodiments, —R¹¹ of formula (I) or (II) is -T. In certain embodiments, —R¹¹ of formula (I) or (II) is C₁₋₆ alkyl. In certain embodiments, —R¹¹ of formula (I) or (II) is C₂₋₆ alkenyl. In certain embodiments, —R¹¹ of formula (I) or (II) is C₂₋₆ alkynyl. In certain embodiments, —R¹¹ of formula (I) or (II) is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl.

In certain embodiments, —R^{11a} of formula (I) or (II) is selected from the group consisting of —H, halogen, —CN, —C(O)OR¹², —OR¹², —C(O)R¹², —C(O)N(R¹²)(R^{12a}), —S(O)₂N(R¹²)(R^{12a}), —S(O)N(R¹²)(R^{12a}), —S(O)₂R¹², —S(O)R¹², —N(R¹²)S(O)₂N(R^{12a})(R^{12b}), —SR¹², —NO₂, —N(R¹²)C(O)OR^{12a}, —N(R¹²)C(O)N(R^{12a})(R^{12b}), —OC(O)N(R¹²)(R^{12a}), -T, C₁₋₆ alkyl, C₂₋₆ alkenyl and C₂₋₆ alkynyl. In certain embodiments, —R^{11a} of formula (I) or (II) is —H. In certain embodiments, —R^{11a} of formula (I) or (II) is halogen. In certain embodiments, —R^{11a} of formula (I) or (II) is -T. In certain embodiments, —R^{11a} of formula (I) or (II) is C₁₋₆ alkyl. In certain embodiments, —R^{11a} of formula (I) or (II) is C₂₋₆ alkenyl. In certain embodiments, —R^{11a} of formula (I) or (II) is C₂₋₆ alkynyl. In certain embodiments, —R^{11a} of formula (I) or (II) is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl.

In certain embodiments, —R^{11b} of formula (I) or (II) is selected from the group consisting of —H, halogen, —CN, —C(O)OR¹², —OR¹², —C(O)R¹², —C(O)N(R¹²)(R^{12a}), —S(O)₂N(R¹²)(R^{12a}), —S(O)N(R¹²)(R^{12a}), —S(O)₂R¹², —S(O)R¹², —N(R¹²)S(O)₂N(R^{12a})(R^{12b}), —SR¹², —NO₂, —N(R¹²)C(O)OR^{12a}, —N(R¹²)C(O)N(R^{12a})(R^{12b}), —OC(O)N(R¹²)(R^{12a}), -T, C₁₋₆ alkyl, C₂₋₆ alkenyl and C₂₋₆ alkynyl. In certain embodiments, —R^{11b} of formula (I) or (II) is —H. In certain embodiments, —R^{11b} of formula (I) or (II) is halogen. In certain embodiments, —R^{11b} of formula (I) or (II) is -T. In certain embodiments, —R^{11b} of formula (I) or (II) is C₁₋₆ alkyl. In certain embodiments, —R^{11b} of formula (I) or (II) is C₂₋₆ alkenyl. In certain embodiments, —R^{11b} of formula (I) or (II) is C₂₋₆ alkynyl. In certain embodiments, —R^{11b} of formula (I) or (II) is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl.

In certain embodiments, —R¹² of formula (I) or (II) is selected from the group consisting of —H, -T, C₁₋₆ alkyl, C₂₋₆ alkenyl and C₂₋₆ alkynyl. In certain embodiments, —R¹² of formula (I) or (II) is —H. In certain embodiments, —R¹² of formula (I) or (II) is -T. In certain embodiments, —R¹² of formula (I) or (II) is C₁₋₆ alkyl. In certain embodiments, —R¹² of formula (I) or (II) is C₂₋₆ alkenyl. In certain embodiments, —R¹² of formula (I) or (II) is C₂₋₆ alkynyl.

In certain embodiments, —R^{12a} of formula (I) or (II) is selected from the group consisting of —H, -T, C₁₋₆ alkyl, C₂₋₆ alkenyl and C₂₋₆ alkynyl. In certain embodiments, —R^{12a} of formula (I) or (II) is —H. In certain embodiments, —R^{12a} of formula (I) or (II) is -T. In certain embodiments, —R^{12a} of formula (I) or (II) is C₁₋₆ alkyl. In certain embodiments, —R^{12a} of formula (I) or (II) is C₂₋₆ alkenyl. In certain embodiments, —R^{12a} of formula (I) or (II) is C₂₋₆ alkynyl.

In certain embodiments, —R^{12b} of formula (I) or (II) is selected from the group consisting of —H, -T, C₁₋₆ alkyl, C₂₋₆ alkenyl and C₂₋₆ alkynyl. In certain embodiments, —R^{12b} of formula (I) or (II) is —H. In certain embodiments, —R^{12b} of formula (I) or (II) is -T. In certain embodiments, —R^{12b} of formula (I) or (II) is C₁₋₆ alkyl. In certain embodiments, —R^{12b} of formula (I) or (II) is C₂₋₆ alkenyl. In certain embodiments, —R^{12b} of formula (I) or (II) is C₂₋₆ alkynyl.

In certain embodiments, T of formula (I) or (II) is selected from the group consisting of phenyl, naphthyl, indenyl, indanyl, tetralinyl, C₃₋₁₀ cycloalkyl, 3- to 10-membered heterocyclyl and 8- to 11-membered heterobicyclyl. In certain embodiments, T of formula (I) or (II) is phenyl.

In certain embodiments, T of formula (I) or (II) is naphthyl. In certain embodiments, T of formula (I) or (II) is indenyl. In certain embodiments, T of formula (I) or (II) is indanyl. In certain embodiments, T of formula (I) or (II) is tetralinyl. In certain embodiments, T of formula (I) or (II) is tetralinyl. In certain embodiments, T of formula (I) or (II) is C₃₋₁₀ cycloalkyl. In certain embodiments, T of formula (I) or (II) is 3- to 10-membered heterocyclyl. In certain embodiments, T of formula (I) or (II) is 8- to 11-membered heterobicyclyl.

In certain embodiments, T of formula (I) or (II) is substituted with one or more —R¹³ of formula (I) or (II), which are the same of different.

In certain embodiments, T of formula (I) or (II) is substituted with one —R¹³ of formula (I) or (II).

In certain embodiments, T of formula (I) or (II) is not substituted with —R¹³.

In certain embodiments, —R¹³ of formula (I) or (II) is selected from the group consisting of halogen, —CN, oxo, —C(O)OR¹⁵, —OR¹⁵, —C(O)R¹⁵, —C(O)N(R¹⁵)(R^{15a}), —S(O)₂N(R¹⁵)(R^{15a}), —S(O)N(R¹⁵)(R^{15a}), —S(O)₂R¹⁵, —S(O)R¹⁵, —N(R¹⁵)S(O)₂N(R^{15a})(R^{15b}), —SR¹⁵, —N(R¹⁵)(R^{15a}), —NO₂, —OC(O)R¹⁵, —N(R¹⁵)C(O)R^{15a}, —N(R¹⁵)S(O)₂R^{1a}, —N(R¹⁵)S(O)R^{15a}, —N(R¹⁵)C(O)OR^{15a}, —N(R¹⁵)C(O)N(R^{15a})(R^{15b}), —OC(O)N(R¹⁵)(R^{15a}) and C₁₋₆ alkyl. In certain embodiments, —R¹³ of formula (I) or (II) is halogen. In certain embodiments, —R¹³ of formula (I) or (II) is —CN. In certain embodiments, —R¹³ of formula (I) or (II) is oxo. In certain embodiments, —R¹³ of formula (I) or (II) is —C(O)OR¹⁵. In certain embodiments, —R¹³ of formula (I) or (II) is —OR¹⁵. In certain embodiments, —R¹³ of formula (I) or (II) is —C(O)R¹⁵. In certain embodiments, —R¹³ of formula (I) or (II) is —C(O)N(R¹⁵)(R^{15a}) In certain embodiments, —R¹³ of formula (I) or (II) is —S(O)₂N(R¹⁵)(R^{15a}). In certain embodiments, —R¹³ of formula (I) or (II) is —S(O)N(R¹⁵)(R^{15a}). In certain embodiments, —R¹³ of formula (I) or (II) is —S(O)₂R¹⁵. In certain embodiments, —R¹³ of formula (I) or (II) is —S(O)R¹⁵. In certain embodiments, —R¹³ of formula (I) or (II) is —N(R¹⁵)S(O)₂N(R^{15a})(R^{15b}). In certain embodiments, —R¹³ of formula (I) or (II) is —SR¹⁵. In certain embodiments, —R¹³ of formula (I) or (II) is —N(R¹⁵)(R^{15a}). In certain embodiments, —R¹³ of formula (I) or (II) is —NO₂. In certain embodiments, —R¹³ of formula (I) or (II) is —OC(O)R¹⁵.

In certain embodiments, —R¹³ of formula (I) or (II) is —N(R¹⁵)C(O)R^{15a}. In certain embodiments, —R¹³ of formula (I) or (II) is —N(R¹⁵)S(O)₂R^{15a}. In certain embodiments, —R¹³ of formula (I) or (II) is —N(R¹⁵)S(O)R^{15a}. In certain embodiments, —R¹³ of formula (I) or (II) is —N(R¹⁵)C(O)OR^{15a}. In certain embodiments, —R¹³ of formula (I) or (II) is —N(R¹⁵)C(O)N(R^{15a})(R^{15b}). In certain embodiments, —R¹³ of formula (I) or (II) is —OC(O)N(R¹⁵)(R^{15a}). In certain embodiments, —R¹³ of formula (I) or (II) is C₁₋₆ alkyl.

In certain embodiments, —R¹⁴ of formula (I) or (II) is selected from the group consisting of —H and C₁₋₆ alkyl. In certain embodiments, —R¹⁴ of formula (I) or (II) is —H. In certain embodiments, —R¹⁴ of formula (I) or (II) is C₁₋₆ alkyl.

In certain embodiments, —R^{14a} of formula (I) or (II) is selected from the group consisting of —H and C₁₋₆ alkyl. In certain embodiments, —R^{14a} of formula (I) or (II) is —H. In certain embodiments, —R^{14a} of formula (I) or (II) is C₁₋₆ alkyl.

In certain embodiments, —R¹⁵ of formula (I) or (II) is selected from the group consisting of —H and C₁₋₆ alkyl. In certain embodiments, —R¹⁵ of formula (I) or (II) is —H. In certain embodiments, —R¹⁵ of formula (I) or (II) is C₁₋₆ alkyl.

In certain embodiments, —R^{15a} of formula (I) or (II) is selected from the group consisting of —H and C₁₋₆ alkyl. In certain embodiments, —R^{15a} of formula (I) or (II) is —H. In certain embodiments, —R^{15a} of formula (I) or (II) is C₁₋₆ alkyl.

In certain embodiments, —R^{15b} of formula (I) or (II) is selected from the group consisting of —H and C₁₋₆ alkyl. In certain embodiments, —R^{15b} of formula (I) or (II) is —H. In certain embodiments, —R^{15b} of formula (I) or (II) is C₁₋₆ alkyl.

In certain embodiments, —R¹ and —R^{1a} of formula (I) or (II) are joined together with the atom to which they are attached to form C₃₋₁₀ cycloalkyl. In certain embodiments, —R¹ and —R^{1a} of formula (I) or (II) are joined together with the atom to which they are attached to form a 3- to 10-membered heterocyclyl. In certain embodiments, —R¹ and —R^{1a} of formula (I) or (II) are joined together with the atom to which they are attached to form an 8- to 11-membered heterobicyclyl.

In certain embodiments, —R² and —R^{2a} of formula (I) or (II) are joined together with the atom to which they are attached to form C₃₋₁₀ cycloalkyl. In certain embodiments, —R² and —R^{2a} of formula (I) or (II) are joined together with the atom to which they are attached to form a 3- to 10-membered heterocyclyl. In certain embodiments, —R² and —R^{2a} of formula (I) or (II) are joined together with the atom to which they are attached to form an 8- to 11-membered heterobicyclyl.

In certain embodiments, —R³ and —R^{3a} of formula (I) or (II) are joined together with the atom to which they are attached to form C₃₋₁₀ cycloalkyl. In certain embodiments, —R³ and —R^{3a} of formula (I) or (II) are joined together with the atom to which they are attached to form a 3- to 10-membered heterocyclyl. In certain embodiments, —R³ and —R^{3a} of formula (I) or (II) are joined together with the atom to which they are attached to form an 8- to 11-membered heterobicyclyl.

In certain embodiments, —R⁴ and —R^{4a} of formula (I) or (II) are joined together with the atom to which they are attached to form C₃₋₁₀ cycloalkyl. In certain embodiments, —R⁴ and —R^{4a} of formula (I) or (II) are joined together with the atom to which they are attached to form a 3- to 10-membered heterocyclyl. In certain embodiments, —R⁴ and —R^{4a} of formula (I) or (II) are joined together with the atom to which they are attached to form an 8- to 11-membered heterobicyclyl.

In certain embodiments, —R⁵ and —R^{5a} of formula (I) or (II) are joined together with the atom to which they are attached to form C₃₋₁₀ cycloalkyl. In certain embodiments, —R⁵ and —R^{5a} of formula (I) or (II) are joined together with the atom to which they are attached to form a 3- to 10-membered heterocyclyl. In certain embodiments, —R⁵ and —R^{5a} of formula (I) or (II) are joined together with the atom to which they are attached to form an 8- to 11-membered heterobicyclyl.

In certain embodiments, —R⁸ and —R^{8a} of formula (I) or (II) are joined together with the atom to which they are attached to form C₃₋₁₀ cycloalkyl. In certain embodiments, —R⁸ and —R^{8a} of formula (I) or (II) are joined together with the atom to which they are attached to form a 3- to 10-membered heterocyclyl. In certain embodiments, —R⁸ and —R^{8a} of formula (I) or (II) are joined together with the atom to which they are attached to form an 8- to 11-membered heterobicyclyl.

In certain embodiments, —R¹ and —R² of formula (I) or (II) are joined together with the atoms to which they are attached to form a ring -A- of formula (I) or (II).

In certain embodiments, —R¹ and —R⁸ of formula (I) or (II) are joined together with the atoms to which they are attached to form a ring -A- of formula (I) or (II).

In certain embodiments, —R¹ and —R⁹ of formula (I) or (II) are joined together with the atoms to which they are attached to form a ring -A- of formula (I) or (II).

In certain embodiments, —R² and —R⁹ of formula (I) or (II) are joined together with the atoms to which they are attached to form a ring -A- of formula (I) or (II).

In certain embodiments, —R² and —R¹⁰ of formula (I) or (II) are joined together with the atoms to which they are attached to form a ring -A- of formula (I) or (II).

In certain embodiments, -A- of formula (I) or (II) is phenyl. In certain embodiments, -A- of formula (I) or (II) is naphthyl. In certain embodiments, -A- of formula (I) or (II) is indenyl. In certain embodiments, -A- of formula (I) or (II) is indanyl. In certain embodiments, -A- of formula (I) or (II) is tetralinyl. In certain embodiments, -A- of formula (I) or (II) is C₃₋₁₀ cycloalkyl. In certain embodiments, -A- of formula (I) or (II) is 3- to 10-membered heterocyclyl. In certain embodiments, -A- of formula (I) or (II) is 8- to 11-membered heterobicyclyl.

In certain embodiments, —R¹ and —R⁹ of formula (I) or (II) are joined together with the atoms to which they are attached to form a ring -A′- of formula (I) or (II).

In certain embodiments, —R² and —R⁹ of formula (I) or (II) are joined together with the atoms to which they are attached to form a ring -A′- of formula (I) or (II).

In certain embodiments, —R³ and —R⁶ of formula (I) or (II) are joined together with the atoms to which they are attached to form a ring -A′- of formula (I) or (II).

In certain embodiments, —R⁴ and —R⁶ of formula (I) or (II) are joined together with the atoms to which they are attached to form a ring -A′- of formula (I) or (II).

In certain embodiments, —R⁵ and —R⁶ of formula (I) or (II) are joined together with the atoms to which they are attached to form a ring -A′- of formula (I) or (II).

In certain embodiments, —R⁶ and —R^{6a} of formula (I) or (II) are joined together with the atoms to which they are attached to form a ring -A′- of formula (I) or (II).

In certain embodiments, —R⁶ and —R⁷ of formula (I) or (II) are joined together with the atoms to which they are attached to form a ring -A′- of formula (I) or (II).

In certain embodiments, -A′- of formula (I) or (II) is a 3- to 10-membered heterocyclyl. In certain embodiments, -A′- of formula (I) or (II) is an 8- to 11-membered heterobicyclyl.

In certain embodiments, —PG is selected from the group consisting of:

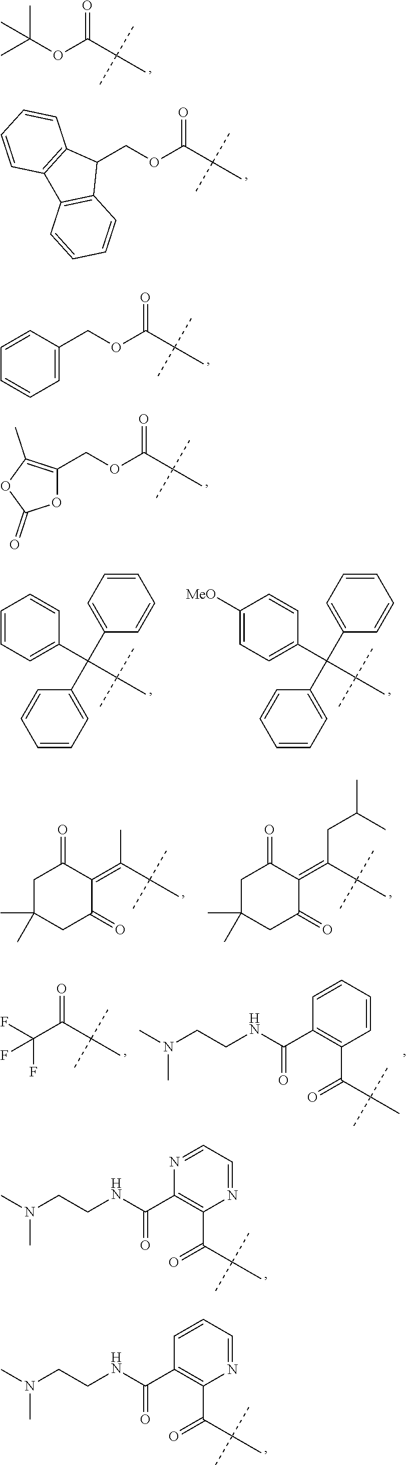

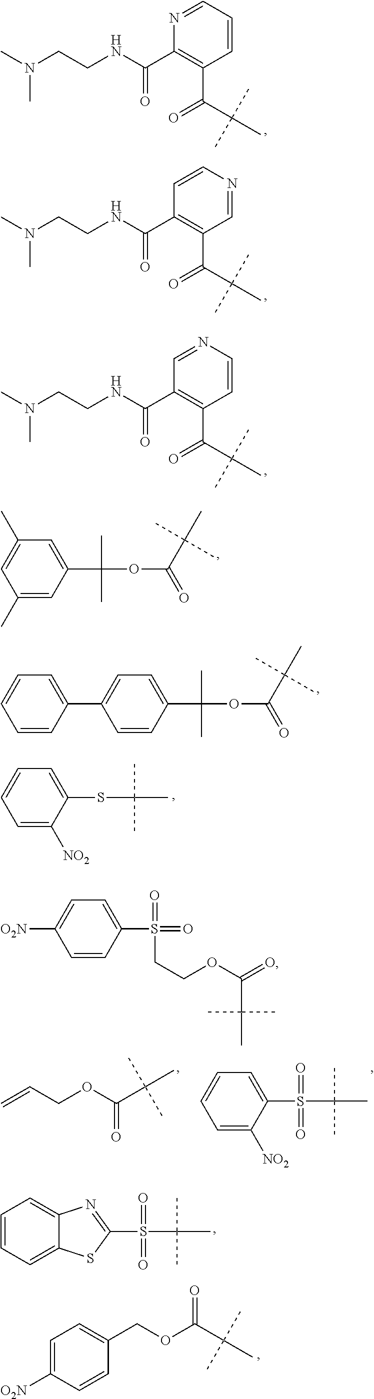

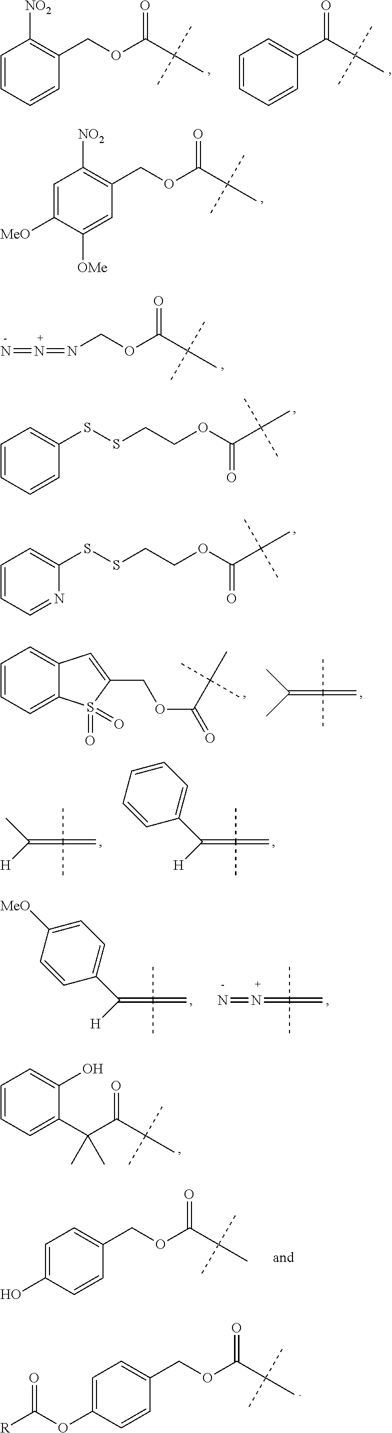

wherein
the dashed line indicates the attachment to a nitrogen atom of formula (II) that may be substituted with —PG and —R is C₁₋₆ alkyl.

In certain embodiments, —PG is a reversible prodrug linker moiety as disclosed in WO 2005/099768 A2. Accordingly, —PG is of formula (b-i):

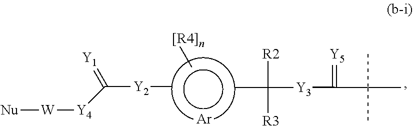

wherein
the dashed line indicates attachment to a nitrogen atom of formula (II) that may be substituted with —PG;
n is 0, 1, 2, 3, or 4;
—X— is a chemical bond or a spacer;
═Y₁, ═Y₅ are independently selected from the group consisting of ═O and ═S;
—Y₂—, —Y₃— are independently selected from the group consisting of —O— and —S—;
—Y₄— is selected from the group consisting of —O—, —NR⁵— and —C(R⁶R^{6a})—;
—R², —R³, —R⁵, —R⁶, —R^{6a} are independently of each other selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl;
—R⁴ is selected from the group consisting of methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl; —W— is selected from the group consisting of C₁₋₂₀ alkyl optionally interrupted by one or more groups selected from the group consisting of C₃₋₁₀ cycloalkyl, 8- to 30-membered carbopolycyclyl, 3- to 10-membered heterocyclyl, —C(O)—, —C(O)N(R⁷)—, —O—, —S— and —N(R⁷)—;
—Nu is a nucleophile;
—Ar— is a multi-substituted aromatic hydrocarbon or a multi-substituted aromatic heterocycle.

It is understood that the dashed line in formula (b-i) indicates attachment to one of the nitrogen atoms of formula (II) to which a moiety —PG may be attached to.

Optionally, —PG of formula (b-i) is further substituted.

In certain embodiments, ═Y₅ of formula (b-i) is ═O.

In certain embodiments, —Y₃— of formula (b-i) is —O—.

In certain embodiments, —R², —R³ and —R⁴ of formula (b-i) are independently selected from —H, C₁₋₆ alkyl, C₂₋₆ alkenyl or C₂₋₆ alkynyl, which C₁₋₆ alkyl, C₂₋₆ alkenyl or C₂₋₆ alkynyl are optionally further substituted.

In certain embodiments, ═Y₁ of formula (b-i) is ═O.

In certain embodiments, —Y₂— of formula (b-i) is —O—.

In certain embodiments, —Y₄— of formula (b-i) is —NR⁵.

In certain embodiments, —R⁵ of formula (b-i) is —H or C₁₋₆ alkyl.

In certain embodiments, Ar of formula (b-i) is selected from the group consisting of:

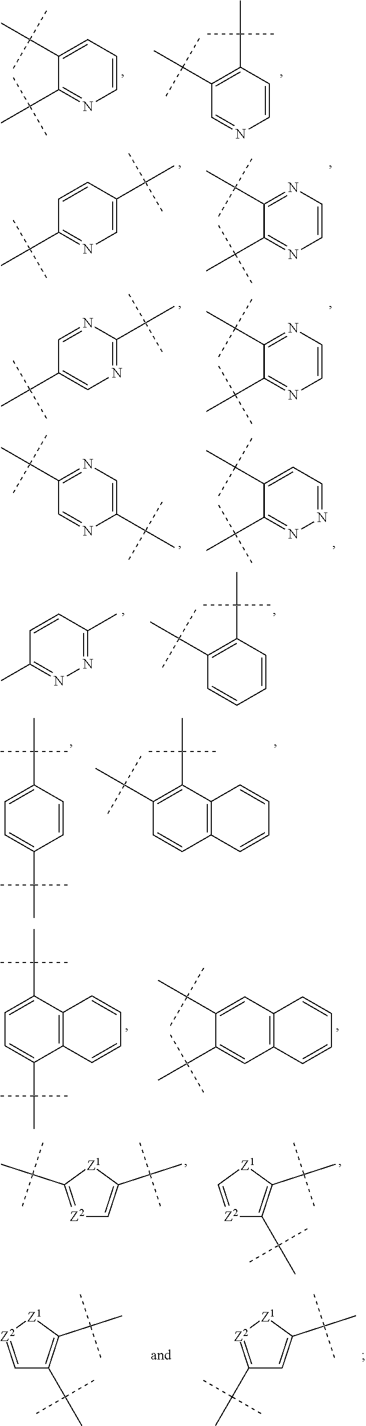

wherein
—Z¹— is selected from the group consisting of —O—, —S— and —N(R⁷)—, and
—Z²— is —N(R⁷)—; and
—R⁷ is selected from the group consisting of —H, C₁₋₆ alkyl, C₂₋₆ alkenyl and C₂₋₆ alkynyl.

In certain embodiments, the moiety

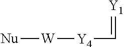

of formula (b-i) is selected from the group consisting of

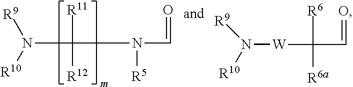

wherein
—W—, —R⁵, —R⁶, —R^{6a} are used as defined above;
m is 2, 3, 4, 5, 6, 7, 8, 9 or 10;
—R⁹, —R¹⁰, —R¹¹ and —R¹² are independently selected from the group consisting of H, C₁₋₆ alkyl, C₂₋₆ alkenyl and C₂₋₆ alkynyl, and which C₁₋₆ alkyl, C₂₋₆ alkenyl and C₂₋₆ alkynyl are optionally further substituted.

In certain embodiments, —PG is of formula (b-i′):

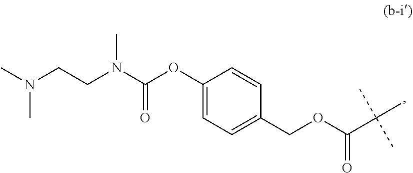

wherein
the dashed line indicates attachment to a nitrogen atom of formula (II) that may be substituted by —PG.

In certain embodiments, -Q is —OH.

In certain embodiments, -Q is -LG.

In certain embodiments, -LG is selected from the group consisting of chloride, bromide, fluoride, nitrophenoxy, imidazolyl, N-hydroxysuccinimidyl, N-hydroxybenzotriazolyl, N-hydroxyazobenzotriazolyl, pentafluorophenoxy, N-hydroxysulfosuccinimidyl, diphenylphosphinomethanethiyl, 2-diphenylphosphinophenoxy, norbornene-N-hydroxysuccinimidyl, N-hydroxyphthalimide, pyridinoxy, nonafluoro tert-butyloxy and hexafluoro isopropyloxy.

In certain embodiments, -LG is chloride. In certain embodiments, -LG is bromide. In certain embodiments, -LG is fluoride. In certain embodiments, -LG is nitrophenoxy. In certain embodiments, -LG is imidazolyl. In certain embodiments, -LG is N-hydroxysuccinimidyl. In certain embodiments, -LG is N-hydroxybenzotriazolyl. In certain embodiments, -LG is pentafluorphenoxy. In certain embodiments, -LG is N-hydroxysulfosuccinimidyl. In certain embodiments, -LG is diphenylphosphinomethanethiyl. In certain embodiments, -LG is 2-diphenylphosphinophenoxy. In certain embodiments, -LG is norbornene-N-hydroxysuccinimidyl. In certain embodiments, -LG is N-hydroxyphthalimide.

In certain embodiments, -LG is pyridinoxy. In certain embodiments, -LG is nonafluoro tert-butyloxy. In certain embodiments, -LG is hexafluoro isopropyloxy.

In certain embodiments, —Y of formula (II) is as disclosed in WO2016/020373A1. Accordingly, —Y of formula (II) is selected from the group consisting of thiol, maleimide, amine, hydroxyl, carboxylic acid and derivatives, carbonate and derivatives, carbamate and derivatives, isothiocyanate, disulfide, pyridyl disulfide, methylthiosulfonyl, vinylsulfone, aldehyde, ketone, haloacetyl, selenide, azide, —NH—NH₂, —O—NH₂, a terminal alkyne, a compound of formula (z′i)

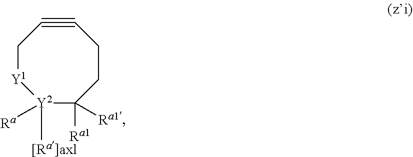

wherein
Y¹, Y² are independently C or N,
R^{a}, R^{a′}, R^{a1}, R^{a1′} are independently —H or C₁₋₆ alkyl,
ax1 is 0, if Y² is N; ax1 is 1, if Y² is C,
optionally, the pair R^{a}/R^{a1} forms a chemical bond, if Y² is C,
optionally, the pair R^{a′}/R^{a1′} are joined together with the atom to which they are attached to form a ring A′, if Y² is C, and
A′ is cyclopropyl or phenyl;

a compound of formula (z′ii)

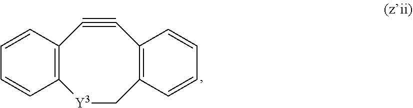

wherein
Y³ is C or N;
a compound of formula (z′iii)

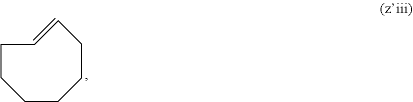

a compound of formula (z′iv),

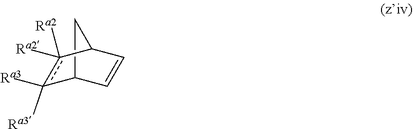

wherein
R^{a2}, R^{a2′}, R^{a3}, R^{a3′} are —H,
[symbol] indicates a single or double bond,
optionally, the pair R^{a2′}/R^{a3′} are joined together with the atoms to which they are attached to form a ring A^{1′}; and
A^{2′} is 5-membered heterocyclyl;

a compound of formula (z′v)

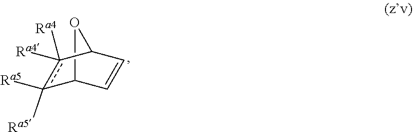

wherein
R^{a4}, R^{a4′}, R^{a5}, R^{a5′} are —H,
[symbol] indicates a single or double bond,
optionally, the pair R^{a4}/R^{a5} forms a chemical bond, optionally, the pair R^{a4′}/R^{a5′} are joined together with the atoms to which they are
attached to form a ring A^{2′}, and
A^{2′} is 5-membered heterocyclyl;

a compound of formula (z′vi)

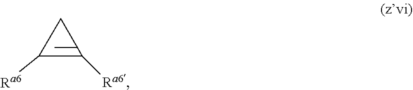

wherein
R^{a6}, R^{a6′} are either both C₁₋₆ alkyl or one of R^{a6}, R^{a6′} is —H and the other one is selected from C₁₋₆ alkyl, —COOR^{a7}, —CONHR^{a7′}, and CH₂OR^{a7″}, and
R^{a7}, R^{a7′}, R^{a7″} are independently —H or C₁₋₄ alkyl;

a compound of formula (z′vii)

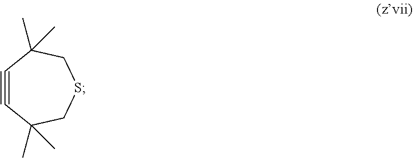

a compound of formula (z′viii)

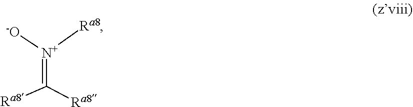

wherein
R^{a8}, R^{a8′}, R^{a8″} are independently selected from the group consisting of —H and C₁₋₄ alkyl;

a compound of formula (z′ix)

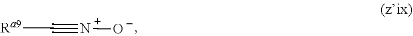

wherein
R^{a9} is —H or C₁₋₄ alkyl;

a compound of formula (z′x)

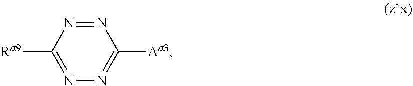

wherein
R^{a9} is selected from —COOR^{a11}, —CONHR^{a11}, and

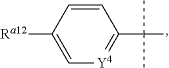

wherein
Y⁴ is C or N,
R^{a12} is selected from the group consisting of —H, —COOR^{a13}, —CONR^{a13}R^{a13′}, —CH₂NR^{a13}R^{a13′}, and —NR^{a13}COR^{a13′}, and
R^{a13}, R^{a13′} are independently selected from the group consisting of —H and C₁₋₄ alkyl,
A^{a3} is selected from the group consisting of —H, methyl, tert-butyl, —CF₃, —COOR,

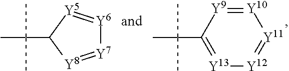

wherein
   each Y⁵, Y⁶, Y⁷, Y⁸ is independently C or N, provided that no more than 3 of Y⁵, Y⁶, Y⁷, Y⁸ are N,
   each of Y⁹, Y¹⁰, Y¹¹, Y¹², Y¹³ is either C, N, S or O, provided that no more than 4 of Y⁹, Y¹⁰, Y¹¹, Y¹², Y¹³ are N, S, or O;

a compound of formula (z′xi)

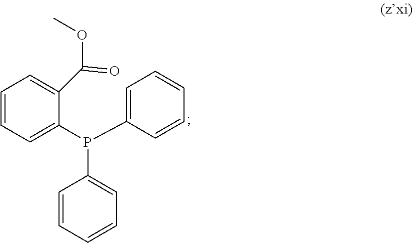 [SMILES: COC(=O)c1ccccc1P(c1ccccc1)c1ccccc1]

a compound of formula (z′xii)

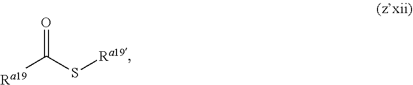

wherein
R^{a19}, R^{a19′} are independently selected from the group consisting of —H, C₁₋₆ alkyl, C₂₋₆ alkenyl, C₂₋₆ alkynyl, C₃₋₈ cycloalkyl, 3- to 10-membered heterocyclyl, 8- to 11-membered heterobicyclyl, phenyl, naphthyl, indenyl, indanyl, and tetralinyl;

a compound of formula (z′xiii)

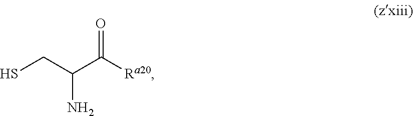

wherein
R^{a20} is selected from the group consisting of —H, C₁₋₆ alkyl, C₂₋₆ alkenyl, C₂₋₆ alkynyl, C₃₋₈ cycloalkyl, 3- to 10-membered heterocyclyl, 8- to 11-membered heterobicyclyl, phenyl, naphthyl, indenyl, indanyl, and tetralinyl;

a compound of formula (z′xiv)

R^{a22}—Ar—Y¹⁴ (z′xiv),
wherein
Ar is selected from phenyl, naphthyl, indenyl, indanyl, and tetralinyl,
Y¹⁴ is halogen, R^{a22}, R^{a23}, R^{a23′} are independently selected from the group consisting of —H, C₁₋₆ alkyl, C₂₋₆ alkenyl, C₂₋₆ alkynyl, C₃₋₈ cycloalkyl, 3- to 10-membered heterocyclyl, 8- to 11-membered heterobicyclyl, phenyl, naphthyl, indenyl, indanyl, and tetralinyl;

a compound of formula (z′xv)

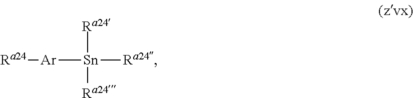

Ar is selected from phenyl, naphthyl, indenyl, indanyl, and tetralinyl,
R^{a24}, R^{a24′}, R^{a24″}, R^{a24′″} are independently selected from the group consisting of —H, C₁₋₆ alkyl, C₂₋₆ alkenyl, C₂₋₆ alkynyl, C₃₋₈ cycloalkyl, 3- to 10-membered heterocyclyl, 8- to 11-membered heterobicyclyl, phenyl, naphthyl, indenyl, indanyl, and tetralinyl;

a compound of formula (z′xvi)

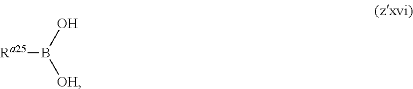

wherein
R^{a25} is selected from the group consisting of —H, C₁₋₆ alkyl, C₂₋₆ alkenyl, C₂₋₆ alkynyl, C₃₋₈ cycloalkyl, 3- to 10-membered heterocyclyl, 8- to 11-membered heterobicyclyl, phenyl, naphthyl, indenyl, indanyl, and tetralinyl;

a compound of formula (z′xvii)

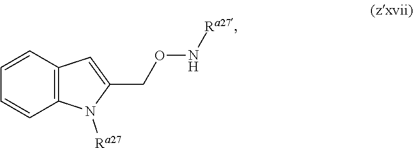

wherein
R^{a27}, R^{a27′} are independently —H or C₁₋₆ alkyl;

a compound of formula (z′xviii)

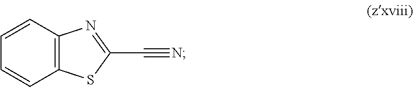 [SMILES: N#Cc1nc2ccccc2s1]

a compound of formula (z′xix)

R^{a12}—PPh₂ (z′xix),
wherein
—PPh₂ represents a group having the following formula

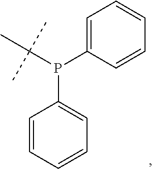

wherein the dashed line indicates attachment to the rest of the moiety of formula (z′xix), R^{a12} is selected from the group consisting of

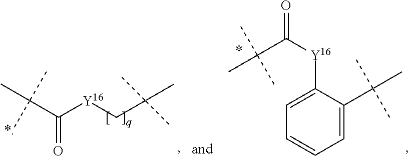

wherein
the unmarked dashed line indicates attachment to the rest of the moiety of formula (z′xix),
the dashed line with the asterisk indicates attachment to -L²-,
q is 1 or 2, and
Y¹⁶ is O or S;

a compound of formula (z′xx)

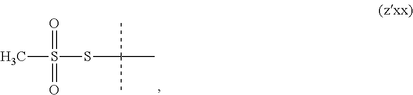

wherein the dashed line indicates attachment to -L²-; and

a compound of formula (z′xxi)

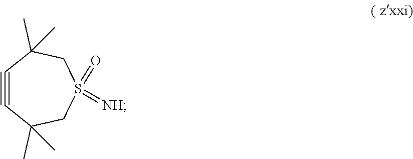

wherein the moieties of formula (z′i), (z′ii), (z′iii), (z′iv), (z′v), (z′vi), (z′vii), (z′viii), (z′ix), (z′x), (z′xi), (z′xii), (z′xiii), (z′xiv), (z′xv), (z′xvi), (z′xvii), (z′xviii) and (z′xxi) are substituted with a moiety -L²- and are optionally further substituted. It is understood that when —Y is a compound of formula (z′i), (z′ii), (z′iii), (z′iv), (z′v), (z′vi), (z′vii), (z′viii), (z′ix), (z′x), (z′xi), (z′xii), (z′xiii), (z′xiv), (z′xv), (z′xvi), (z′xvii), (z′xviii) or (z′xxi), then any hydrogen atom of said formulas may be substituted with a moiety -L²-.

In certain embodiments, Y¹ of formula (z′i) is C.

In certain embodiments, R^{a}, R^{a′}, R^{a1}, R^{a1′} of formula (z′i) are —H.

In certain embodiments, formula (z′i) is selected from the group consisting of:

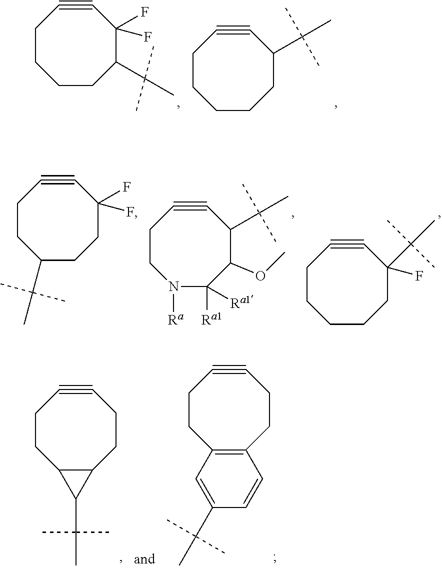

wherein the dashed line indicates attachment to -L²-, and
R^{a}, R^{a1}, R^{a1′} are used as defined in formula (z′i).

In certain embodiments, formula (z′ii) is selected from the group consisting of:

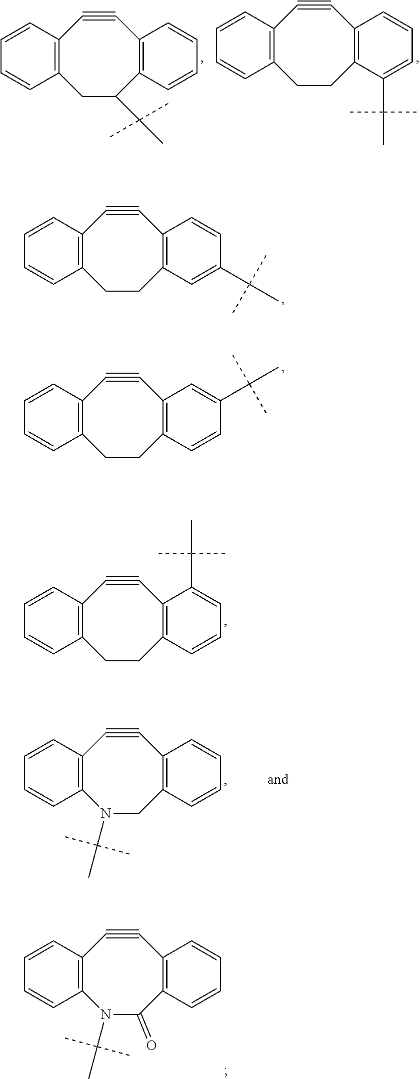

wherein the dashed line indicates attachment to -L²-.

In certain embodiments, formula (z′iii) is selected from the group consisting of

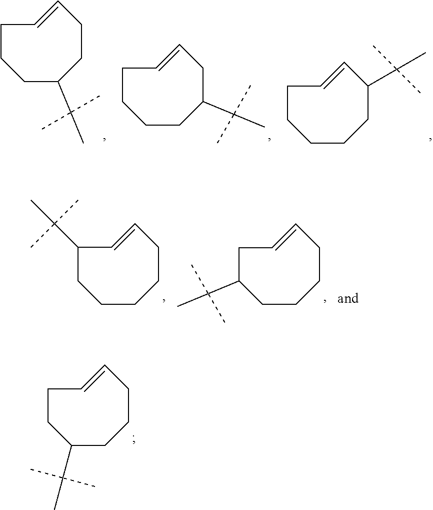

wherein the dashed line indicates attachment to -L²-.

In certain embodiments, formula (z′iv) is selected from the group consisting of:

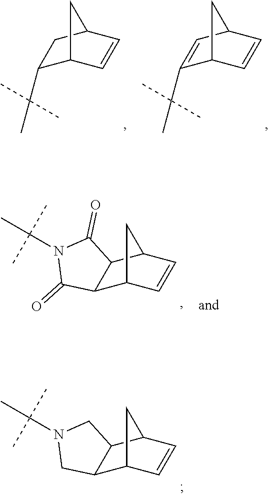

wherein the dashed line indicates attachment to -L²-.

In certain embodiments, formula (z′v) is

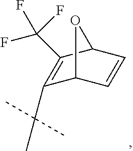

wherein the dashed line indicates attachment to -L²-.

In certain embodiments, formula (z′vi) is selected from the group consisting of:

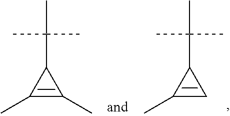

wherein the dashed line indicates attachment to -L²-.

In certain embodiments, formula (z′vii) is

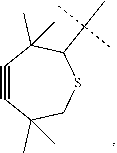

wherein the dashed line indicates attachment to -L²-.

In certain embodiments, formula (z′viii) is selected from the group consisting of:

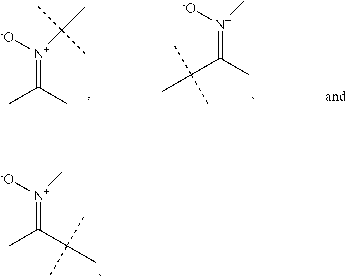

wherein the dashed line indicates attachment to -L²-.

In certain embodiments, formula (z′ix) is:

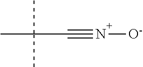

wherein the dashed line indicates attachment to -L²-.

In certain embodiments, A^{a3} of formula (z′x) is selected from the group consisting of:

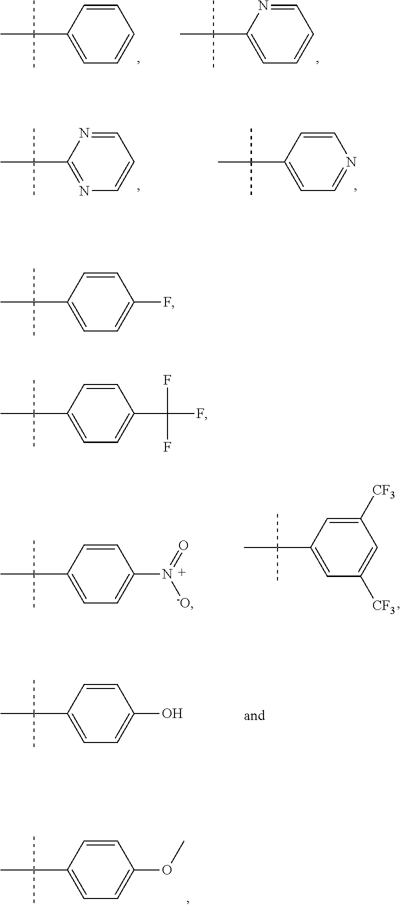

wherein the dashed line indicates attachment to the remainder of (z′x).

In certain embodiments, the moiety

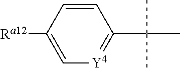

of formula (z′x) is selected from the group consisting of

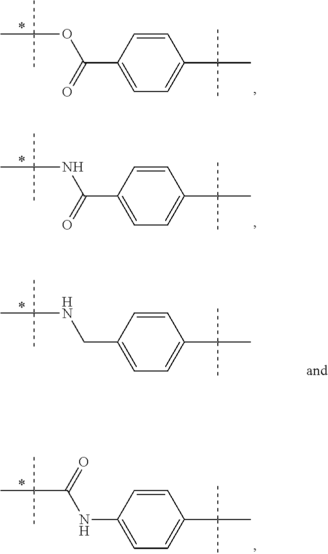

wherein
the unmarked dashed line indicates attachment to the remainder of (z′x) and
the dashed line marked with the asterisk indicates attachment to -L²-.

In certain embodiments, formula (z′xii) is selected from the group consisting of

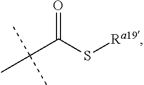

wherein the dashed line indicates attachment to -L²-, and
R^{a19′} is selected from the group consisting of H, methyl, ethyl, propyl and butyl.

In certain embodiments, formula (z′xiii) is

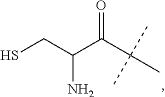

wherein the dashed line indicates attachment to -L²-.

In certain embodiments, formula (z′xiv) is

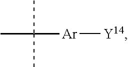

wherein the dashed line indicates attachment to -L²-,
Ar is selected from the group consisting of phenyl, naphthyl, indenyl, indanyl, and tetralinyl, and
Y¹⁴ is halogen.

In certain embodiments, formula (z′xv) is

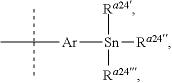

wherein the dashed line indicates attachment to -L²-,
Ar is selected from the group consisting of phenyl, naphthyl, indenyl, indanyl, and tetralinyl; and
R^{a24′}, R^{a24″}, R^{a24′″} are independently selected from the group consisting of H, methyl, ethyl, propyl and butyl.

In certain embodiments, formula (z′xvi) is

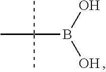

wherein the dashed line indicates attachment to -L²-.

In certain embodiments, formula (z′xvii) is

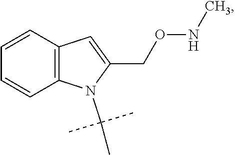

wherein the dashed line indicates attachment to -L²-.

In certain embodiments, —Y is a substituted acyl borate as disclosed in WO 2018/011266 A1. Accordingly, in certain embodiments, —Y is

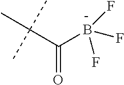

wherein the dashed line indicates attachment to -L²-.

In certain embodiments, —Y is a hydroxylamine as disclosed in WO 2018/011266 A1. Accordingly, in certain embodiments, —Y is

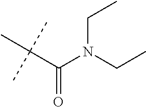

wherein the dashed line indicates attachment to -L²-.

In certain embodiments, formula (z′xxi) is

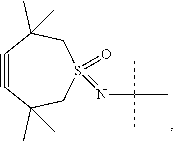

wherein the dashed line indicates attachment to -L²-.

In certain embodiments, —Y is selected from the group consisting of:

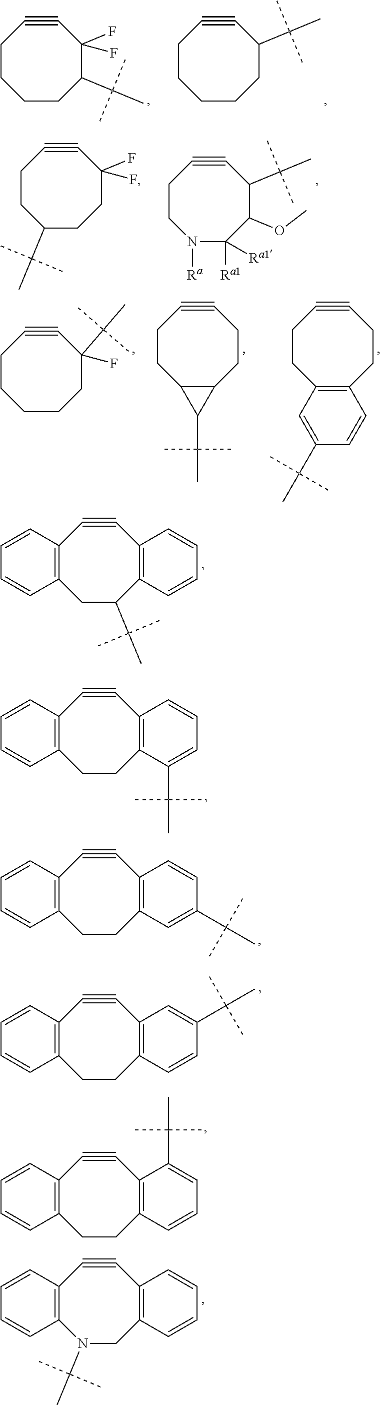

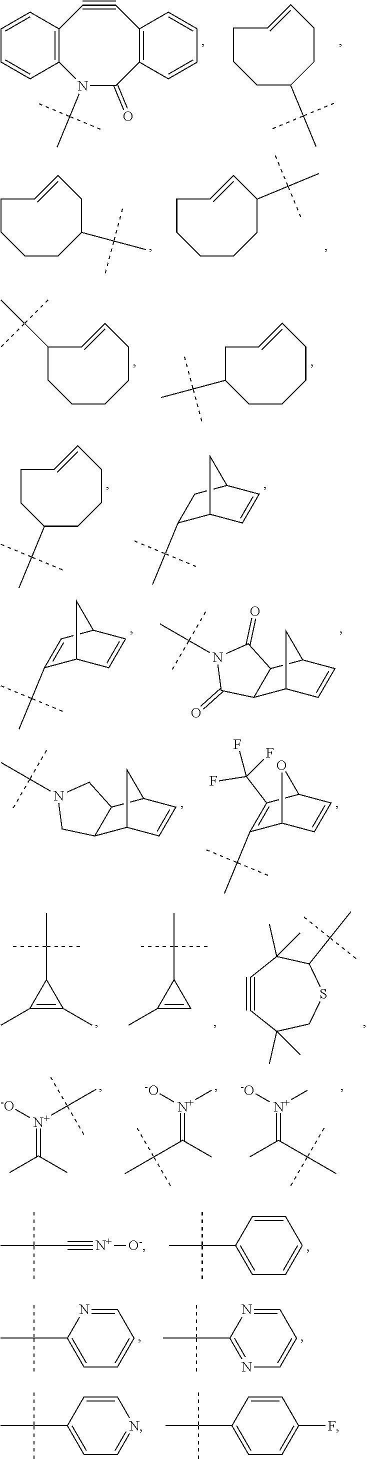

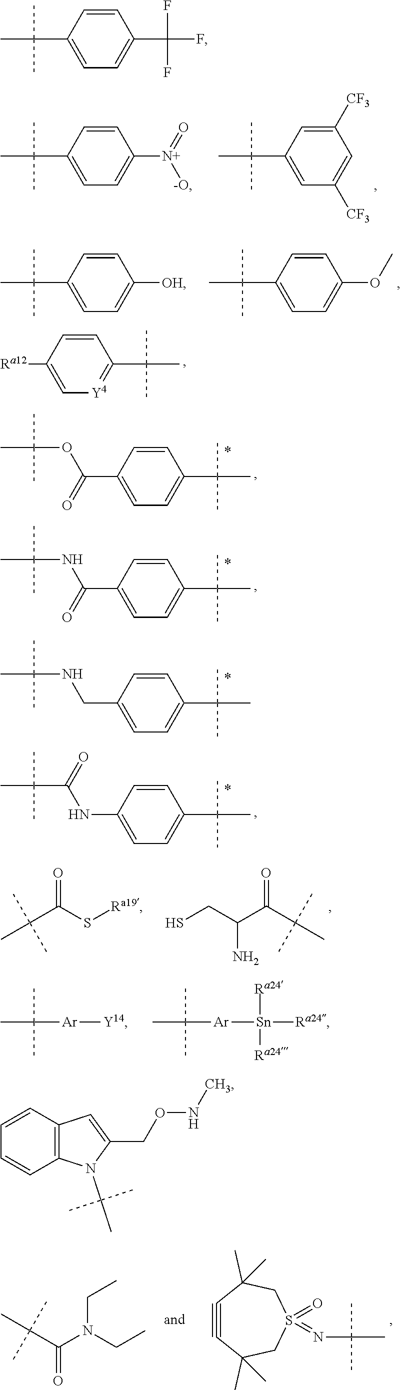

wherein the dashed line indicates attachment to -L²-;
the dashed line marked with the asterisk indicates attachment to the rest of the moiety of formula (z′xix);
R^{a}, R^{a1}, R^{a1′} are used as defined in formula (z′i), R^{a19′} is used as defined in formula (z′xii), Y⁴ is used as defined in formula (z′xiv) and R^{a24′}, R^{a24″} R^{a24′} are used as defined in formula (z′xv).

In certain embodiments, —Y is present in its protected form.

In certain embodiments, —Y is a thiol that is connected to a moiety that is used for the reversible protection of a thiol functional group. In certain embodiments, —Y is a thiol that is connected to a moiety selected from the group consisting of

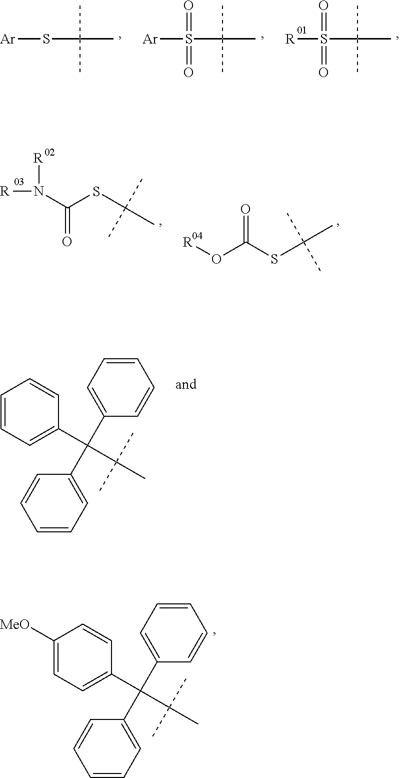

wherein
the dashed line indicates attachment to —Y;
Ar is an aromatic moiety which is optionally further substituted; and
R⁰¹, R⁰³, R⁰⁴ are independently of each other a chemical bond or is C₁₋₅₀ alkyl; C₂₋₅₀ alkenyl; or C₂₋₅₀ alkynyl, wherein C₁₋₅₀ alkyl; C₂₋₅₀ alkenyl; and C₂₋₅₀ alkynyl are optionally substituted with one or more R³, which are the same or different and wherein C₁₋₅₀ alkyl; C₂₋₅₀ alkenyl; and C₂₋₅₀ alkynyl are optionally interrupted by one or more groups selected from the group consisting of -Q-, —C(O)O—; —O—; —C(O)—; —C(O)N(R⁴)—; —S(O)₂N(R⁴)—; —S(O)N(R⁴)—; —S(0)₂—; —S(O)—; —N(R⁴)S(O)₂N(R^{4a})—; —S—; —N(R⁴)—; —OC(O)R⁴; —N(R⁴)C(O)—; —N(R⁴)S(O)₂—; —N(R⁴)S(O)—; —N(R⁴)C(O)O—; —N(R⁴)C(O)N(R^{4a})—; and —OC(O)N(R⁴R^{4a});
R⁰² is —H; C₁₋₅₀ alkyl; C₂₋₅₀ alkenyl; or C₂₋₅₀ alkynyl, wherein C₁₋₅₀ alkyl; C₂₋₅₀ alkenyl; and C₂₋₅₀ alkynyl are optionally substituted with one or more R³, which are the same or different and wherein C₁₋₅₀ alkyl; C₂₋₅₀ alkenyl; and C₂₋₅₀ alkynyl are optionally interrupted by one or more groups selected from the group consisting of -Q-, —C(O)O—; —O—; —C(O)—; —C(O)N(R⁴)—; —S(O)₂N(R⁴)—; —S(O)N(R⁴)—; —S(O)₂—; —S(O)—; —N(R⁴)S(O)₂N(R^{4a})—; —S—; —N(R⁴)—; —OC(O)R⁴; —N(R⁴)C(O)—; —N(R⁴)S(O)₂—; —N(R⁴)S(O)—; —N(R⁴)C(O)O—; —N(R⁴)C(O)N(R^{4a})—; and —OC(O)N(R⁴R^{4a});
Q is selected from the group consisting of phenyl; naphthyl; indenyl; indanyl; tetralinyl; C₃₋₁₀ cycloalkyl; 4- to 7-membered heterocyclyl; and 8- to 11-membered heterobicyclyl, wherein T is optionally substituted with one or more R³, which are the same or different;
R³ is halogen; —CN; oxo (═O); —COOR⁵; —OR⁵; —C(O)R⁵; —C(O)N(R⁵R^{5a}); —S(O)₂N(R⁵R^{5a}); —S(O)N(R⁵R^{5a}); —S(O)₂R⁵; —S(O)R⁵; —N(R⁵)S(O)₂N(R^{5a}R^{5b}); —SR⁵; —N(R⁵R^{5a}); —NO₂; —OC(O)R⁵; —N(R⁵)C(O)R^{5a}; —N(R⁵)S(O)₂R^{5a}; —N(R⁵)S(O)R^{5a}; —N(R⁵)C(O)OR^{5a}; —N(R⁵)C(O)N(R^{5a}R^{5b}); —OC(O)N(R⁵R^{5a}); or C₁₋₆ alkyl, wherein C₁₋₆ alkyl is optionally substituted with one or more halogen, which are the same or different; and
R⁴, R^{4a}, R⁵, R^{5a}, R^{5b} are independently selected from the group consisting of —H; or C₁₋₆ alkyl, wherein C₁₋₆ alkyl is optionally substituted with one or more halogen, which are the same or different.

-L¹- is connected to -D through an amide linkage. It is understood that this linkage is not reversible per se, but that in the present invention neighboring groups present in -L¹-, such as for example amide, primary amine, secondary amine and tertiary amine, render these linkages reversible.

In certain embodiments, the reagent of the present invention comprises a linker -L*- of formula (II′).

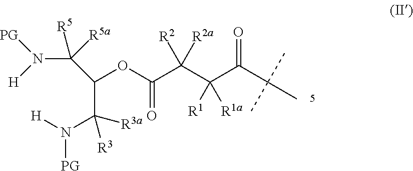

wherein the dashed line indicates attachment to -Q;
—R¹, —R^{1a}, —R², —R^{2a}, —R³, —R^{3a}, —R⁵, —R^{5a} and —PG are used as defined for formula (II);
-L*- is optionally substituted with at least one moiety -L²-Z or at least one moiety -L²-Y and optionally is further substituted; and
wherein -L²-, —Y and Z are used as defined for formula (II).

In certain embodiments, -L*- of formula (II′) is substituted with at least one moiety -L²-Y or at least one moiety -L²-Z and optionally is further substituted. In certain embodiments, -L*- of formula (II′) is substituted with at least one moiety -L²-Y or at least one moiety -L²-Z and optionally is further substituted, provided that —X³— is not —S—.

In certain embodiments, -L*- of formula (II′) is substituted with at least one moiety -L²-Y. In certain embodiments, -L*- of formula (II′) is substituted with one moiety -L²-Y. In certain embodiments, -L*- of formula (II′) is substituted with two moieties -L²-Y. In certain embodiments, -L*- of formula (II′) is substituted with three moieties -L²-Y.

In certain embodiments, -L*- of formula (II′) is substituted with at least one moiety -L²-Z. In certain embodiments, -L*- of formula (II′) is substituted with one moiety -L²-Z. In certain embodiments, -L*- of formula (II′) is substituted with two moieties -L²-Z. In certain embodiments, -L*- of formula (II′) is substituted with three moieties -L²-Z.

In certain embodiments, -L¹- is further substituted with one or more substituents.

In certain embodiments, -L¹- is not further substituted.

In certain embodiments, -L*- is further substituted with one or more substituents.

In certain embodiments, -L*- is not further substituted.

In certain embodiments, -L¹- is of formula (I-a):

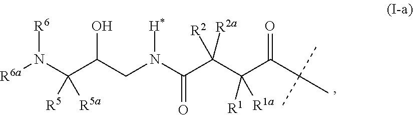

wherein
the dashed line indicates the attachment to the nitrogen of the primary or secondary amine of -D of formula (I); and
—R¹, —R^{1a}, —R², —R^{2a}, —R⁵, —R^{5a}, —R⁶ and —R^{6a} are used as defined in formula (I).

In certain embodiments, —R¹, —R^{1a}, —R², —R^{2a}, —R⁵, —R^{5a}, —R⁶ and —R^{6a} of formula (I-a) are independent of each other selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl.

In certain embodiments, —R¹, —R^{1a}, —R² are —H and —R^{2a} is —N(R¹²)C(O)H.

In certain embodiments, —R¹ of formula (I-a) is selected from the group consisting of —H, methyl, ethyl, n-propyl and iso-propyl. In certain embodiments, —R^{1a} of formula (I-a) is selected from the group consisting of —H, methyl, ethyl, n-propyl and iso-propyl. In certain embodiments, —R² of formula (I-a) is selected from the group consisting of —H, methyl, ethyl, n-propyl and iso-propyl. In certain embodiments, —R^{2a} of formula (I-a) is selected from the group consisting of —H, methyl, ethyl, n-propyl and iso-propyl. In certain embodiments, —R⁵ of formula (I-a) is selected from the group consisting of —H, methyl, ethyl, n-propyl and iso-propyl. In certain embodiments, —R^{5a} of formula (I-a) is selected from the group consisting of —H, methyl, ethyl, n-propyl and iso-propyl. In certain embodiments, —R⁶ of formula (I-a) is selected from the group consisting of —H, methyl, ethyl, n-propyl and iso-propyl. In certain embodiments, —R^{6a} of formula (Ia) is selected from the group consisting of —H, methyl, ethyl, n-propyl and iso-propyl.

In certain embodiments, —R¹ of formula (I-a) is —H. In certain embodiments, —R^{1a} of formula (I-a) is —H. In certain embodiments, —R² of formula (I-a) is —H. In certain embodiments, —R^{2a} of formula (I-a) is —H. In certain embodiments, —R⁵ of formula (I-a) is —H. In certain embodiments, —R^{5a} of formula (I-a) is —H. In certain embodiments, —R⁶ of formula (I-a) is —H. In certain embodiments, —R^{6a} of formula (I-a) is —H.

In certain embodiments, —R¹ of formula (I-a) is —H, which —H is substituted with -L²-. In certain embodiments, —R^{1a} of formula (I-a) is —H, which —H is substituted with -L²-. In certain embodiments, —R² of formula (I-a) is —H, which —H is substituted with -L²-. In certain embodiments, —R^{2a} of formula (I-a) is —H, which —H is substituted with -L²-. In certain embodiments, —R⁵ of formula (I-a) is —H, which —H is substituted with -L²-. In certain embodiments, —R^{5a} of formula (I-a) is —H, which —H is substituted with -L²-. In certain embodiments, —R⁶ of formula (I-a) is —H, which —H is substituted with -L²-. In certain embodiments, —R^{6a} of formula (I-a) is —H, which —H is substituted with -L²-.

In certain embodiments, -L¹- is of formula (I-b):

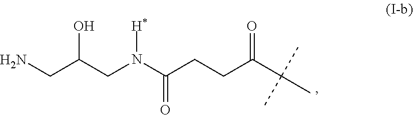

wherein
the dashed line indicates the attachment to the nitrogen of the primary or secondary amine of -D of formula (I).

In certain embodiments, -L¹- is of formula (I-c):

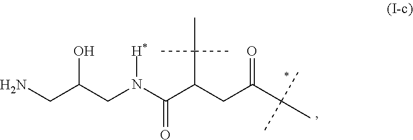

wherein
the dashed line marked with the asterisk indicates the attachment to the nitrogen of the primary or secondary amine of -D of formula (I) and the unmarked dashed line indicates attachment to -L²-; and wherein -L²- is used as defined in formula (I).

In certain embodiments, -L¹- is of formula (I-d):

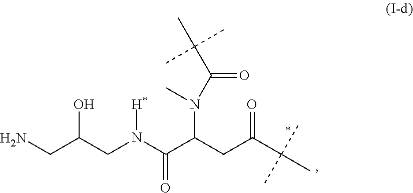

wherein the dashed line marked with the asterisk indicates the attachment to the nitrogen of the primary or secondary amine of -D of formula (I) and the unmarked dashed line indicates the attachment to -L²-, and wherein -L²- and Z are used as defined in formula (I).

In certain embodiments, all moieties -L²- of the conjugate of formula (I) are identical. In certain embodiments, the conjugate of formula (I) comprises more than one type of -L²-, such as two, three, four or five different moieties -L²-. Such more than one type of -L²- may be connected to only one type of -L¹- or may be connected to more than one type of -L¹-.

In certain embodiments, -L²- is a chemical bond.

In certain embodiments, -L²- is a spacer moiety.

In certain embodiments, -L²- is selected from the group consisting of -T′-, —C(O)O—, —O—, —C(O)—, —C(O)N(R^{y1})—, —S(O)₂N(R^{y1})—, —S(O)N(R^{y1})—, —S(O)₂—, —S(O)—, —N(R^{y1})S(O)₂N(R^{y1a})—, —S—, —N(R^{y1})—, —OC(OR^{y1})(R^{y1a})—, —N(R^{y1})C(O)N(R^{y1a})—, —OC(O)N(R^{y1})—, C₁₋₅₀ alkyl, C₂₋₅₀ alkenyl and C₂₋₅₀ alkynyl; wherein -T′-, C₁₋₅₀ alkyl, C₂₋₅₀ alkenyl and C₂₋₅₀ alkynyl are optionally substituted with one or more —R^{y2}, which are the same or different and wherein C₁₋₅₀ alkyl, C₂₋₅₀ alkenyl and C₂₋₅₀ alkynyl are optionally interrupted by one or more groups selected from the group consisting of -T′-, —C(O)O—, —O—, —C(O)—, —C(O)N(R^{y3})—, —S(O)₂N(R^{y3})—, —S(O)N(R^{y3})—, —S(O)₂—, —S(O)—, —N(R^{y3})S(O)₂N(R^{y3a})—, —S—, —N(R^{y3})—, —OC(OR^{y3})(R^{y3a})—, —N(R^{y3})C(O)N(R^{y3a})— and —OC(O)N(R^{y3})—;
—R^{y1} and —R^{y1}a are independently selected from the group consisting of —H, -T′, C₁₋₅₀ alkyl, C₂₋₅₀ alkenyl and C₂₋₅₀ alkynyl; wherein -T′, C₁₋₅₀ alkyl, C₂₋₅₀ alkenyl and C₂₋₅₀ alkynyl are optionally substituted with one or more —R^{y2}, which are the same or different, and wherein C₁₋₅₀ alkyl, C₂₋₅₀ alkenyl and C₂₋₅₀ alkynyl are optionally interrupted by one or more groups selected from the group consisting of -T′-, —C(O)O—, —O—, —C(O)—, —C(O)N(R^{y4})—, —S(O)₂N(R^{y4})—, —S(O)N(R^{y4})—, —S(O)₂—, —S(O)—, —N(R^{y4})S(O)₂N(R^{y4a})—, —S—, —N(R^{y4})—, —OC(OR^{y4})(R^{y4a})—, —N(R^{y4})C(O)N(R^{y4a})—, and —OC(O)N(R^{y4})—;
each T′ is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl and 8- to 30-membered heteropolycyclyl;
wherein each T′ is independently optionally substituted with one or more —R^{y2}, which are the same or different;
each —R^{y2} is independently selected from the group consisting of halogen, —CN, oxo (═O), —C(O)OR^{y5}, —OR^{y5}, —C(O)R^{y5}, —C(O)N(R^{y5})(R^{y5a}), —S(O)₂N(R⁵)(R^{y5a}), —S(O)N(R^{y5})(R^{y5a}), —S(O)₂R^{y5}, —S(O)R^{y5}, —N(R^{y5})S(O)₂N(R^{y5})(R^{y5a}), —SR^{y5}, —N(R^{y5})(R^{y5a}), —NO₂, —OC(O)R^{y5}, —N(R^{y5})C(O)R^{y5a}, —N(R^{y5})S(O)₂R^{y5a}, —N(R^{y5})S(O)R^{y5a}, —N(R^{y5})C(O)OR^{y5a}, —N(R^{y5})C(O)N(R^{y5})(R^{y5a}), —OC(O)N(R^{y5})(R^{y5a}), and C₁₋₆ alkyl; wherein C₁₋₆ alkyl is optionally substituted with one or more halogen, which are the same or different; and
each —R^{y3}, —R^{y3}a, —R^{y4}, —R^{y4}a, —R^{y5}, —R^{y5a} and —R^{y5b} is independently selected from the group consisting of —H and C₁₋₆ alkyl; wherein C₁₋₆ alkyl is optionally substituted with one or more halogen, which are the same or different.

In certain embodiments, -L²- is selected from the group consisting of -T′-, —C(O)O—, —O—, —C(O)—, —C(O)N(R^{y1})—, —S(O)₂N(R^{y1})—, —S(O)N(R^{y1})—, —S(O)₂—, —S(O)—, —N(R^{y1})S(O)₂N(R^{y1a})—, —S—, —N(R^{y1})—, —OC(OR^{y1})(R^{y1a})—, —N(R^{y1})C(O)N(R^{y1a})—, —OC(O)N(R^{y1})—, C₁₋₅₀ alkyl, C₂₋₅₀ alkenyl, and C₂₋₅₀ alkynyl; wherein -T′-, C₁₋₂₀ alkyl, C₂₋₂₀ alkenyl, and C₂₋₂₀ alkynyl are optionally substituted with one or more —R^{y2}, which are the same or different and wherein C₁₋₂₀ alkyl, C₂₋₂₀ alkenyl, and C₂₋₂₀ alkynyl are optionally interrupted by one or more groups selected from the group consisting of -T′-, —C(O)O—, —O—, —C(O)—, —C(O)N(R^{y3})—, —S(O)₂N(R^{y3})—, —S(O)N(R^{y3})—, —S(O)₂—, —S(O)—, —N(R^{y3})S(O)₂N(R^{y3a})—, —S—, —N(R^{y3})—, —OC(OR^{y3})(R^{y3a})—, —N(R^{y3})C(O)N(R^{y3a})—, and —OC(O)N(R^{y3})—;
—R^{y1} and —R^{y1a} are independently selected from the group consisting of —H, -T′, C₁₋₁₀ alkyl, C₂₋₁₀ alkenyl, and C₂₋₁₀ alkynyl; wherein -T′, C₁₋₁₀ alkyl, C₂₋₁₀ alkenyl, and C₂₋₁₀ alkynyl are optionally substituted with one or more —R^{y2}, which are the same or different, and wherein C₁₋₁₀ alkyl, C₂₋₁₀ alkenyl, and C₂₋₁₀ alkynyl are optionally interrupted by one or more groups selected from the group consisting of -T′-, —C(O)O—, —O—, —C(O)—, —C(O)N(R^{y4})—, —S(O)₂N(R^{y4})—, —S(O)N(R^{y4})—, —S(O)₂—, —S(O)—, —N(R^{y4})S(O)₂N(R^{y4a})—, —S—, —N(R^{y4})—, —OC(OR^{y4})(R^{y4a})—, —N(R^{y4})C(O)N(R^{y4a})—, and —OC(O)N(R^{y4})—;
each T′ is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl; wherein each T′ is independently optionally substituted with one or more —R^{y2}, which are the same or different;

—R^{y2} is selected from the group consisting of halogen, —CN, oxo (═O), —C(O)OR^{y5}, —OR^{y5}, —C(O)R^{y5}, —C(O)N(R^{y5})(R^{y5a}), —S(O)₂N(R^{y5})(R^{y5a}), —S(O)N(R^{y5})(R^{y5a}), —S(O)₂R^{y5}, —S(O)R^{y5}, —N(R^{y5})S(O)₂N(R^{y5a})(R^{y5b}), —SR^{y5}, —N(R^{y5})(R^{y5a}), —NO₂, —OC(O)R^{y5}, —N(R^{y5})C(O)R^{y5a}, —N(R^{y5})S(O)₂R^{y5a}, —N(R^{y5})S(O)R^{y5a}, —N(R^{y5})C(O)OR^{y5a}, —N(R^{y5})C(O)N(R^{y5a})(R^{y5b}), —OC(O)N(R^{y5})(R^{y5a}) and C₁₋₆ alkyl; wherein C₁₋₆ alkyl is optionally substituted with one or more halogen, which are the same or different; and

each —R^{y3}, —R^{y3}a, —R^{y4}, —R^{y4}a, —R^{y5}, —R^{y5a} and —R^{y5b} is independently selected from the group consisting of —H and C₁₋₆ alkyl; wherein C₁₋₆ alkyl is optionally substituted with one or more halogen, which are the same or different.

In certain embodiments, -L²- is selected from the group consisting of -T′-, —C(O)O—, —O—, —C(O)—, —C(O)N(R^{y1})—, —S(O)₂N(R^{y1})—, —S(O)N(R^{y1})—, —S(O)₂—, —S(O)—, —N(R^{y1})S(O)₂N(R^{y1a})—, —S—, —N(R^{y1})—, —OC(OR^{y1})(R^{y1a})—, —N(R^{y1})C(O)N(R^{y1a})—, —OC(O)N(R^{y1})—, C₁₋₅₀ alkyl, C₂₋₅₀ alkenyl, and C₂₋₅₀ alkynyl; wherein -T′-, C₁₋₅₀ alkyl, C₂₋₅₀ alkenyl, and C₂₋₅₀ alkynyl are optionally substituted with one or more —R^{y2}, which are the same or different and wherein C₁₋₅₀ alkyl, C₂₋₅₀ alkenyl, and C₂₋₅₀ alkynyl are optionally interrupted by one or more groups selected from the group consisting of -T′-, —C(O)O—, —O—, —C(O)—, —C(O)N(R^{y3})—, —S(O)₂N(R^{y3})—, —S(O)N(R^{y3})—, —S(O)₂—, —S(O)—, —N(R^{y3})S(O)₂N(R^{y3a})—, —S—, —N(R^{y3})—, —OC(OR^{y3})(R^{y3a})—, —N(R^{y3})C(O)N(R^{y3a})—, and —OC(O)N(R^{y3})—;

—R^{y1} and —R^{y1}a are independently selected from the group consisting of —H, -T′, C₁₋₁₀ alkyl, C₂₋₁₀ alkenyl and C₂₋₁₀ alkynyl;

each T′ is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl and 8- to 30-membered heteropolycyclyl; each —R^{y2} is independently selected from the group consisting of halogen, and C₁₋₆ alkyl; and each —R^{y3}, —R^{y3}a, —R^{y4}, —R^{y4}a, —R^{y5}, —R^{y5a} and —R^{y5b} is independently selected from the group consisting of —H and C₁₋₆ alkyl; wherein C₁₋₆ alkyl is optionally substituted with one or more halogen, which are the same or different.

In certain embodiments, -L²- is a C₁₋₂₀ alkyl chain, which is optionally interrupted by one or more groups independently selected from the group consisting of —O—, -T′- and —C(O)N(R^{y1})—; and which C₁₋₂₀ alkyl chain is optionally substituted with one or more groups independently selected from the group consisting of —OH, -T′ and —C(O)N(R^{y6}R^{y6a}); wherein —R^{y1}, —R^{y6}, —R^{y6a} are independently selected from the group consisting of H and C₁₋₄ alkyl and wherein T′ is selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl and 8- to 30-membered heteropolycyclyl.

In certain embodiments, -L²- has a molecular weight ranging from 14 g/mol to 750 g/mol.

In certain embodiments, -L²- comprises a moiety selected from the group consisting of.

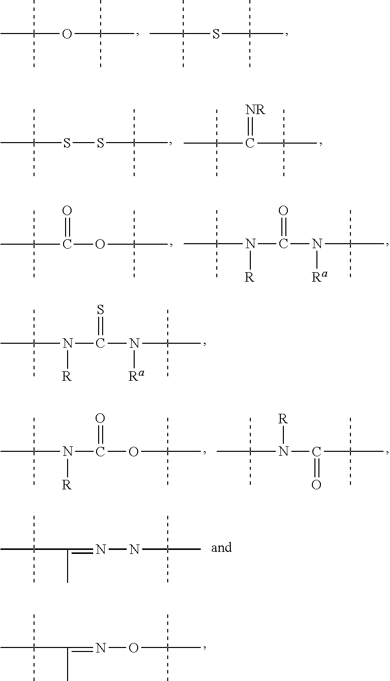

wherein dashed lines indicate attachment to -L¹-, the remainder of -L²- or Z, respectively; and —R and —R^{a} are independently selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl.

In certain embodiments, -L²- comprises a moiety selected from

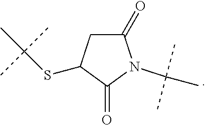

In certain embodiments, -L²- has a chain length of 1 to 20 atoms.

As used herein, the term “chain length” with regard to the moiety -L²- refers to the number of atoms of -L²- present in the shortest connection between -L¹- and —Z.

In general, -L²- may be attached to -L¹- or -L*- at any position where one hydrogen given by —R¹, —R^{1a}, —R², —R^{2a}, —R³, —R^{3a}, —R⁴, —R^{4a}, —R⁵, —R^{5a}, —R⁶, —R^{6a}, —R⁷, —R⁸, —R^{8a}, —R⁹, —R¹⁰, —R¹¹, —R^{11a}, —R^{11b}, —R¹², —R^{12a}, —R¹³, —R¹⁴, —R^{14a}, —R¹⁵, —R^{15a} and —R^{15b} of formula (I) or (II) is replaced by -L²-.

In certain embodiments, one hydrogen given by —R¹ of formula (I) or (II) is replaced by -L²-. In certain embodiments, one hydrogen given by —R^{1a} of formula (I) or (II) is replaced by -L²-. In certain embodiments, one hydrogen given by —R² of formula (I) or (II) is replaced by -L²-. In certain embodiments, one hydrogen given by —R^{2a} of formula (I) or (II) is replaced by -L²-. In certain embodiments, one hydrogen given by —R³ of formula (I) or (II) is replaced by -L²-. In certain embodiments, one hydrogen given by —R^{3a} of formula (I) or (II) is replaced by -L²-. In certain embodiments, one hydrogen given by —R⁴ of formula (I) or (II) is replaced by -L²-. In certain embodiments, one hydrogen given by —R^{4a} of formula (I) or (II) is replaced by -L²-. In certain embodiments, one hydrogen given by —R⁵ of formula (I) or (II) is replaced by -L²-. In certain embodiments, one hydrogen given by —R^{5a} of formula (I) or (II) is replaced by -L²-. In certain embodiments, one hydrogen given by —R⁶ of formula (I) or (II) is replaced by -L²-. In certain embodiments, one hydrogen given by —R^{6a} of formula (I) or (II) is replaced by -L²-. In certain embodiments, one hydrogen given by —R⁷ of formula (I) or (II) is replaced by -L²-. In certain embodiments, one hydrogen given by —R⁸ of formula (I) or (II) is replaced by -L²-. In certain embodiments, one hydrogen given by —R^{8a} of formula (I) or (II) is replaced by -L²-. In certain embodiments, one hydrogen given by —R⁹ of formula (I) or (II) is replaced by -L²-. In certain embodiments, one hydrogen given by —R¹⁰ of formula (I) or (II) is replaced by -L²-. In certain embodiments, one hydrogen given by —R¹¹ of formula (T) or (TT) is replaced by -L²-. In certain embodiments, one hydrogen given by —R^{11a} of formula (I) or (II) is replaced by -L²-. In certain embodiments, one hydrogen given by —R^{11b} of formula (I) or (II) is replaced by -L²-. In certain embodiments, one hydrogen given by —R¹² of formula (I) or (II) is replaced by -L²-. In certain embodiments, one hydrogen given by —R^{12a} of formula (I) or (II) is replaced by -L²-. In certain embodiments, one hydrogen given by —R^{12b} of formula (I) or (II) is replaced by -L²-. In certain embodiments, one hydrogen given by —R¹³ of formula (I) or (II) is replaced by -L²-. In certain embodiments, one hydrogen given by —R¹⁴ of formula (I) or (II) is replaced by -L²-. In certain embodiments, one hydrogen given by —R^{14a} of formula (I) or (II) is replaced by -L²-. In certain embodiments, one hydrogen given by —R¹⁵ of formula (I) or (II) is replaced by -L²-. In certain embodiments, one hydrogen given by —R^{15a} of formula (I) or (II) is replaced by -L²-. In certain embodiments, one hydrogen given by —R^{15b} of formula (I) or (II) is replaced by -L²-.

In certain embodiments, the linkage between Z and -L²- is a stable linkage.

In certain embodiments, Z is a C₈₋₂₄ alkyl moiety.

In certain embodiments, Z is water-soluble.

In certain embodiments, Z is a water-soluble polymeric moiety.

If Z is a water-soluble polymeric moiety, such polymeric moiety has a molecular weight ranging from and including 1 kDa to 1000 kDa. In certain embodiments, Z has a molecular weight ranging from and including 5 kDa to 1000 kDa. In certain embodiments, Z has a molecular weight ranging from and including 5 kDa to 500 kDa. In certain embodiments, Z has a molecular weight ranging from and including 10 kDa to 250 kDa. In certain embodiments, Z has a molecular weight ranging from and including 10 kDa to 150 kDa. In certain embodiments, Z has a molecular weight ranging from and including 12 kDa to 100 kDa. In certain embodiments, Z has a molecular weight ranging from and including 15 kDa to 80 kDa. In certain embodiments, Z has a molecular weight ranging from and including 10 kDa to 80 kDa.

In certain embodiments, Z has a molecular weight of about 80 kDa. In certain embodiments, Z has a molecular weight of about 70 kDa. In certain embodiments, Z has a molecular weight of about 60 kDa. In certain embodiments, Z has a molecular weight of about 50 kDa. In certain embodiments, Z has a molecular weight of about 40 kDa. In certain embodiments, Z has a molecular weight of about 30 kDa. In certain embodiments, Z has a molecular weight of about 20 kDa. In certain embodiments, Z has a molecular weight of about 10 kDa. In certain embodiments, Z has a molecular weight of about 5 kDa.

In certain embodiments, Z is a water-soluble polymeric moiety comprising a polymer selected from the group consisting of 2-methacryloyl-oxyethyl phosphoyl cholins, poly(acrylic acids), poly(acrylates), poly(acrylamides), poly(alkyloxy) polymers, poly(amides), poly(amidoamines), poly(amino acids), poly(anhydrides), poly(aspartamides), poly(butyric acids), poly(glycolic acids), polybutylene terephthalates, poly(caprolactones), poly(carbonates), poly(cyanoacrylates), poly(dimethylacrylamides), poly(esters), poly(ethylenes), poly(ethyleneglycols), poly(ethylene oxides), poly(ethyl phosphates), poly(ethyloxazolines), poly(glycolic acids), poly(hydroxyethyl acrylates), poly(hydroxyethyl-oxazolines), poly(hydroxymethacrylates), poly(hydroxypropylmethacrylamides), poly(hydroxypropyl methacrylates), poly(hydroxypropyloxazolines), poly(iminocarbonates), poly(lactic acids), poly(lactic-co-glycolic acids), poly(methacrylamides), poly(methacrylates), poly(methyloxazolines), poly(organophosphazenes), poly(ortho esters), poly(oxazolines), poly(propylene glycols), poly(siloxanes), poly(urethanes), poly(vinyl alcohols), poly(vinyl amines), poly(vinylmethylethers), poly(vinylpyrrolidones), silicones, celluloses, carbomethyl celluloses, hydroxypropyl methylcelluloses, chitins, chitosans, dextrans, dextrins, gelatins, hyaluronic acids and derivatives, functionalized hyaluronic acids, mannans, pectins, rhamnogalacturonans, starches, hydroxyalkyl starches, hydroxyethyl starches and other carbohydrate-based polymers, xylans and copolymers thereof.

In certain embodiments, Z is a water-soluble polymeric moiety comprising a protein, such as a protein selected from the group consisting of carboxyl-terminal peptide of the chorionic gonadotropin as described in US 2012/0035101 A1 which are herewith incorporated by reference; albumin; XTEN sequences as described in WO 2011123813 A2 which are herewith incorporated by reference; proline/alanine random coil sequences as described in WO 2011/144756 A1 which are herewith incorporated by reference; proline/alanine/serine random coil sequences as described in WO 2008/155134 A1 and WO 2013/024049 A1 which are herewith incorporated by reference; and Fc-fusion proteins.

In certain embodiments, Z is a polysarcosine.

In certain embodiments, Z comprises poly(N-methylglycine).

In certain embodiments, Z comprises a random coil protein moiety.

In certain embodiments, such random coil protein moiety comprises at least 25 amino acid residues and at most 2000 amino acids. In certain embodiments, such random coil protein moiety comprises at least 30 amino acid residues and at most 1500 amino acid residues. In certain embodiments, such random coil protein moiety comprises at least 50 amino acid residues and at most 500 amino acid residues.

In certain embodiments, Z comprises a random coil protein moiety of which at least 80%, in certain embodiments at least 85%, in certain embodiments at least 90%, in certain embodiments at least 95%, in certain embodiments at least 98% and in certain embodiments at least 99% of the total number of amino acids forming said random coil protein moiety are selected from alanine and proline. In certain embodiments, at least 10%, but less than 75%, in certain embodiments less than 65% of the total number of amino acid residues of such random coil protein moiety are proline residues. In certain embodiments, such random coil protein moiety is as described in WO 2011/144756 A1, which is hereby incorporated by reference in its entirety.

In certain embodiments, Z comprises a random coil protein moiety of which at least 80%, in certain embodiments at least 85%, in certain embodiments at least 90%, in certain embodiments at least 95%, in certain embodiments at least 98% and in certain embodiments at least 99% of the total number of amino acids forming said random coil protein moiety are selected from alanine, serine and proline. In certain embodiments, at least 4%, but less than 40% of the total number of amino acid residues of such random coil protein moiety are proline residues. In certain embodiments, such random coil protein moiety is as described in WO 2008/155134 A1, which is hereby incorporated by reference in its entirety.

In certain embodiments, Z comprises a random coil protein moiety of which at least 80%, in certain embodiments at least 85%, in certain embodiments at least 90%, in certain embodiments at least 95%, in certain embodiments at least 98% and in certain embodiments 99% of the total number of amino acids forming said random coil protein moiety are selected from alanine, glycine, serine, threonine, glutamate and proline. In certain embodiments, such random coil protein moiety is as described in WO 2010/091122 A1 which is hereby incorporated by reference.

In certain embodiments, Z is a hyaluronic acid-based polymer.

In certain embodiments, Z is a polymeric moiety as disclosed in WO 2013/024047 A1 which is herewith incorporated by reference.

In certain embodiments, Z is a polymeric moiety as disclosed in WO 2013/024048 A1 which is herewith incorporated by reference.

In certain embodiments, Z is a PEG-based polymer, such as linear, branched or multi-arm PEG-based polymer.

In certain embodiments, Z is a linear PEG-based polymer.

In certain embodiments, Z is a branched C₈₋₂₄ alkyl having one, two, three, four, five or six branching points. In certain embodiments, Z is a branched C₈₋₂₄ alkyl having one, two or three branching points. In certain embodiments, Z is a branched C₈₋₂₄ alkyl having one branching point. In certain embodiments, Z is a branched C₈₋₂₄ alkyl having two branching points. In certain embodiments, Z is a branched C₈₋₂₄ alkyl having three branching points.

In certain embodiments, Z is a branched polymer. In certain embodiments, Z is a branched polymer having one, two, three, four, five or six branching points. In certain embodiments, Z is a branched polymer having one, two or three branching points. In certain embodiments, Z is a branched polymer having one branching point. In certain embodiments, Z is a branched polymer having two branching points. In certain embodiments, Z is a branched polymer having three branching points.

In certain embodiments, a branching point is selected from the group consisting of —N<, —CH< and >C<.

In certain embodiments, such branched moiety Z is PEG-based.

In certain embodiments, Z is a multi-arm PEG-based polymer.

In certain embodiments, Z is a multi-arm PEG-based polymer having at least 2 PEG-based arms, such as 2, 3, 4, 5, 6, 7, or 8 PEG-based arms.

In certain embodiments, Z is a branched PEG-based polymer comprising at least 10% PEG, has one branching point and two PEG-based polymer arms and has a molecular weight of about 40 kDa. Accordingly, each of the two PEG-based polymer arms has a molecular weight of about 20 kDa. In certain embodiments, the branching point is —CH<.

In certain embodiments, Z is a branched PEG-based polymer comprising at least 10% PEG, has three branching points and four PEG-based polymer arms and has a molecular weight of about 40 kDa. Accordingly, each of the four PEG-based polymer arms has a molecular weight of about 10 kDa. In certain embodiments, each of the three branching points is —CH<.

In certain embodiments, Z is water-insoluble.

In certain embodiments, Z is a water-insoluble polymeric moiety.

In certain embodiments, Z is a water-insoluble polymeric moiety comprising a polymer selected from the group consisting of 2-methacryloyl-oxyethyl phosphoyl cholins, poly(acrylic acids), poly(acrylates), poly(acrylamides), poly(alkyloxy) polymers, poly(amides), poly(amidoamines), poly(amino acids), poly(anhydrides), poly(aspartamides), poly(butyric acids), poly(glycolic acids), polybutylene terephthalates, poly(caprolactones), poly(carbonates), poly(cyanoacrylates), poly(dimethylacrylamides), poly(esters), poly(ethylenes), poly(ethyleneglycols), poly(ethylene oxides), poly(ethyl phosphates), poly(ethyloxazolines), poly(glycolic acids), poly(hydroxyethyl acrylates), poly(hydroxyethyl-oxazolines), poly(hydroxymethacrylates), poly(hydroxypropylmethacrylamides), poly(hydroxypropyl methacrylates), poly(hydroxypropyloxazolines), poly(iminocarbonates), poly(lactic acids), poly(lactic-co-glycolic acids), poly(methacrylamides), poly(methacrylates), poly(methyloxazolines), poly(organophosphazenes), poly(ortho esters), poly(oxazolines), poly(propylene glycols), poly(siloxanes), poly(urethanes), poly(vinyl alcohols), poly(vinyl amines), poly(vinylmethylethers), poly(vinylpyrrolidones), silicones, celluloses, carbomethyl celluloses, hydroxypropyl methylcelluloses, chitins, chitosans, dextrans, dextrins, gelatins, hyaluronic acids and derivatives, functionalized hyaluronic acids, mannans, pectins, rhamnogalacturonans, starches, hydroxyalkyl starches, hydroxyethyl starches and other carbohydrate-based polymers, xylans and copolymers thereof.

In certain embodiments, Z is a hydrogel.

In certain embodiments, Z is a PEG-based or hyaluronic acid-based hydrogel. In certain embodiments, Z is a PEG-based hydrogel. In certain embodiments, Z is a hyaluronic acid-based hydrogel.

In certain embodiments, Z is a hydrogel as described in WO 2006/003014 A2, WO 2011/012715 A1 or WO 2014/056926 A1, which are herewith incorporated by reference in their entirety.

In certain embodiments, Z is a hydrogel as disclosed in WO 2013/036847 A1. In particular, in certain embodiments, Z is a hydrogel produced by a method comprising the step of reacting at least a first reactive polymer with a cleavable crosslinker compound, wherein said cleavable crosslinker compound comprises a first functional group —Y¹ that reacts with the first reactive polymer and further comprises a moiety that is cleaved by elimination under physiological conditions wherein said moiety comprises a second functional group —Y² that reacts with a second reactive polymer. In certain embodiments, the cleavable crosslinker compound is of formula (PL-1):

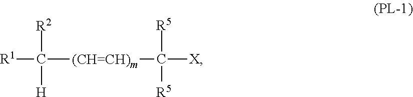

wherein
m is 0 or 1;
—X comprises a functional group capable of connecting to a reactive polymer that is amenable to elimination under physiological conditions and said second functional group —Y²;
at least one of —R¹, —R² and —R⁵ comprises said first functional group —Y¹ capable of connecting to a polymer;
one and only one of —R¹ and —R² is selected from the group consisting of —H, alkyl, arylalkyl, and heteroarylalkyl;
optionally, —R¹ and —R² may be joined to form a 3- to 8-membered ring;
at least one or both of —R¹ and —R² is independently selected from the group consisting of —CN, —NO₂, aryl, heteroaryl, alkenyl, alkynyl, —COR³, —SOR³, —SO₂R³ and —SR⁴;
—R³ is selected from the group consisting of —H, alkyl, aryl, arylalkyl, heteroaryl, heteroarylalkyl, —OR⁹ and —NR⁹₂;
—R⁴ is selected from the group consisting of alkyl, aryl, arylalkyl, heteroaryl and heteroarylalkyl;
each —R⁵ is independently selected from the group consisting of —H, alkyl, alkenylalkyl, alkynylalkyl, (OCH₂CH₂)ₚO-alkyl with p being an integer ranging from 1 to 1000, aryl, arylalkyl, heteroaryl and heteroarylalkyl;
each —R⁹ is independently selected from the group consisting of —H and alkyl or both —R⁹ together with the nitrogen to which they are attached form a heterocyclic ring;
and wherein the moiety of formula (PL-1) is optionally further substituted.

The following paragraphs describe such hydrogel in more detail.

In certain embodiments, —X of formula (PL-1) is selected from the group consisting of succinimidyl carbonate, sulfosuccinimidyl carbonate halides, thioethers, esters, nitrophenyl carbonate, chloroformate, fluoroformate, optionally substituted phenols and formula (PL-2):

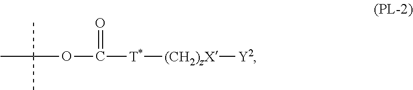

wherein
the dashed line indicates attachment to the remainder of formula (PL-1);
T*- is selected from the group consisting of —O—, —S— and —NR⁶—;
z is an integer selected from the group consisting of 1, 2, 3, 4, 5 and 6;
—X′— is absent or is selected from the group consisting of —OR⁷— and —SR⁷—;
—Y² is a functional group capable of connecting with a reactive polymer;
—R⁶ is selected from the group consisting of —H, alkyl, aryl, heteroaryl, arylalkyl, and heteroarylalkyl; and
—R⁷ is selected from the group consisting of alkylene, phenylene and (OCH₂CH₂)ₚ, with p being an integer ranging from 1 to 1000.

In certain embodiments, —X of formula (PL-1) comprises an activated carbonate such as succinimidyl carbonate, sulfosuccinimidyl carbonate, or nitrophenyl carbonate. In certain embodiments, —X of formula (PL-1) comprises a carbonyl halide such as O(C═O)C₁ or O(C═O)F. In certain embodiments, —X of formula (PL-1) has the formula (PL-2). In certain embodiments, —X of formula (PL-1) is OR⁷ or SR⁷, wherein R⁷ is optionally substituted alkylene, optionally substituted phenylene or (OCH₂CH₂)ₚ, wherein p is 1 to 1000.

In certain embodiments, p of formula (PL-2) is an integer ranging from 1 to 100. In certain embodiments, p of formula (PL-2) is an integer ranging from 1 to 10.

In certain embodiments, —Y¹ of formula (PL-1) and —Y² of formula (PL-2) independently comprise N₃, NH₂, NH—CO₂^{t}Bu, SH, S^{t}Bu, maleimide, CO₂H, CO₂^{t}Bu, 1,3-diene, cyclopentadiene, furan, alkyne, cyclooctyne, acrylate or acrylamide, wherein ^{t}Bu is tert-butyl, and wherein when one of —Y¹ or —Y² comprises N₃ the other does not comprise alkyne or cyclooctyne; when one of —Y¹ or —Y² comprises SH the other does not comprise maleimide, acrylate or acrylamide; when one of —Y¹ or —Y² comprises NH₂ the other does not comprise CO₂H; and when one of —Y¹ or —Y² comprises 1,3-diene or cyclopentadiene the other does not comprise furan.

In certain embodiments, the cleavable crosslinker compound is of formula (PL-3):

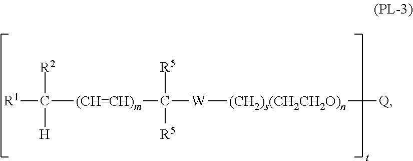

wherein
m is 0 or 1;
n is an integer selected from 1 to 1000;
s is 0, 1 or 2;
t is selected from the group consisting of 2, 4, 8, 16 and 32;
—W— is selected from the group consisting of —O(C═O)O—, —O(C═O)NH—, —O(C═O)S—, —O(C═O)NR⁶CH₂O— and —O(C═O)NR⁶S—;
Q is a core group having a valency=t; which connects the multiple arms of the cleavable crosslinking compound;
wherein t is an integer selected from 2, 4, 8, 16 and 32; and
wherein —R¹, —R² and —R⁵ are defined as in formula (PL-1).

In certain embodiments, t of formula (PL-3) is 2. In certain embodiments, t of formula (PL-3) is 4. In certain embodiments, t of formula (PL-3) is 8. In certain embodiments, t of formula (PL-3) is 16. In certain embodiments, t of formula (PL-3) is 32.

In certain embodiments, -Q of formula (PL-3) has a structure selected from the group consisting of:

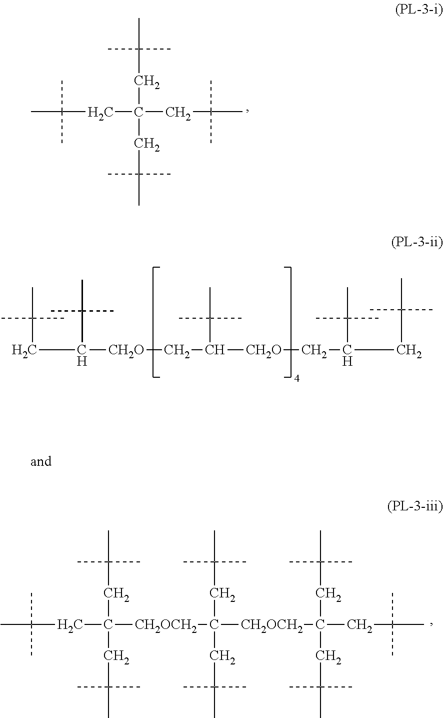

wherein the dashed lines indicate attachment to the remainder of the cleavable crosslinker compound.

In certain embodiments, -Q of formula (PL-3) has the structure of (PL-3-i). In certain embodiments, -Q of formula (PL-3) has the structure of (PL-3-ii). In certain embodiments, -Q of formula (PL-3) has the structure of (PL-3-iii).

In certain embodiments, the cleavable crosslinker compound is of formula (PL-3), wherein m is 0, n is approximately 100, s is 0, t is 4, —W— is —O(C═O)NH—, -Q has the structure of (PL-3i), —R² is H, one —R⁵ is —H and the other —R⁵ is (CH₂)₅N₃, and —R¹ is (4-chlorophenyl)SO₂, phenyl substituted with —SO₂, morpholino-SO₂, or —CN.

In certain embodiments, —Y¹ of formula (PL-3) comprises N₃, NH₂, NH—CO₂^{t}Bu, SH, S^{t}Bu, maleimide, CO₂H, CO₂^{t}Bu, 1,3-diene, cyclopentadiene, furan, alkyne, cyclooctyne, acrylate or acrylamide, wherein ^{t}Bu is tert-butyl.

In certain embodiments, each —Y¹ of formula (PL-1) or (PL-3) and —Y² of formula (PL-2) independently comprises N₃, NH₂, NH—CO₂^{t}Bu, SH, S^{t}Bu, maleimide, CO₂H, CO₂^{t}Bu, 1,3-diene, cyclopentadiene, furan, alkyne, cyclooctyne, acrylate or acrylamide.

In certain embodiments, one of —Y¹ and —Y² is azide and the other is a reactive functional group selected from the group consisting of acetylene, cyclooctyne, and maleimide. In certain embodiments, one of —Y¹ and —Y² is thiol and the other is a reactive functional group selected from the group consisting of maleimide, acrylate, acrylamide, vinylsulfone, vinylsulfonamide, and halocarbonyl. In certain embodiments, one of —Y¹ and —Y² is amine and the other is a selective reactive functional group selected from carboxylic acid and activated carboxylic acid. In certain embodiments, one of —Y¹ and —Y² is maleimide and the other is a selective reactive functional group selected from the group consisting of 1,3-diene, cyclopentadiene, and furan.

In certain embodiments, the first and any second polymer is selected from the group consisting of homopolymeric or copolymeric polyethylene glycols, polypropylene glycols, poly(N-vinylpyrrolidone), polymethacrylates, polyphosphazenes, polylactides, polyacrylamides, polyglycolates, polyethylene imines, agaroses, dextrans, gelatins, collagens, polylysines, chitosans, alginates, hyaluronans, pectins and carrageenans that either comprise suitable reactive functionalities or is of formula [Y³—(CH₂)ₛ(CH₂CH₂O)ₙ]ₜQ, wherein —Y³ is a reactive functional group, s is 0, 1 or 2, n is an integer selected from the group ranging from 10 to 1000, -Q is a core group having valency t, and t is an integer selected from the group consisting of 2, 4, 8, 16 and 32.

In certain embodiments, the first polymer comprises a multi-arm polymer. In certain embodiments, the first polymer comprises at least three arms. In certain embodiments, the first polymer comprises at least four arms. In certain embodiments, the first polymer comprises at least five arms. In certain embodiments, the first polymer comprises at least six arms. In certain embodiments, the first polymer comprises at least seven arms. In certain embodiments, the first polymer comprises at least eight arms.

In certain embodiments, the second polymer comprises a multi-arm polymer. In certain embodiments, the second polymer comprises at least three arms. In certain embodiments, the second polymer comprises at least four arms. In certain embodiments, the second polymer comprises at least five arms. In certain embodiments, the second polymer comprises at least six arms. In certain embodiments, the second polymer comprises at least seven arms. In certain embodiments, the second polymer comprises at least eight arms.

In certain embodiments, the first polymer comprises a 2-arm polyethylene glycol polymer. In certain embodiments, the first polymer comprises a 4-arm polyethylene glycol polymer. In certain embodiments, the first polymer comprises an 8-arm polyethylene glycol polymer. In certain embodiments, the first polymer comprises a 16-arm polyethylene glycol polymer. In certain embodiments, the first polymer comprises a 32-arm polyethylene glycol polymer.

In certain embodiments, the second polymer comprises a 2-arm polyethylene glycol polymer. In certain embodiments, the second polymer comprises a 4-arm polyethylene glycol polymer. In certain embodiments, the second polymer comprises an 8-arm polyethylene glycol polymer. In certain embodiments, the second polymer comprises a 16-arm polyethylene glycol polymer. In certain embodiments, the second polymer comprises a 32-arm polyethylene glycol polymer.

In certain embodiments, the first and a second reactive polymer are reacted with said cleavable crosslinker compound, either sequentially or simultaneously.

In certain embodiments, the first and second functional groups are the same.

Only in the context of formulas (PL-1), (PL-2) and (PL-3) the terms used have the following meaning:

The term “a moiety capable of being cleaved by elimination under physiological conditions” refers to a structure comprising a group H—C—(CH═CH)ₘ—C—X′ wherein m is 0 or 1 and X′ is a leaving group, wherein an elimination reaction as described above to remove the elements of HX′ can occur at a rate such that the half-life of the reaction is between 1 and 10,000 hours under physiological conditions of pH and temperature. Preferably, the half-life of the reaction is between 1 and 5,000 hours, and more preferably between 1 and 1,000 hours, under physiological conditions of pH and temperature. By physiological conditions of pH and temperature is meant a pH of between 7 and 8 and a temperature between 30 and 40 degrees centigrade

The term “reactive polymer and reactive oligomer” refers to a polymer or oligomer comprising functional groups that are reactive towards other functional groups, most preferably under mild conditions compatible with the stability requirements of peptides, proteins, and other biomolecules. Suitable functional groups found in reactive polymers include maleimides, thiols or protected thiols, alcohols, acrylates, acrylamides, amines or protected amines, carboxylic acids or protected carboxylic acids, azides, alkynes including cycloalkynes, 1,3-dienes including cyclopentadienes and furans, alpha-halocarbonyls, and N-hydroxysuccinimidyl, N-hydroxysulfosuccinimidyl, or nitrophenyl esters or carbonates.

The term “functional group capable of connecting to a reactive polymer” refers to a functional group that reacts to a corresponding functional group of a reactive polymer to form a covalent bond to the polymer. Suitable functional groups capable of connecting to a reactive polymer include maleimides, thiols or protected thiols, acrylates, acrylamides, amines or protected amines, carboxylic acids or protected carboxylic acids, azides, alkynes including cycloalkynes, 1,3-dienes including cyclopentadienes and furans, alpha-halocarbonyls, and N-hydroxysuccinimidyl, N-hydroxysulfosuccinimidyl, or nitrophenyl esters or carbonates.

The term “substituted” refers to an alkyl, alkenyl, alkynyl, aryl, or heteroaryl group comprising one or more substituent groups in place of one or more hydrogen atoms. Substituent groups may generally be selected from halogen including F, Cl, Br, and I; lower alkyl including linear, branched, and cyclic; lower haloalkyl including fluoroalkyl, chloroalkyl, bromoalkyl, and iodoalkyl; OH; lower alkoxy including linear, branched, and cyclic; SH; lower alkylthio including linear, branched, and cyclic; amino, alkylamino, dialkylamino, silyl including alkylsilyl, alkoxysilyl, and arylsilyl; nitro; cyano; carbonyl; carboxylic acid, carboxylic ester, carboxylic amide; aminocarbonyl; aminoacyl; carbamate; urea; thiocarbamate; thiourea; ketone; sulfone; sulfonamide; aryl including phenyl, naphthyl, and anthracenyl; heteroaryl including 5-member heteroaryls including as pyrrole, imidazole, furan, thiophene, oxazole, thiazole, isoxazole, isothiazole, thiadiazole, triazole, oxadiazole, and tetrazole, 6-member heteroaryls including pyridine, pyrimidine, pyrazine, and fused heteroaryls including benzofuran, benzothiophene, benzoxazole, benzimidazole, indole, benzothiazole, benzisoxazole, and benzisothiazole.

The properties of R¹ and R² may be modulated by the optional addition of electron-donating or electron-withdrawing substituents. The term “electron-donating group” refers to a substituent resulting in a decrease in the acidity of the R¹R²CH; electron-donating groups are typically associated with negative Hammett a or Taft σ* constants and are well-known in the art of physical organic chemistry (Hammett constants refer to aryl/heteroaryl substituents, Taft constants refer to substituents on non-aromatic moieties). Examples of suitable electron-donating substituents include lower alkyl, lower alkoxy, lower alkylthio, amino, alkylamino, dialkylamino, and silyl.

The term “electron-withdrawing group” refers to a substituent resulting in an increase in the acidity of the R¹R²CH group; electron-withdrawing groups are typically associated with positive Hammett a or Taft σ* constants and are well-known in the art of physical organic chemistry. Examples of suitable electron-withdrawing substituents include halogen, difluoromethyl, trifluoromethyl, nitro, cyano, C(═O)—R^{x}, wherein —R^{x} is H, lower alkyl, lower alkoxy, or amino, or S(O)ₘR^{y}, wherein m is 1 or 2 and —R^{y} is lower alkyl, aryl, or heteroaryl. As is well-known in the art, the electronic influence of a substituent group may depend upon the position of the substituent. For example, an alkoxy substituent on the ortho- or para-position of an aryl ring is electron-donating, and is characterized by a negative Hammett a constant, while an alkoxy substituent on the meta-position of an aryl ring is electron-withdrawing and is characterized by a positive Hammett a constant.

The terms “alkyl”, “alkenyl”, and “alkynyl” include linear, branched or cyclic hydrocarbon groups of 1 to 8 carbons or 1 to 6 carbons or 1 to 4 carbons, wherein alkyl is a saturated hydrocarbon, alkenyl includes one or more carbon-carbon double bonds and alkynyl includes one or more carbon-carbon triple bonds. Unless otherwise specified these contain 1 to 6 carbons.

The term “aryl” includes aromatic hydrocarbon groups of 6 to 18 carbons, preferably 6 to 10 carbons, including groups such as phenyl, naphthyl, and anthracenyl. The term “heteroaryl” includes aromatic rings comprising 3 to 15 carbons containing at least one N, O or S atom, preferably 3 to 7 carbons containing at least one N, O or S atom, including groups such as pyrrolyl, pyridyl, pyrimidinyl, imidazolyl, oxazolyl, isoxazolyl, thiazolyl, isothiazolyl, quinolyl, indolyl, indenyl, and similar.

The term “halogen” includes fluoro, chloro, bromo and iodo.

The term “maleimide” is a group of the formula

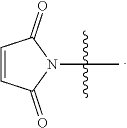 [SMILES: *N1C(=O)C=CC1=O]

In certain embodiments, Z is a hydrogel as disclosed in WO 2020/206358 A1. In particular, in certain embodiments, Z is a hydrogel produced by a method comprising the steps of
(a) providing a first prepolymer comprising a multi-arm polymer —P², wherein said first prepolymer is of formula (PL-4)

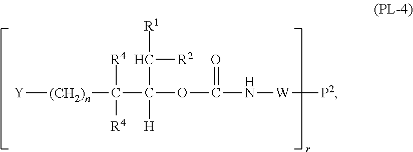

wherein
n is an integer selected from 0, 1, 2, 3, 4, 5 and 6;
r is an integer higher than 2;
—Y is a reactive functional group for connecting said first prepolymer to a second prepolymer;
—R¹ and —R² are independently an electron-withdrawing group, alkyl, or —H, and wherein at least one of —R¹ and —R² is an electron-withdrawing group;
each —R⁴ is independently C₁-C₃ alkyl or the two —R⁴ form together with the carbon atom to which they are attached a 3- to 6-membered ring;
—W— is absent or is

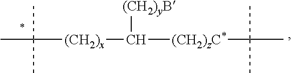

wherein the dashed line marked with the asterisk indicates the attachment to —NH— and the unmarked dashed line indicates the attachment to —P²;
each of x, y, and z is independently an integer selected from 0, 1, 2, 3, 4, 5 and 6;
—B′ is —NH₂, —ONH₂, ketone, aldehyde, —SH, —OH, —CO₂H, carboxamide group, or a group comprising a cyclooctyne or bicyclononyne; and
—C* is carboxamide, thioether, thiosuccinimidyl, triazole, or oxime;
(b) providing the second prepolymer comprising a multi-arm polymer —P¹ wherein each arm is terminated by a reactive functional group —Y″ that reacts with —Y of step (a);
(c) mixing the two prepolymers of steps (a) and (b) under conditions wherein —Y and —Y″ react to form a linkage —Y*-; and optionally
(d) isolating the resulting hydrogel.

Accordingly, —Z is a hydrogel obtainable from the method described above. In certain embodiments, the hydrogel produced by the preceding method is degradable.

In certain embodiments, —Y and —Y″ react under step (c) to form an insoluble hydrogel matrix comprising crosslinks of formula (PL-4′):

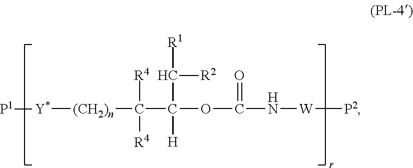

wherein n, r, —P¹, —Y*-, —R⁴, —R¹, —R², —W— and —P² are as defined above.

In certain embodiments, n of formula (PL-4) or (PL-4′) is an integer selected from 1, 2, 3, 4, 5 and 6. In certain embodiments, n of formula (PL-4) or (PL-4′) is an integer selected from 1, 2 and 3. In certain embodiments, n of formula (PL-4) or (PL-4′) is an integer selected from 0, 1, 2 and 3. In certain embodiments, n of formula (PL-4) or (PL-4′) is 1. In certain embodiments, n of formula (PL-4) is 2. In certain embodiments, n of formula (PL-4) or (PL-4′) is 3.

In certain embodiments, the multi-arm —P² of formula (PL-4) or (PL-4′) is an r-armed polymer, wherein r is an integer selected from 2, 3, 4, 5, 6, 7, 8, 9, 10, 11 and 12. In certain embodiments, r of formula (PL-4) or (PL-4′) is an integer selected from 2, 3, 4, 5, 6, 7 and 8. In certain embodiments, r of formula (PL-4) or (PL-4′) is an integer selected from 2, 4, 6 and 8. In certain embodiments, r of formula (PL-4) or (PL-4′) is 2. In certain embodiments, r of formula (PL-4) or (PL-4′) is 4. In certain embodiments, r of formula (PL-4) or (PL-4′) is 6. In certain embodiments, r of formula (PL-4) or (PL-4′) is 8.

In certain embodiments, —P² of formula (PL-4) or (PL-4′) has a molecular weight of at least 1 kDa. In certain embodiments, —P² of formula (PL-4) or (PL-4′) has a molecular weight of 1 to 100 kDa. In certain embodiments, —P² of formula (PL-4) or (PL-4′) has a molecular weight of 1 to 80 kDa. In certain embodiments, —P² of formula (PL-4) or (PL-4′) has a molecular weight of 1 to 60 kDa. In certain embodiments, —P² of formula (PL-4) or (PL-4′) has a molecular weight of 1 to 40 kDa. In certain embodiments, —P² of formula (PL-4) or (PL-4′) has a molecular weight of 1 to 20 kDa. In certain embodiments, —P² of formula (PL-4) or (PL-4′) has a molecular weight of 1 to 10 kDa. In certain embodiments, —P² of formula (PL-4) or (PL-4′) has a molecular weight of 1 to 5 kDa. In certain embodiments, —P² of formula (PL-4) or (PL-4′) has a molecular weight of about 20 kDa. In certain embodiments, —P² of formula (PL-4) or (PL-4′) has a molecular weight of about 40 kDa. In certain embodiments, —P² of formula (PL-4) or (PL-4′) has a molecular weight of about 60 kDa. In certain embodiments, —P² of formula (PL-4) or (PL-4′) has a molecular weight of about 80 kDa.

In certain embodiments, the multi-arm polymer —P¹ of step (b) is an r-armed polymer, wherein r is an integer selected from 2, 3, 4, 5, 6, 7, 8, 9, 10, 11 and 12. In certain embodiments, the multi-arm —P¹ of step (b) is an r-armed polymer, wherein r is an integer selected from 2, 3, 4, 5, 6, 7 and 8. In certain embodiments, the multi-arm —P¹ of step (b) is an r-armed polymer, wherein r is an integer selected from 2, 4, 6 and 8. In certain embodiments, the multi-arm —P¹ of step (b) is an r-armed polymer, wherein r is 2. In certain embodiments, the multi-arm —P¹ of step (b) is an r-armed polymer, wherein r is 4. In certain embodiments, the multi-arm —P¹ of step (b) is an r-armed polymer, wherein r is 6. In certain embodiments, the multi-arm —P¹ of step (b) is an r-armed polymer, wherein r is 8.

In certain embodiments, —P¹ of step (b) has a molecular weight of at least 1 kDa. In certain embodiments, the multi-arm polymer —P¹ of step (b) has a molecular weight of 1 to 100 kDa. In certain embodiments, the multi-arm polymer —P¹ of step (b) has a molecular weight of 1 to 80 kDa. In certain embodiments, the multi-arm polymer —P¹ of step (b) has a molecular weight of 1 to 60 kDa. In certain embodiments, the multi-arm polymer —P¹ of step (b) has a molecular weight of 1 to 40 kDa. In certain embodiments, the multi-arm polymer —P¹ of step (b) has a molecular weight of 1 to 20 kDa. In certain embodiments, the multi-arm polymer —P¹ of step (b) has a molecular weight of 1 to 10 kDa. In certain embodiments, the multi-arm polymer —P¹ of step (b) has a molecular weight of 1 to 5 kDa. In certain embodiments, the multi-arm polymer —P¹ of step (b) has a molecular weight of about 20 kDa. In certain embodiments, the multi-arm polymer —P¹ of step (b) has a molecular weight of about 40 kDa. In certain embodiments, the multi-arm polymer —P¹ of step (b) has a molecular weight of about 60 kDa. In certain embodiments, the multi-arm polymer —P¹ of step (b) has a molecular weight of about 80 kDa.

In certain embodiments, —P¹ of step (b) and —P² of formula (PL-4) or (PL-4′) comprise poly(ethylene glycol) (PEG), poly(ethylene oxide) (PEO), poly(ethylene imine) (PEI), dextrans, hyaluronic acids, or co-polymers thereof. In certain embodiments, —P¹ of step (b) and P² of formula (PL-4) or (PL-4′) are PEG-based polymers. In certain embodiments, —P¹ of step (b) and —P² of formula (PL-4) or (PL-4′) are hyaluronic acid-based polymers.

In certain embodiments, —R¹ and —R² of formula (PL-4) or (PL-4′) are independently electron-withdrawing groups, alkyl, or —H, and wherein at least one of —R¹ and —R² is an electron-withdrawing group.

In certain embodiments, the electron-withdrawing group of —R¹ and —R² of formula (PL-4) or (PL-4′) is —CN, —NO₂, optionally substituted aryl, optionally substituted heteroaryl, optionally substituted alkenyl, optionally substituted alkynyl, —COR³, —SOR³, or —SO₂R³, wherein —R³ is —H, optionally substituted alkyl, optionally substituted aryl, optionally substituted arylalkyl, optionally substituted heteroaryl, optionally substituted heteroarylalkyl, —OR⁸ or —NR⁸₂, wherein each —R⁸ is independently —H or optionally substituted alkyl, or both —R⁸ groups are taken together with the nitrogen to which they are attached to form a heterocyclic ring; or —SR⁹, wherein —R⁹ is optionally substituted alkyl, optionally substituted aryl, optionally substituted arylalkyl, optionally substituted heteroaryl, or optionally substituted heteroarylalkyl.

In certain embodiments, the electron-withdrawing group of —R¹ and —R² of formula (PL-4) or (PL-4′) is —CN. In certain embodiments, the electron-withdrawing group of —R¹ and —R² of formula (PL-4) or (PL-4′) is —NO₂. In certain embodiments, the electron-withdrawing group of —R¹ and —R² of formula (PL-4) or (PL-4′) is optionally substituted aryl containing 6 to 10 carbons. In certain embodiments, the electron-withdrawing group of —R¹ and —R² of formula (PL-4) or (PL-4′) is optionally substituted phenyl, naphthyl, or anthracenyl. In certain embodiments, the electron-withdrawing group of —R¹ and —R² of formula (PL-4) or (PL-4′) is optionally substituted heteroaryl comprising 3 to 7 carbons and containing at least one N, O, or S atom. In certain embodiments, the electron-withdrawing group of —R¹ and —R² of formula (PL-4) or (PL-4′) is optionally substituted pyrrolyl, pyridyl, pyrimidinyl, imidazolyl, oxazolyl, isoxazolyl, thiazolyl, isothiazolyl, quinolyl, indolyl, or indenyl. In certain embodiments, the electron-withdrawing group of —R¹ and —R² of formula (PL-4) or (PL-4′) is optionally substituted alkenyl containing 2 to 20 carbon atoms. In certain embodiments, the electron-withdrawing group of —R¹ and —R² of formula (PL-4) or (PL-4′) is optionally substituted alkynyl containing 2 to 20 carbon atoms. In certain embodiments, the electron-withdrawing group of —R¹ and —R² of formula (PL-4) or (PL-4′) is —COR³, —SOR³, or —SO₂R³, wherein R³ is —H, optionally substituted alkyl containing 1 to 20 carbon atoms, optionally substituted aryl, optionally substituted arylalkyl, optionally substituted heteroaryl, optionally substituted heteroarylalkyl, —OR⁸ or —NR⁸₂, wherein each —R⁸ is independently —H or optionally substituted alkyl containing 1 to 20 carbon atoms, or both —R⁸ groups are taken together with the nitrogen to which they are attached to form a heterocyclic ring. In certain embodiments, the electron-withdrawing group of —R¹ and —R² of formula (PL-4) or (PL-4′) is —SR⁹, wherein —R⁹ is optionally substituted alkyl containing 1 to 20 carbon atoms, optionally substituted aryl, optionally substituted arylalkyl, optionally substituted heteroaryl, or optionally substituted heteroarylalkyl. In certain embodiments, at least one of —R¹ and —R² is —CN or —SO₂R³.

In certain embodiments, at least one of —R¹ and —R² of formula (PL-4) or (PL-4′) is —CN, —SOR³ or —SO₂R³. In certain embodiments, at least one of —R¹ and —R² of formula (PL-4) or (PL-4′) is —CN or —SO₂R³. In certain embodiments, at least one of —R¹ and —R² of formula (PL-4) or (PL-4′) is —CN or —SO₂R³, wherein —R³ is optionally substituted alkyl, optionally substituted aryl, or —NR⁸₂. In certain embodiments, at least one of —R¹ and —R² of formula (PL-4) or (PL-4′) is —CN, —SO₂N(CH₃)₂, —SO₂CH₃, phenyl substituted with —SO₂, phenyl substituted with —SO₂ and —Cl, —SO₂N(CH₂CH₂)₂O, —SO₂CH(CH₃)₂, —SO₂N(CH₃)(CH₂CH₃), or —SO₂N(CH₂CH₂OCH₃)₂.

In certain embodiments, each —R⁴ of formula (PL-4) or (PL-4′) is independently C₁-C₃ alkyl or taken together may form a 3- to 6-membered ring. In certain embodiments, each —R⁴ of formula (PL-4) or (PL-4′) is independently C₁-C₃ alkyl. In certain embodiments, both —R⁴ of formula (PL-4) or (PL-4′) are methyl.

In certain embodiments, —Y and —Y″ are independently selected from the group consisting of amine, aminooxy, ketone, aldehyde, maleimidyl, thiol, alcohol, azide, 1,2,4,6-tetrazinyl, trans-cyclooctenyl, bicyclononynyl, cyclooctynyl, and protected variants thereof.

In certain embodiments, Y and Y″ may react with each other such as in a selective way. For example, when —Y is amine, —Y″ is carboxylic acid, active ester, or active carbonate to yield a residual connecting functional group —Y*- that is amide or carbamate. As another example, when —Y is azide, —Y″ is alkynyl, bicyclononynyl, or cyclooctynyl to yield a residual connecting functional group —Y*- that is 1,2,3-triazole. As another example, when —Y is NH₂O, —Y″ is ketone or aldehyde to yield a residual connecting functional group —Y*- that is oxime. As another example, when —Y is SH, —Y″ is maleimide or halocarbonyl to yield a residual connecting functional group —Y*- that is thiosuccinimidyl or thioether. Similarly, these roles of —Y and —Y″ can be reversed to yield —Y*- of opposing orientation.

In certain embodiments, —Y*- comprises an amide, oxime, 1,2,3-triazole, thioether, thiosuccinimide, or ether. In certain embodiments, —Y*- is -L²-.

These conjugation reactions may be performed under conditions known in the art, for example when —Y is azide and —Y″ is cyclooctyne the conjugation occurs in any solvent wherein both components show adequate solubility, although it is known that aqueous solutions show more favorable reaction rates. When mixed in an appropriate solvent, typically an aqueous buffer at a pH of 2 to 7 when —Y and —Y″ are azide/cyclooctyne, or at a pH of 6 to 9 when —Y and —Y″ are an activated ester and an amine, the —Y and —Y″ groups react to form an insoluble hydrogel matrix comprising crosslinks of formula (PL-4′). This process may be carried out in bulk phase, or under conditions of emulsification in a mixed organic/aqueous system so as to form microparticle suspensions such as microspheres that are suitable for injection.

Only in the context of formulas (PL-4) and (PL-4′) the terms used have the following meaning:

The term “alkyl” refers to linear, branched, or cyclic saturated hydrocarbon groups of 1 to 20, 1 to 12, 1 to 8, 1 to 6, or 1 to 4 carbon atoms. In certain embodiments an alkyl is linear or branched. Examples of linear or branched alkyl groups include methyl, ethyl, n-propyl, isopropyl, n-butyl, t-butyl, isobutyl, sec-butyl, n-pentyl, n-hexyl, n-heptyl, n-octyl, n-nonyl, and n-decyl. In certain embodiments an alkyl is cyclic. Examples of cyclic alkyl groups include cyclopropyl, cyclobutyl, cyclopentyl, cyclopentadienyl, and cyclohexyl.

The term “alkoxy” refers to alkyl groups bonded to oxygen, including methoxy, ethoxy, isopropoxy, cyclopropoxy, and cyclobutoxy.

The term “alkenyl” refers to non-aromatic unsaturated hydrocarbons with carbon-carbon double bonds and 2 to 20, 2 to 12, 2 to 8, 2 to 6, or 2 to 4 carbon atoms.

The term “alkynyl” refers to non-aromatic unsaturated hydrocarbons with carbon-carbon triple bonds and 2 to 20, 2 to 12, 2 to 8, 2 to 6, or 2 to 4 carbon atoms.

The term “aryl” refers to aromatic hydrocarbon groups of 6 to 18 carbons, preferably 6 to 10 carbons, including groups such as phenyl, naphthyl, and anthracenyl. The term “heteroaryl” refers to aromatic rings comprising 3 to 15 carbons comprising at least one N, O or S atom, preferably 3 to 7 carbons comprising at least one N, O or S atom, including groups such as pyrrolyl, pyridyl, pyrimidinyl, imidazolyl, oxazolyl, isoxazolyl, thiazolyl, isothiazolyl, quinolyl, indolyl, and indenyl.

In certain embodiments, alkenyl, alkynyl, aryl or heteroaryl moieties may be coupled to the remainder of the molecule through an alkyl linkage. Under those circumstances, the substituent will be referred to as alkenylalkyl, alkynylalkyl, arylalkyl or heteroarylalkyl, indicating that an alkylene moiety is between the alkenyl, alkynyl, aryl or heteroaryl moiety and the molecule to which the alkenyl, alkynyl, aryl or heteroaryl is coupled.

The term “halogen” or “halo” refers to bromo, fluoro, chloro or iodo.

The term “heterocyclic ring” or “heterocyclyl” refers to a 3- to 15-membered aromatic or non-aromatic ring comprising at least one N, O, or S atom. Examples include piperidinyl, piperazinyl, tetrahydropyranyl, pyrrolidine, and tetrahydrofuranyl, as well as the exemplary groups provided for the term “heteroaryl” above. In certain embodiments, a heterocyclic ring or heterocyclyl is non-aromatic. In certain embodiments, a heterocyclic ring or heterocyclyl is aromatic.

The term “optionally substituted” refers to a group that may be unsubstituted or substituted by one or more (e.g., 1, 2, 3, 4 or 5) of the substituents which may be the same or different.

Examples of substituents include alkyl, alkenyl, alkynyl, halogen, —CN, —OR^{aa}, —SR^{aa}, —NR^{aa}R^{bb}, —NO₂, —C═NH(OR^{aa}), —C(O)R^{aa}, —OC(O)R^{aa}, —C(O)OR^{aa}, —C(O)NR^{aa}R^{bb}, —OC(O)NR^{aa}R^{bb}, —NR^{aa}C(O)R^{bb}, —NR—C(O)OR^{bb}, —S(O)R^{aa}, —S(O)₂R^{aa}, —NR^{aa}S(O)R^{bb}, —C(O)NR^{aa}S(O)R^{bb}, —NR^{aa}S(O)₂R^{bb}, —C(O)NR^{aa}S(O)₂R^{bb}, —S(O)NR^{aa}R^{bb}, —S(O)₂NR^{aa}R^{bb}, —P(O)(OR^{aa})(OR^{bb}), heterocyclyl, heteroaryl, or aryl, wherein the alkyl, alkenyl, alkynyl, cycloalkyl, heterocyclyl, heteroaryl, and aryl are each independently optionally substituted by —R^{cc}, wherein —R^{aa} and —R^{bb} are each independently —H, alkyl, alkenyl, alkynyl, heterocyclyl, heteroaryl, or aryl, or —R^{aa} and —R^{bb} are taken together with the nitrogen atom to which they attach to form a heterocyclyl, which is optionally substituted by alkyl, alkenyl, alkynyl, halogen, hydroxyl, alkoxy, or —CN, and wherein: each —R^{cc} is independently alkyl, alkenyl, alkynyl, halogen, heterocyclyl, heteroaryl, aryl, —CN, or —NO₂.

In certain embodiments, Z is a polymer network formed through the physical aggregation of polymer chains, which physical aggregation is preferably caused by hydrogen bonds, crystallization, helix formation or complexation. In certain embodiments, such polymer network is a thermogelling polymer.

In certain embodiments, Z comprises a moiety selected from the group consisting of:

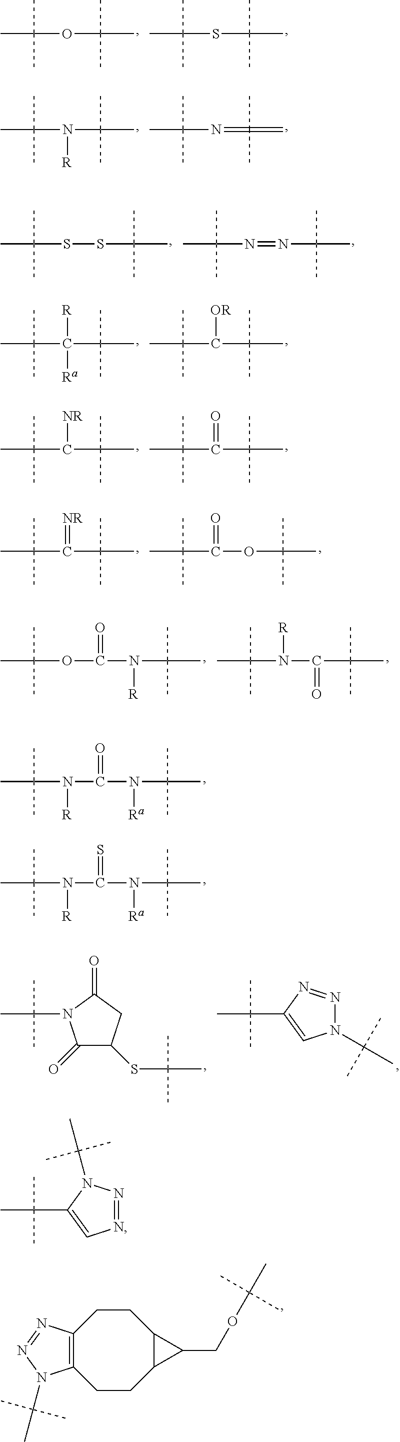

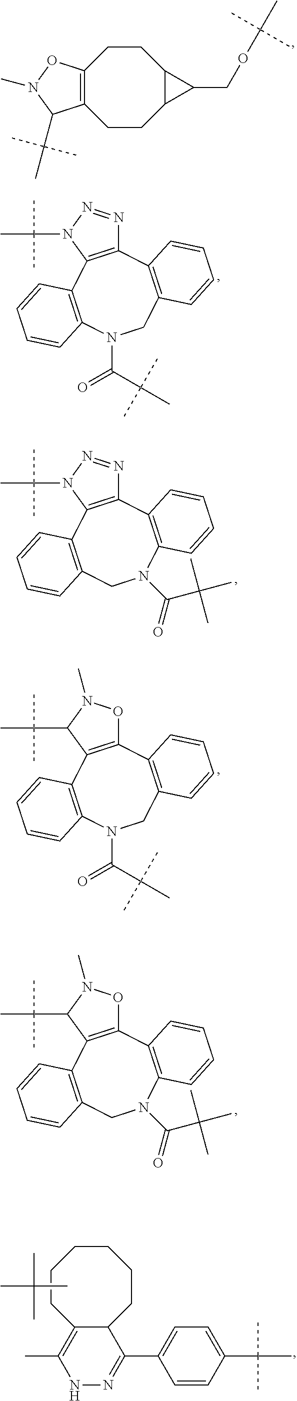

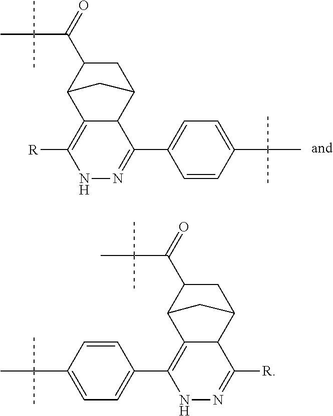

In certain embodiments, the conjugate of the present invention or the pharmaceutically acceptable salt thereof is of formula (Ia), (Ib), (Ic) or (Id):

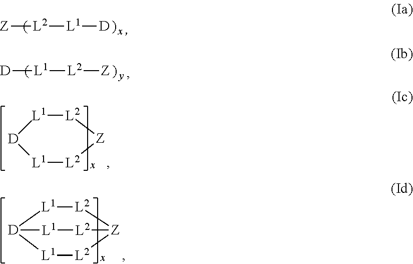

wherein
each -D, -L²- and Z are defined as above and each -L¹- is independently of formula (I);
x is an integer of at least 1; and
y is an integer selected from the group consisting of 2, 3, 4 and 5.

It is understood that even though one -D can be conjugated to multiple -L¹- moieties, the corresponding drug moiety is for simplicity represented by “-D” and the drug by “D-H”.

In certain embodiments, the conjugate is of formula (Ia), (Ic) or (Id) and Z is a hydrogel. In such cases, Z comprises a plurality of moieties -L²-L¹-D and it is understood that no upper limit for x can be provided.

In certain embodiments, the conjugate is of formula (Ia). In certain embodiments, the conjugate is of formula (Ib). In certain embodiments, the conjugate is of formula (Ic). In certain embodiments, the conjugate is of formula (Id). In certain embodiments, the conjugate is of formula (Ia) with x=1.

In certain embodiments, the conjugate is of formula (Ia) and Z is a hydrogel.

In certain embodiments, the conjugate is of formula (Ia), (Ic) or (Id), Z is a water-soluble polymeric moiety and x ranges from 2 to 1000, such as from 2 to 1500, such as from 2 to 1000, such as from 2 to 500, such as from 2 to 250 or such as from 2 to 100. In certain embodiments, the conjugate is of formula (Ia), (Ic) or (Id), Z is a water-soluble polymeric moiety and x is 20.

In certain embodiments, the conjugate is of formula (Ia), (Ic) or (Id), Z is a water-soluble polymeric moiety and x is 19. In certain embodiments, the conjugate is of formula (Ia), (Ic) or (Id), Z is a water-soluble polymeric moiety and x is 18. In certain embodiments, the conjugate is of formula (Ia), (Ic) or (Id), Z is a water-soluble polymeric moiety and x is 17. In certain embodiments, the conjugate is of formula (Ia), (Ic) or (Id), Z is a water-soluble polymeric moiety and x is 16. In certain embodiments, the conjugate is of formula (Ia), (Ic) or (Id), Z is a water-soluble polymeric moiety and x is 15. In certain embodiments, the conjugate is of formula (Ia), (Ic) or (Id), Z is a water-soluble polymeric moiety and x is 14. In certain embodiments, the conjugate is of formula (Ia), (Ic) or (Id), Z is a water-soluble polymeric moiety and x is 13. In certain embodiments, the conjugate is of formula (Ia), (Ic) or (Id), Z is a water-soluble polymeric moiety and x is 12. In certain embodiments, the conjugate is of formula (Ia), (Ic) or (Id), Z is a water-soluble polymeric moiety and x is 11. In certain embodiments, the conjugate is of formula (Ia), (Ic) or (Id), Z is a water-soluble polymeric moiety and x is 10. In certain embodiments, the conjugate is of formula (Ia), (Ic) or (Id), Z is a water-soluble polymeric moiety and x is 9. In certain embodiments, the conjugate is of formula (Ia), (Ic) or (Id), Z is a water-soluble polymeric moiety and x is 8. In certain embodiments, the conjugate is of formula (Ia), (Ic) or (Id), Z is a water-soluble polymeric moiety and x is 7. In certain embodiments, the conjugate is of formula (Ia), (Ic) or (Id), Z is a water-soluble polymeric moiety and x is 6. In certain embodiments, the conjugate is of formula (Ia), (Ic) or (Id), Z is a water-soluble polymeric moiety and x is 5. In certain embodiments, the conjugate is of formula (Ia), (Ic) or (Id), Z is a water-soluble polymeric moiety and x is 4. In certain embodiments, the conjugate is of formula (Ia), (Ic) or (Id), Z is a water-soluble polymeric moiety and x is 3. In certain embodiments, the conjugate is of formula (Ia), (Ic) or (Id), Z is a water-soluble polymeric moiety and x is 2.

In certain embodiments, the conjugate is of formula (Ib), Z is a water-soluble polymeric moiety and y is 1. In certain embodiments, the conjugate is of formula (Ib), Z is a water-soluble polymeric moiety and y is 2. In certain embodiments, the conjugate is of formula (Ib), Z is a water-soluble polymeric moiety and y is 3. In certain embodiments, the conjugate is of formula (Ib), Z is a water-soluble polymeric moiety and y is 4. In certain embodiments, the conjugate is of formula (Ib), Z is a water-soluble polymeric moiety and y is 5.

The conjugates of the present invention release one or more types of drug over an extended period of time, i.e. they are sustained-release conjugates. In certain embodiments, the release occurs with a release half-life ranging between 1 day and 4 months. In certain embodiments, the release occurs with a release half-life ranging between 2 days and 2 months. In certain embodiments, the release occurs with a release half-life between 4 days and 2 months. In certain embodiments, the release half-life may also range from 6 days to 1 month, 7 days to 40 days, 4 days to 15 days or 3 days to 7 days.

In certain embodiments, steps (d) and (e) of the method for synthesizing a conjugate of the present invention are not optional.

In certain embodiments, conjugation of at least one Z moiety is not optional.

In certain embodiments, at least one Z moiety is conjugated to at least one intermediate (A) in between steps (b) and (c). In certain embodiments, at least one Z moiety is conjugated to at least one intermediate (A) in between steps (c) and (d). In certain embodiments, at least one Z moiety is conjugated to at least one intermediate (A) in between steps (c) and (f). In certain embodiments, steps (d) and (e) are not optional and at least one Z moiety is conjugated to at least one intermediate (B) in between steps (d) and (e). In certain embodiments, step (e) is not optional and at least one Z moiety is conjugated to at least one intermediate (B) in between steps (e) and (f).

In certain embodiments, at least one Z moiety is conjugated during step (b). In certain embodiments, at least one Z moiety is conjugated during step (c). In certain embodiments, step (d) is not optional and at least one Z moiety is conjugated during step (d). In certain embodiments, step (e) is not optional and at least one Z moiety is conjugated during step (e).

In certain embodiments, one Z moiety is conjugated during step (b). In certain embodiments, one Z moiety is conjugated during step (c). In certain embodiments, step (d) is not optional and one Z moiety is conjugated during step (d). In certain embodiments, step (e) is not optional and one Z moiety is conjugated during step (e).

In certain embodiments, intermediate (A) is isolated before step (c). In certain embodiments, step (d) is not optional and intermediate (B) is isolated before step (d). In certain embodiments, steps (d) and (e) are not optional and intermediate (B) is isolated before step (e).

In certain embodiments, the conjugate or intermediate resulting from steps (c), (d) or (e) is purified by ion exchange chromatography.

In certain embodiments, in step (a) of the method the reagent comprises a linker -L*- of formula (II), wherein -L*- is substituted with one moiety -L²-Y and step (b) results in the formation of an intermediate (A) which is isolated before being subjected to deprotection conditions and Z.

In certain embodiments, in step (a) of the method the reagent comprises a linker -L*- of formula (II), wherein -L*- is substituted with one moiety -L²-Y and step (b) results in the formation of an intermediate (A) which is conjugated to Z.

In certain embodiments, the primary or secondary amine-comprising drug of step (b) of the method is a peptide or protein. In certain embodiments, the primary or secondary amine-comprising drug of step (b) of the method is a protein.

In certain embodiments, step (d) of the method of the present invention is not optional. In certain embodiments, step (e) of the method of the present invention is not optional.

In certain embodiments, the deprotection conditions refer to a solution comprising a buffering agent.

In certain embodiments, the shift conditions refer to a solution comprising a buffering agent.

Exemplary buffering agents may be selected from the group consisting of histidine, 1,3-diaminopropane, 2-(N-morpholino)ethanesulfonic acid (MES), 2-bis(2-hydroxyethyl)amino-2-(hydroxymethyl)-1,3-propanediol (BIS-TRIS), acetic acid, adipic acid, ammonia, arginine, boric acid, carbonic acid, citric acid, diethanolamine, ethanolamine, ethylenediamine, formic acid, gluconic acid, glutaric acid, glycine, glycinamide, guanidine, histamine, imidazole, lysine, malic acid, N-(2-hydroxy-1,1-bis(hydroxymethyl)ethyl)glycine (TRICINE), N-(2-hydroxyethyl)piperazine-N′-(2-ethanesulfonic acid) (HEPES), N-[tris(hydroxymethyl)methyl]-2-aminoethanesulfonic acid (TES), phosphoric acid, piperazine, propionic acid, pyruvic acid, spermidine, spermine, succinic acid, tartronic acid, triethanolamine (TEA), tromethamine (TRIS), tyrosine and mixtures thereof.

In certain embodiments, the deprotection conditions refer to a solution comprising a buffering agent of a pH not higher than 10. In certain embodiments, the deprotection conditions refer to a solution comprising a buffering agent of a pH from about 3 to about 9. In certain embodiments, the deprotection conditions refer to a solution comprising a buffering agent of a pH from about 4 to about 8. In certain embodiments, the deprotection conditions refer to a solution comprising a buffering agent of a pH from about 5 to about 7. In certain embodiments, the deprotection conditions refer to a solution comprising a buffering agent of a pH of about 6. In certain embodiments, the deprotection conditions refer to a solution comprising a buffering agent of a pH of about 7. In certain embodiments, the deprotection conditions refer to a solution comprising a buffering agent of a pH of about 7.4. In certain embodiments, the deprotection conditions refer to a solution comprising a buffering agent of a pH of 7.4.

In certain embodiments, the shift conditions refer to a solution comprising a buffering agent of a pH from about 5 to about 9. In certain embodiments, the shift conditions refer to a solution comprising a buffering agent of a pH from about 6 to about 8. In certain embodiments, the shift conditions refer to a solution comprising a buffering agent of a pH of about 7. In certain embodiments, the shift conditions refer to a solution comprising a buffering agent of a pH of about 7.4. In certain embodiments, the shift conditions refer to a solution comprising a buffering agent of a pH of 7.4.

In certain embodiments, the deprotection conditions refer a solution comprising a buffering agent and a scavenger.

Exemplary scavengers may be selected from the group consisting of ammonium phosphate, acetyllysine, m-cresol, dithiothreitol, 1,2 ethanedithiol, hydrazine, hydroxylamine, imidazole, 2-mercapto pyridine, 4-mercapto pyridine, 2-methoxyphenol, 4-methoxyphenol, morpholine, phenol, piperazine, proline, thioaniline, thioanisole, N,N,N′-trimethylethylene diamine, triethylsilane, triisopropylsilane and tris(hydroxymethyl)methanamine.

In certain embodiments, the deprotection conditions refer to a solution comprising a buffering agent and N,N,N′-trimethylethylene diamine.

In certain embodiments, the deprotection conditions refer to a solution comprising an organic solvent, such as a polar protic solvent or a polar aprotic solvent.

In certain embodiments, the deprotection conditions refer to a solution comprising an organic solvent and an acid.

In certain embodiments, the acid is trifluoroacetic acid. In certain embodiments, the acid is hydrochloric acid.

In certain embodiments, the deprotection conditions refer to a solution comprising an organic solvent and a base.

In certain embodiments, the base is 1,8-diazabicyclo[5.4.0]undec-7-ene (DBU). In certain embodiments, the base is piperidine.

In certain embodiments, the deprotection conditions refer to a solution comprising a polar aprotic solvent.

Exemplary apolar protic solvents may be selected from the group consisting of dimethyl sulfoxide, 1,2-dimethoxyether, 1,3-dimethyl-2-imidazolidinone, 1,3-dioxolane, 1,4-dioxane, 2,5-dimethyltetrahydrofuran, 2-methyltetrahydrofuran, 4-acetyl morpholine, 4-propionyl morpholine, acetone, acetonitrile, diethyl carbonate, diethyl ether, dimethyl carbonate, ethyl acetate, ethyl formate, ethyl lactate, ethylene carbonate, gamma-butyrolactone, gamma-valerolactone, hexamethylphosphoramide, methyl acetate, methyl carbonate, monomethyl ether acetate, N,N′-dimethylpropyleneurea, N,N-dimethylacetamide, N,N-dimethylformamide, N-formyl morpholine, N-methyl-2-pyrrolidone, propylene carbonate, sulfolane, tetrahydrofuran, tetrahydropyran, tripyrrolidinophosphoric acid triamide and mixtures thereof.

In certain embodiments, the deprotection conditions refer to a solution comprising a polar protic solvent.

Exemplary polar protic solvents may be selected from the group consisting of ethanol, 1,4-butanediol, acetic acid, cyclohexanol, diethylene glycol, diethylene glycol monoethyl ether, diethylene glycol monomethyl ether, ethylene diamine, ethylene glycol, ethylene glycol monoethyl ether, ethylene glycol monomethyl ether, formamide, formic acid, glycerine, isobutanol, isopropanol, methanesulfonic acid, methanol, n-butanol, n-hexanol, n-pentanol, n-propanol, propionic acid, propylene diamine, propylene glycol, propylene glycol monoethyl ether, propylene glycol monomethyl ether, sec-butanol, t-butanol, triethylene glycol monoethyl ether, triethylene glycol monomethyl ether, triethylene glycol, trifluoroacetic acid, water and mixtures thereof.

In certain embodiments, the deprotection conditions also provide for shift conditions.

Another aspect of the present invention is a pharmaceutical composition comprising at least one conjugate of the present invention or a pharmaceutical salt thereof.

In certain embodiments, the pharmaceutical composition comprises at least one conjugate of the present invention or a pharmaceutical salt thereof, such as one conjugate. In certain embodiments, the pharmaceutical composition comprises two conjugates of the present invention. In certain embodiments, the pharmaceutical composition comprises three conjugates of the present invention.

Such pharmaceutical composition may have a pH ranging from pH 3 to pH 8, such as ranging from pH 4 to pH 6 or ranging from pH 4 to pH 5. In certain embodiments, the pH of the pharmaceutical composition is about 4. In certain embodiments, the pH of the pharmaceutical composition is about 4.5. In certain embodiments, the pH of the pharmaceutical composition is about 5. In certain embodiments, the pH of the pharmaceutical composition is about 5.5.

In certain embodiments, the pH of the pharmaceutical composition is 4. In certain embodiments, the pH of the pharmaceutical composition is 4.5. In certain embodiments, the pH of the pharmaceutical composition is 5. In certain embodiments, the pH of the pharmaceutical composition is 5.5.

In certain embodiments, such pharmaceutical composition is a suspension formulation.

In certain embodiments such pharmaceutical is a dry composition. It is understood that such dry composition may be obtained by drying, such as lyophilizing, a suspension composition.

If the pharmaceutical composition is a parenteral composition, suitable excipients may be categorized as, for example, buffering agents, isotonicity modifiers, preservatives, stabilizers, anti-adsorption agents, oxidation protection agents, viscosifiers/viscosity enhancing agents, anti-agglomeration agents or other auxiliary agents. However, in some cases, one excipient may have dual or triple functions. Excipient may be selected from the group consisting of

(i) Buffering agents: physiologically tolerated buffers to maintain pH in a desired range, such as sodium phosphate, bicarbonate, succinate, histidine, citrate and acetate, sulfate, nitrate, chloride, pyruvate; antacids such as Mg(OH)₂ or ZnCO₃ may be also used;

(ii) Isotonicity modifiers: to minimize pain that can result from cell damage due to osmotic pressure differences at the injection depot; glycerin and sodium chloride are examples; effective concentrations can be determined by osmometry using an assumed osmolality of 285-315 mOsmol/kg for serum;

(iii) Preservatives and/or antimicrobials: multidose parenteral formulations require the addition of preservatives at a sufficient concentration to minimize risk of patients becoming infected upon injection and corresponding regulatory requirements have been established;

typical preservatives include m-cresol, phenol, methylparaben, ethylparaben, propylparaben, butylparaben, chlorobutanol, benzyl alcohol, phenylmercuric nitrate, thimerosol, sorbic acid, potassium sorbate, benzoic acid, chlorocresol and benzalkonium chloride;

(iv) Stabilizers: Stabilisation is achieved by strengthening of the protein-stabilising forces, by destabilisation of the denatured state, or by direct binding of excipients to the protein; stabilizers may be amino acids such as alanine, arginine, aspartic acid, glycine, histidine, lysine, proline, sugars such as glucose, sucrose, trehalose, polyols such as glycerol, mannitol, sorbitol, salts such as potassium phosphate, sodium sulphate, chelating agents such as EDTA, hexaphosphate, ligands such as divalent metal ions (zinc, calcium, etc.), other salts or organic molecules such as phenolic derivatives; in addition, oligomers or polymers such as cyclodextrins, dextran, dendrimers, PEG or PVP or protamine or HSA may be used;

(v) Anti-adsorption agents: mainly ionic or non-ionic surfactants or other proteins or soluble polymers are used to coat or adsorb competitively to the inner surface of the formulation's container; e.g., poloxamer (Pluronic F-68), PEG dodecyl ether (Brij 35), polysorbate 20 and 80, dextran, polyethylene glycol, PEG-polyhistidine, BSA and HSA and gelatins; chosen concentration and type of excipient depends on the effect to be avoided but typically a monolayer of surfactant is formed at the interface just above the CMC value;

(vi) Oxidation protection agents: antioxidants such as ascorbic acid, ectoine, methionine, glutathione, monothioglycerol, morin, polyethylenimine (PEI), propyl gallate, and vitamin E; chelating agents such as citric acid, EDTA, hexaphosphate, and thioglycolic acid may also be used;

(vii) Viscosifiers or viscosity enhancers: retard settling of the particles in the vial and syringe and are used in order to facilitate mixing and resuspension of the particles and to make the suspension easier to inject (i.e., low force on the syringe plunger); suitable viscosifiers or viscosity enhancers are, for example, carbomer viscosifiers like Carbopol 940, Carbopol Ultrez 10, cellulose derivatives like hydroxypropylmethylcellulose (hypromellose, HPMC) or diethylaminoethyl cellulose (DEAE or DEAE-C), colloidal magnesium silicate (Veegum) or sodium silicate, hydroxyapatite gel, tricalcium phosphate gel, xanthans, carrageenans like Satia gum UTC 30, aliphatic poly(hydroxy acids), such as poly(D,L- or L-lactic acid) (PLA) and poly(glycolic acid) (PGA) and their copolymers (PLGA), terpolymers of D,L-lactide, glycolide and caprolactone, poloxamers, hydrophilic poly(oxyethylene) blocks and hydrophobic poly(oxypropylene) blocks to make up a triblock of poly(oxyethylene)-poly(oxypropylene)-poly(oxyethylene) (e.g. Pluronic©), polyetherester copolymer, such as a polyethylene glycol terephthalate/polybutylene terephthalate copolymer, sucrose acetate isobutyrate (SAIB), dextran or derivatives thereof, combinations of dextrans and PEG, polydimethylsiloxane, collagen, chitosan, polyvinyl alcohol (PVA) and derivatives, polyalkylimides, poly (acrylamide-co-diallyldimethyl ammonium (DADMA)), polyvinylpyrrolidone (PVP), glycosaminoglycans (GAGs) such as dermatan sulfate, chondroitin sulfate, keratan sulfate, heparin, heparan sulfate, hyaluronan, ABA triblock or AB block copolymers composed of hydrophobic A-blocks, such as polylactide (PLA) or poly(lactide-co-glycolide) (PLGA), and hydrophilic B-blocks, such as polyethylene glycol (PEG) or polyvinyl pyrrolidone; such block copolymers as well as the abovementioned poloxamers may exhibit reverse thermal gelation behavior (fluid state at room temperature to facilitate administration and gel state above sol-gel transition temperature at body temperature after injection);

(viii) Spreading or diffusing agent: modifies the permeability of connective tissue through the hydrolysis of components of the extracellular matrix in the interstitial space such to hyaluronic acid, a polysaccharide found in the intercellular space of connective tissue; a spreading agent such as hyaluronidase temporarily decreases the viscosity of the extracellular matrix and promotes diffusion of injected drugs;

(ix) Anti-agglomeration agents, such as propylene glycol; and

(x) Other auxiliary agents: such as wetting agents, viscosity modifiers, antibiotics, hyaluronidase; acids and bases such as hydrochloric acid and sodium hydroxide are auxiliary agents necessary for pH adjustment during manufacture.

In another aspect, the present invention relates to a conjugate of the present invention or a pharmaceutical composition comprising a conjugate of the present invention for use as a medicament.

In another aspect, the present invention relates to a conjugate or a pharmaceutically acceptable salt thereof of the present invention or a pharmaceutical composition comprising a conjugate of the present invention for use in a method of treating a disease that can be treated with D-H or its pharmaceutically acceptable salt thereof.

In a further aspect, the present invention relates to a method of preventing a disease or treating a patient suffering from a disease that can be prevented or treated with D-H comprising administering an effective amount of the conjugate or its pharmaceutically acceptable salt thereof of the present invention or the pharmaceutical compositions comprising said conjugates to the patient.

As the present invention is applicable to all drug molecules comprising a primary or secondary amine, it is impossible to further specify the disease that can be treated. However, it is evident to the person skilled in the art which disease can be treated with a particular conjugate.

### EXAMPLES

Materials and Methods

Chemicals

All materials were commercially available except where stated otherwise. Monoclonal antibody CTLA-4 mAB (AMO-M6104, CAS No. 477202-00-9) was obtained from AbMole Bioscience Inc., Houston, Tex., US. HHC^{MET} (EVQLVESGGGLVQAGGSLRLSCAASGGTFSFYGMGWFRQAPGKEQEFVA DIRTSAGRTYYADSVKGRFTISRDNAKNTVYLQMNSLKPEDTAVYYCAAEMSGISG WDYWGQGTQVTVSSGGGGSGGGSEVQLVESGGGLVQAGGSLRLSCAASGGTFSFY GMGWFRQAPGKEQEFVADIRTSAGRTYYADSVKGRFTISRDNAKNTVYLQMNSLKP EDTAVYYCAAEMSGISGWDYWGQGTQVTVSS; SEQ ID NO:1) was custom made and sourced from an external supplier where expression of the protein was performed from E. coli followed by standard purification strategies known to the one skilled in the art.

Reactions Reactions were performed with anhydrous solvents (CH₂Cl₂, DMSO, DMF, THF, acetonitrile) purchased from Sigma-Aldrich Chemie GmbH, Munich, Germany. Generally, reactions were stirred at room temperature and monitored by LCMS.

RP-HPLC Purification

Preparative RP-HPLC purifications were performed with a Waters 600 controller with a 2487 Dual Absorbance Detector or an Agilent Infinity 1260 preparative system using a Waters XBridge BEH300 Prep C18 10 μm, 150×30 mm column as stationary phase. Products were detected at 215 nm. Linear gradients of solvent system A (water containing 0.1% TFA v/v) and solvent system B (acetonitrile containing 0.1% TFA v/v) were used.

HPLC fractions containing product were pooled and lyophilized if not stated otherwise.

Flash Chromatography

Flash chromatography purifications were performed on an Isolera One system from Biotage AB, Sweden, using Biotage KP-Sil silica cartridges. Products were detected at 254 nm or 280 nm.

RP-LPLC Purification

Low pressure RP chromatography purifications were performed on an Isolera One system from Biotage AB, Sweden, using Biotage SNAP C18 cartridges. Products were detected at 215 nm and 280 nm. Linear gradients of solvent system A (water containing 0.1% TFA v/v) and solvent system B (acetonitrile containing 0.1% TFA v/v). Fractions containing product were pooled and lyophilized if not stated otherwise.

UPLC-MS Analysis

Analytical ultra-performance LC (UPLC)-MS was performed on a Waters Acquity system or an Agilent 1290 Infinity II equipped with a Waters BEH300 C18 column (2.1×50 mm, 1.7 μm particle size or 2.1×100 mm, 1.7 μm particle size); solvent A: water containing 0.05% TFA (v/v), solvent B: acetonitrile containing 0.04% TFA (v/v) coupled to a Waters Micromass ZQ or coupled to an Agilent Single Quad MS system.

OPA Assay

Amine content of the amine-HA was determined by reacting the free amino groups with o-phthalaldehyde (OPA) and N-acetylcysteine under alkaline conditions and photometric quantification of the formed chromophores, as methodically described by Molnir-Perl (Ed.) (2015), Journal of Chromatography Library 70: 405-444.

Dry Hydrogel Content in Hydrogel Suspension

The content of a hydrogel suspension was determined by successive washing of representative aliquots of the suspension in syringe reactors with PE frits with water and absolute ethanol and subsequent drying of the solid hydrogel portions in vacuum. The hydrogel content was calculated from the mass of the hydrogel residue per syringe and the respective aliquot volume of the hydrogel suspension.

MTS Load Quantification

The MTS load of an adequate hydrogel was determined by quantification of free thiols on the hydrogel by an Ellman assay after removal of the MTS groups by means of TCEP reduction. The determination was performed with aliquots of the appropriate MTS-hydrogel suspensions in syringe reactors with PE frits. By using the hydrogel content of the suspensions, the MTS load of the dry hydrogel was calculated.

Ellman Assay

The thiol content of a compound, which can either be soluble or insoluble in aqueous systems is determined by reaction of the free compound thiol groups with DTNB reagent in neutral pH and photometric determination of the released 5-thio-2-nitrobenzoic acid (TNB) as methodically described in G. L. Ellman (1959), Archives of Biochemistry and Biophysics 82: 70-77.

Reverse Ellman Assay

The maleimide content of a compound, which can either be soluble or insoluble in aqueous systems is determined by quenching the maleimide groups with an excess of 2-mercaptoethanol under neutral conditions. The amount of remaining, non-reacted thiol reagent is determined by an Ellman assay. The difference between the amount of 2-mercaptoethanol added in total and the residual thiol after reaction is used to calculate the maleimide content of the compound.

Protein Concentration Determination

Concentration determinations of protein solutions were performed on a Tecan Infinite M200 using UV-cuvette micro (neoLAB) and the following conditions: path length 1 cm; absorbance wavelength 280 nm; absorbance wavelength bandwidth 5 nm; reference wavelength 338 nm; reference wavelength bandwidth 25 nm; number of flashes 25. Extinction coefficient of HHC^{MET}: c=2.052 mL/(mg*cm). Concentrations of conjugate mixtures containing HHC^{MET} were determined by using the extinction coefficient of HHC^{MET}. Extinction coefficient of CTLA-4 mAB: E=1.53 mL/(mg*cm). Concentrations of conjugate mixtures containing CTLA-4 mAB were determined by using the extinction coefficient of CTLA-4 mAB.

### Example 1

Synthesis of Protected Diamino Alcohol 1c

1c was synthesized according to the following scheme:

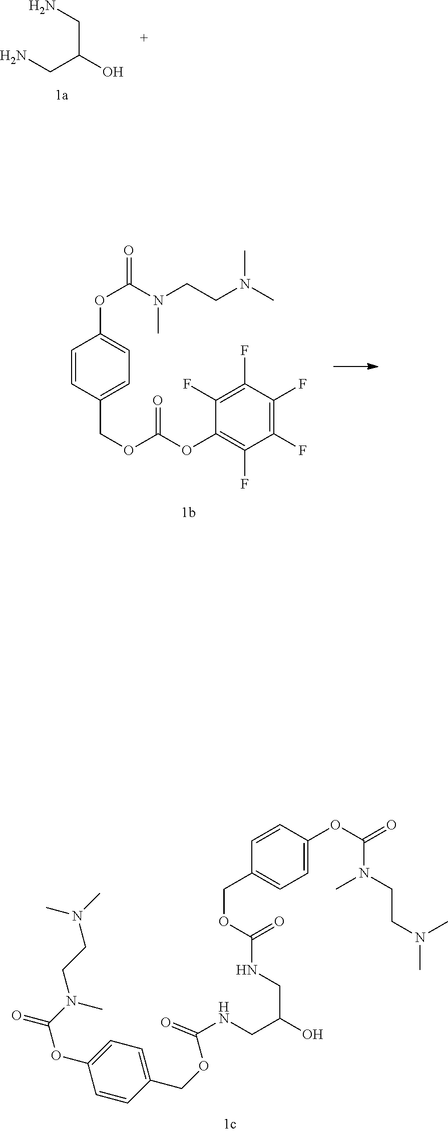 [SMILES: NCC(O)CN ; CN(C)CCN(C)C(=O)Oc1ccc(COC(=O)Oc2c(F)c(F)c(F)c(F)c2F)cc1 ; CN(C)CCN(C)C(=O)Oc1ccc(COC(=O)NCC(O)CNC(=O)OCc2ccc(OC(=O)N(C)CCN(C)C)cc2)cc1]

1b was synthesized according to WO 2018/175788 A1 Example TA and used as the TFA salt. 1b (352 mg, 0.61 mmol) was dissolved in acetonitrile (2.50 mL) and the solution cooled in an ice-bath. DIPEA (242 uL, 1.39 mmol) was added and the reaction was mixed. 1,3-diamino-2-propanol (25 mg, 0.28 mmol) was dissolved in acetonitrile (1.00 mL) and added to the reaction.

The reaction was mixed and incubated in the ice-bath.

A reaction control after 5 min indicated complete reaction.

After ca 15 min TFA (106 μL, 1.39 mmol) was added to the ice cooled reaction. The reaction was diluted with 4 ml water containing 0.1% TFA. The product 1c was purified by RP-HPLC.

Yield: 204 mg (84%, 2×TFA salt)

MS: m/z 647.34=[M+H]+, (calculated=647.34).

Synthesis of Linker Reagent 1i

1i was synthesized according to the following scheme:

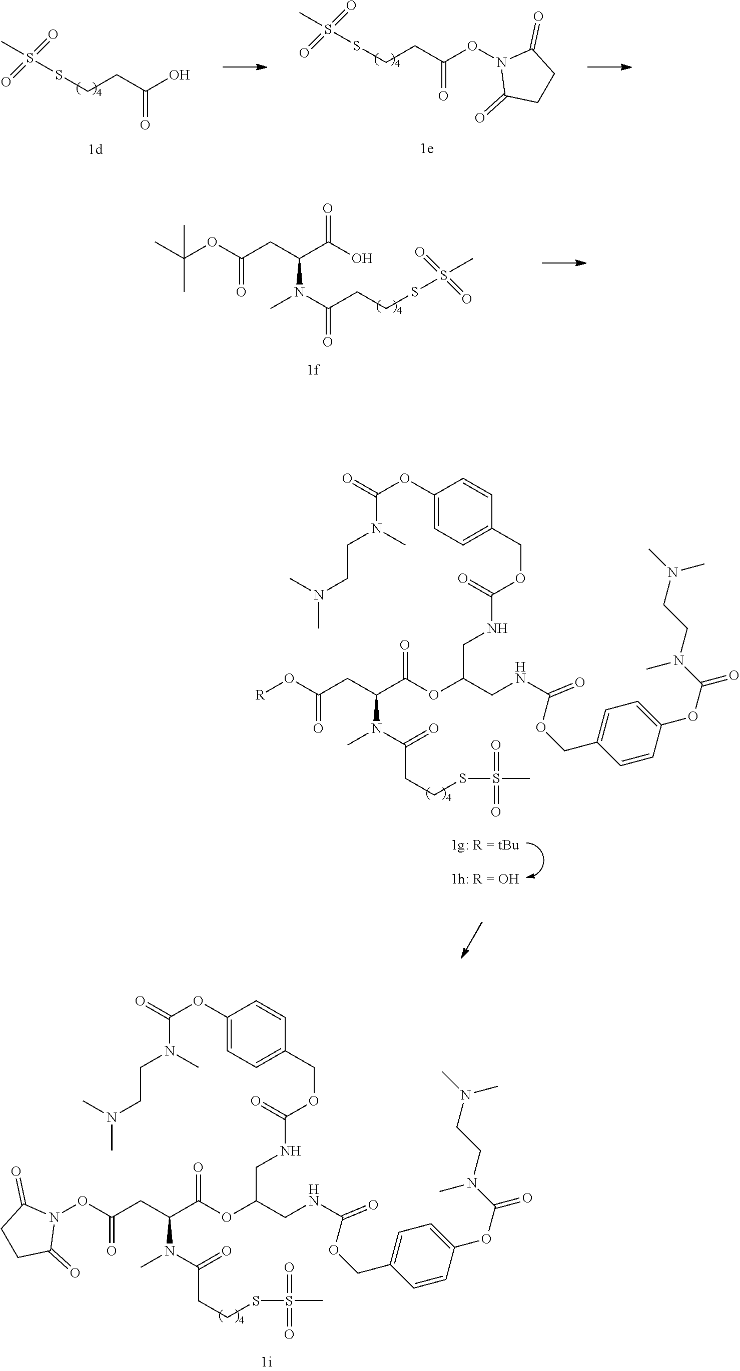

1d was synthesized according to WO 2018/175788 A1 Example 1E.

1d (800 mg, 3.53 mmol), HOSu (610 mg, 5.30 mmol) and EDC (1.02 g, 5.30 mmol) were dissolved in dichloromethane (9.6 ml). DIPEA (616 μl, 3.53 mmol) was added. After 1 h the reaction showed incomplete conversion to the product and after 75 min HOSu (203 mg, 1.77 mmol) and EDC (339 mg, 1.77 mmol) were added to the reaction. After 135 min the reaction was diluted with 100 ml ethyl acetate and was washed three times with 130 ml of 1 M hydrochloric acid. The organic phase was separated and dried over MgSO₄, filtered and concentrated under reduced pressure. The resulting crude was dried under high vacuum over night. The crude was purified by RP-HPLC yielding 1e.

Yield: 913 mg (80%)

MS: m/z 346.02=[M+Na]⁺, (calculated=346.04).

N-Me-Asp(OtBu)-OH (100 mg, 0.49 mmol) was suspended in dichloromethane (0.5 ml) and a solution of 1e (239 mg, 0.74 mmol) in dichloromethane (0.5 ml) was added. DIPEA (171 μL, 0.98 mmol) was added. After 95 min the reaction was quenched with 171 μl acetic acid and the volatiles removed in a stream of argon. The crude was purified by RP-HPLC yielding 1f.

Yield: 235 mg (97%)

MS: m/z 412.07=[M+H]⁺, (calculated=412.15). 1c (150 mg, 171 μmol) was dissolved in acetonitrile (4 mL). 1f (85 mg, 206 μmol) and DMAP (42 mg, 343 μmol) were added and dissolved. While stirring DIC (106 μL, 686 μmol) was added and the reaction was stirred for 30 min. The reaction was quenched with 4 ml 1% TFA in water and filtered. The filtrate was purified by RP-HPLC yielding 1g.

Yield: 151 mg (69%, 2×TFA salt)

MS: m/z 520.74=[M+2H]²⁺, (calculated=520.74).

1g (90 mg, 71 μmol) was dissolved in dichloromethane (1 mL) and the solution stirred vigorously in an open flask. TFA (1 mL) was added in one portion. The reaction was stirred for 45 min and the volatiles were removed in a stream of nitrogen. The residue was dissolved in 250 μl acetonitrile and precipitated in 10 ml diethylether. The reaction flask was rinsed with 250 μl anhydrous acetonitrile, which was added to the ether suspension. The suspension was centrifuged, and the supernatant removed. The precipitate was shortly dried in a stream of nitrogen yielding 1h (2×TFA salt).

1h was dissolved in 1 ml dichloromethane and 1 ml acetonitrile. HOSu (25 mg, 213 μmol), EDC (41 mg, 213 μmol) and DMAP (0.87 mg, 7.1 μmol) were added to the reaction. After 30 min the reaction volume was reduced to 1 ml in a stream of nitrogen. After 1 h HOSu (25 mg, 213 μmol) and EDC (41 mg, 213 μmol) were added. After 2 h 3 ml water containing 0.1% TFA were added to the reaction. The pH was adjusted to <pH 2 by addition of 60 μl 10% TFA in acetonitrile/water 1:1. The product was purified from the crude reaction mixture by RP-HPLC yielding 1i.

Yield: 83 mg (90%, 2×TFA salt)

MS: m/z 1081.42=[M+H]⁺, (calculated=1081.42).

### Example 2

Synthesis of Ubiquitin-Linker-PEG20k Conjugate 2b:

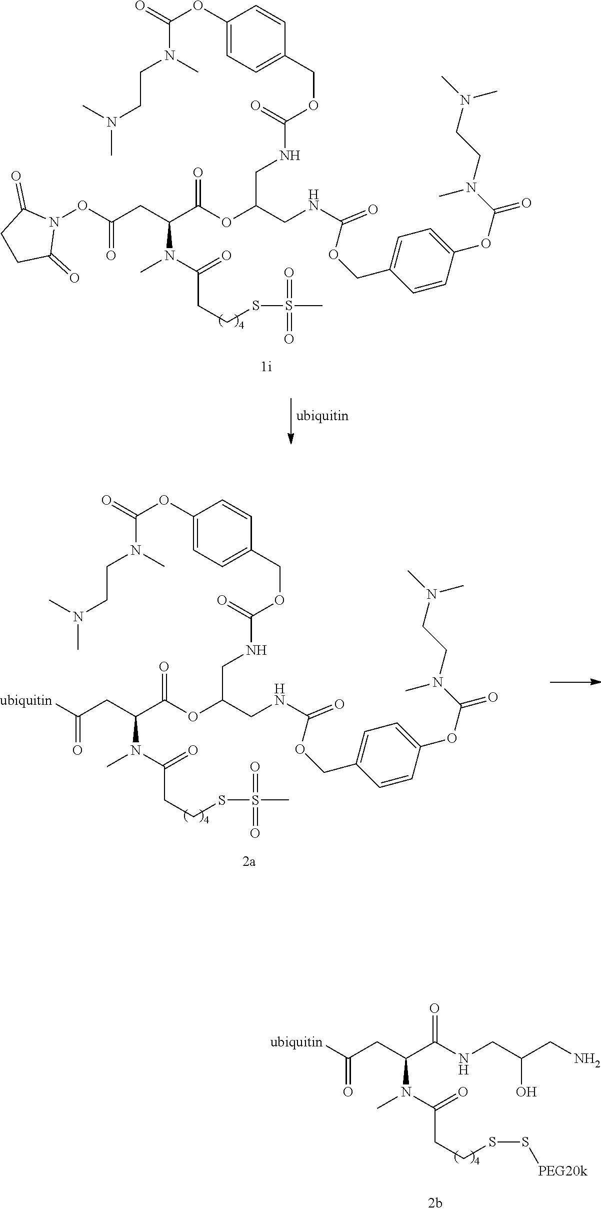

Ubiquitin from bovine erythrocytes in pH 7.4 60 mM sodium phosphate buffer (157 μl, 12.75 mg/ml) was mixed with a solution of 1i in DMSO (4.86 μl, 0.1 M). The reaction was incubated for 10 min at rt and quenched by addition of 39.3 μl pH 3 sodium succinate buffer (0.5M) shifting the pH of the reaction to about 4. To remove unconjugated linker from the protein conjugate mixture a buffer exchange to 5 mM sodium succinate pH 4 was performed using an Äkta pure system equipped with a GE HiTrap column at a flow rate of 2 ml/min. 1.5 ml of the product fraction were collected which contains ubiquitin and ubiquitin linker conjugates 2a. 9.3 mg of mPEG thiol 20 kDa (Sunbright ME-200SH) were dissolved in the product fraction and 225 μl pH 7.8 0.5 M sodium phosphate buffer containing 200 mM TriMED. were added to the reaction to facilitate cleavage of the protecting groups and rearrangement of the ester 2a to the amide 2b. The reaction was mixed and incubated for 20 h at 25° C.

Ubiquitin-linker-PEG20k monoconjugate was purified by ion exchange chromatography on a GE Healthcare source 15S 4.6/100 PE column connected to an Äkta pure system. The reaction was diluted 10 fold to a volume of 20 ml with water and the pH was brought to about 4 by addition of 50 μl 10% TFA in water. For the chromatography 20 mM sodium acetate, 10 mM methionine pH 4 was used as buffer A and 20 mM sodium acetate, 10 mM methionine 0.5 M NaCl pH 4.5 was used as buffer B with a gradient from 0-50% buffer B over 13 column volumes. The whole reaction volume was loaded in 4 5 ml injections on the column and eluted in a single run. Pure PEG monoconjugate was found in a 2 ml fraction. The fraction was buffer exchanged to PBS pH 7.4 using 3 HiTrap columns in series. The resulting 3 ml fraction was concentrated to 0.4 ml using Vivaspin Turbo 4 MWCO 5000 and stored at −20° C.

### Example 3

Synthesis of Protected Diamino Alcohols 3a-c

3a was synthesized according to the following scheme:

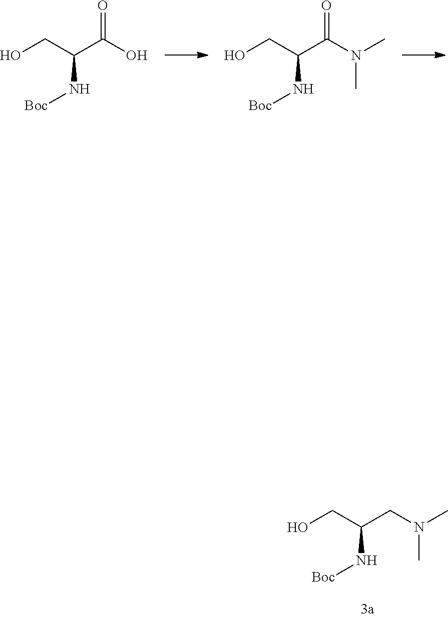 [SMILES: CC(C)(C)OC(=O)N[C@@H](CO)C(=O)O ; CN(C)C(=O)[C@H](CO)NC(=O)OC(C)(C)C ; CN(C)C[C@H](CO)NC(=O)OC(C)(C)C]

Dimethylamine solution in THF (536 μL, 2 M, 1.07 mmol) and dichloromethane (5 mL) were stirred at rt. DIPEA (204 μL, 1.17 mmol), Boc-Ser-OH (200 mg, 0.97 mmol), HOBt (198 mg, 1.46 mmol) and EDC (224 mg, 1.17 mmol) were added to the reaction. The reaction was stirred for 23 h. The reaction was diluted with 5 ml dichloromethane and washed with sat. bicarbonate, 5% citric acid, water, and brine (lx each). Each wash was back extracted with 5 ml DCM. The combined organic phase was dried (MgSO₄), filtered and concentrated in vacuo. The obtained Boc-Ser-NMe2 was used without further purification in the next step.

Yield: 140 mg (62%)

MS: m/z 232.96=[M+H]⁺, (calculated=233.15).

The crude from the last step (74 mg, 0.32 mmol) was dissolved in THF (0.4 mL) and toluene (0.2 mL) and stirred in an atmosphere of nitrogen in an ice-bath. Sodium bis(2-methoxyethoxy)aluminum hydride (70% w/w in toluene, 445 μL, 1.6 mmol) was added dropwise with gas evolution. After 4 h the reaction was quenched with 1 M aq. NaOH (0.65 mL). The reaction was stirred for 30 min at rt. The reaction was acidified with TFA (pH<2). The white precipitate was filtered off and washed with acetonitrile/water 1:1 containing 0.10% TFA (ca 1.5 ml). The filtrate was purified by RP-HPLC yielding 3a.

Yield: 61 mg (57%, TFA salt)

MS: m/z 219.17=[M+H]⁺, (calculated=219.17).

3b was synthesized according to the following scheme:

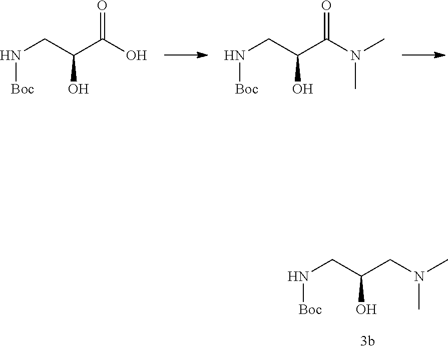 [SMILES: CC(C)(C)OC(=O)NC[C@H](O)C(=O)O ; CN(C)C(=O)[C@@H](O)CNC(=O)OC(C)(C)C ; CN(C)C[C@@H](O)CNC(=O)OC(C)(C)C]

Dimethylamine solution in THF (536 μL, 2 M, 1.07 mmol) and dichloromethane (5 mL) were stirred at rt. DIPEA (204 μL, 1.17 mmol), (S)-3-((tert-Butoxycarbonyl)amino)-2-hydroxypropanoic acid (200 mg, 0.97 mmol), HOBt (198 mg, 1.46 mmol) and EDC (224 mg, 1.17 mmol) were added to the reaction. The reaction was stirred for 19 h. The reaction was diluted with 5 ml DCM and washed with sat. bicarbonate, 5% citric acid, water, and brine (lx 5 ml, each). Each wash was back extracted with 5 ml DCM. The organic phase was dried (MgSO4), filtered and concentrated in vacuo. The obtained intermediate was used without further purification in the next step.

Yield: 204 mg (90%)

MS: m/z 233.15=[M+H]⁺, (calculated=233.15).

The crude from the last step (204 mg, 0.88 mmol) was dissolved in THF (1 mL) and toluene (0.5 mL) and stirred in an atmosphere of nitrogen in an ice-bath. Sodium bis(2-methoxyethoxy)aluminum hydride (70% w/w in toluene, 1.22 mL, 4.39 mmol) was added dropwise with gas evolution. After 3 h the reaction was quenched with 1 M aq. NaOH (1.63 mL). The reaction was stirred for 30 min at rt. The water layer was extracted 3× with 5 ml diethyl ether. The ether layer was extracted with 10 ml 0.64 M aq. HCl and the product was isolated from the aq. acidic phase by RP-HPLC yielding 3b.

Yield: 173 mg (59%, TFA salt)

MS: m/z 219.17=[M+H]⁺, (calculated=219.17).

3c was synthesized according to the following scheme:

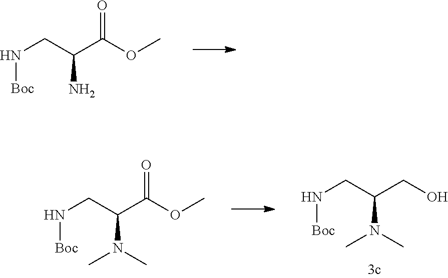 [SMILES: COC(=O)[C@@H](N)CNC(=O)OC(C)(C)C ; COC(=O)[C@H](CNC(=O)OC(C)(C)C)N(C)C ; CN(C)[C@H](CO)CNC(=O)OC(C)(C)C]

H-Dpr(Boc)-OMe HCl salt (200 mg, 0.79 mmol) was dissolved in MeOH (9.4 mL) and aq formaldehyde (37% w/w, 175 μL) was added to the solution. The reaction was stirred for 10 min and sodium cyanoborohydride (148 mg, 2.36 mmol) was added in one portion. The reaction was stirred for 2.5 h. Aq. formaldehyde (88 μL) and sodium cyanoborohydride (74 mg, 1.18 mmol) were added to the reaction. After 5 h the reaction was quenched with a solution prepared from 5 ml sat. bicarbonate solution and 5 ml water. The mixture was stirred overnight in a fume hood in an open flask. The aqueous phase was extracted with DCM (4×20 ml). The combined organic phase was dried (MgSO₄), filtered and concentrated in vacuo. The obtained intermediate was used without further purification in the next step.

Yield: 161 mg (83%)

MS: m/z 247.17=[M+H]⁺, (calculated=247.17).

The crude from the last step (160 mg, 0.65 mmol) was dissolved in THF (1.5 mL) and the reaction cooled in an ice-bath. Lithium aluminium hydride solution (1 M in THF) (1.3 mL, 1.3 mmol) was drop-wise added with stirring (gas formation). After 1 h the reaction was diluted with 5 ml diethyl ether and 50 μl water were added dropwise. After the gas formation subsided, 50 μl 4 M NaOH were added following 150 μl water. The reaction was stirred for 30 min at rt. MgSO₄ was added and the reaction stirred for 15 min. The solids were filtered off and washed with 5 ml diethyl ether. The volatiles were removed in a stream of nitrogen. The product was purified by RP-HPLC yielding 3c.

Yield: 158 mg (73%, TFA salt)

MS: m/z 219.17=[M+H]⁺, (calculated=219.17).

### Example 4

Synthesis of Ac—N-Me-Asp(OBzl)-OH

Fmoc-N-Me-Asp(OBzl)-OH (500 mg, 1.09 mmol) was dissolved in THF (5 mL). DBU (325 μL, 2.18 mmol) was drop-wise added with stirring. A suspension formed. After 20 min a solution of N-acetoxysuccinimide (342 mg, 2.18 mmol) in THF (5 mL) was added to the reaction. The suspension turned slowly into a solution. After 50 min the reaction solution was diluted with DCM (20 ml) and extracted with sat. sodium bicarbonate (30 ml). The water phase was washed again with DCM (20 ml). The organic phase was discarded. Ethyl acetate (30 ml) was added to the bicarbonate phase which was acidified with conc. HCl to <pH 2. The organic phase was washed furthermore with 0.1 M HCl (2×30 ml). The organic phase was dried (MgSO₄), filtered and concentrated in vacuo to yield Ac—N-Me-Asp(OBzl)-OH (4).

Yield: 299 mg (99%)

MS: m/z 280.12=[M+H]⁺, (calculated=280.12).

### Example 5

Synthesis of Linker Reagents 5e-h

5e-h were synthesized according to the following scheme:

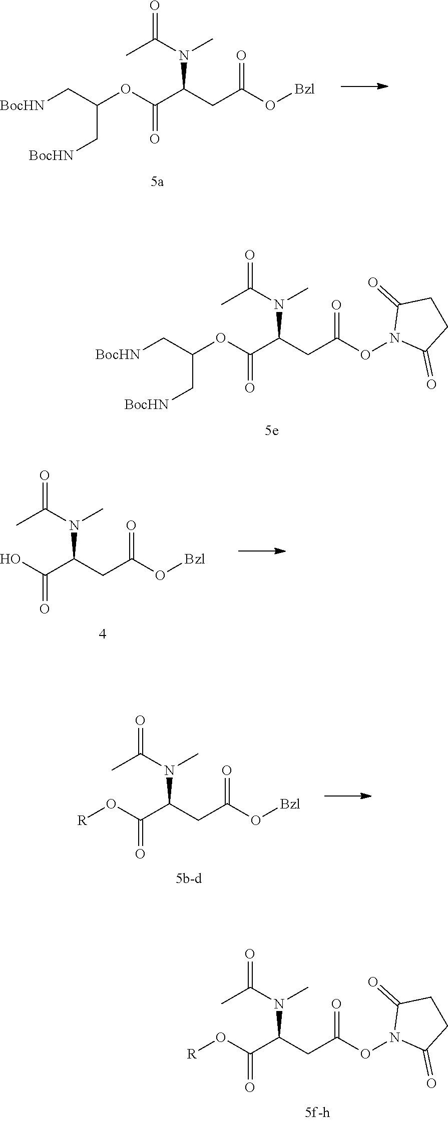

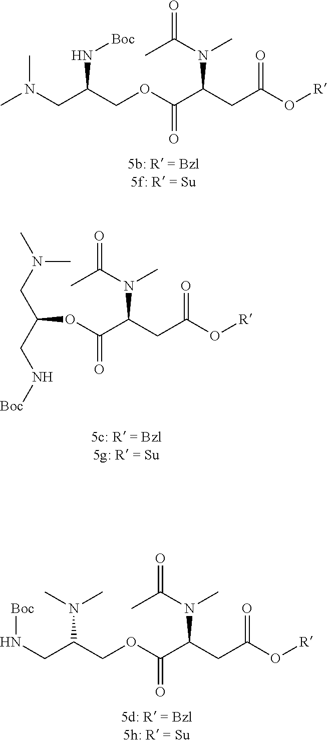

Synthesis of 5e:

Fmoc-N-Me-Asp(OBzl)-OH (138 mg, 0.30 mmol) was dissolved in DCM (1 mL) and N,N′-bis-Boc-2-hydroxy-propylene diamine (105 mg, 0.36 mmol) and EDC (86 mg, 0.45 mmol) were added with stirring. Catalytic amounts of DMAP were added. After 2.5 h the reaction was diluted with 10 ml of DCM and washed twice with citric acid, twice with sat. bicarbonate solution and once with brine (10 ml each). The organic phase was dried (MgSO₄), filtered and reduced to 2 ml volume in vacuo.

DBU (90 μL, 0.60 mmol) was added with stirring. After 15 min the reaction was complete and acetic anhydride (142 μL, 1.50 mmol) was added with stirring. After 30 min, the reaction was acidified with 100 μl acetic acid and the solvent was removed in a stream of nitrogen.

Intermediate 5a was purified by RP-HPLC.

Yield: 107 mg (65%)

MS: m/z 552.29=[M+H]⁺, (calculated=552.29).

5a (107 mg, 0.19 mmol) was dissolved in THF (2 mL) and palladium on activated charcoal 10% Pd basis (41 mg, 0.04 mmol) was added and the reaction was stirred vigorously under an atmosphere of hydrogen. The reaction mixture was diluted with THF (8 ml) and filtered over a 0.2 μm PTFE syringe filter. TSTU (117 mg, 0.39 mmol) and DIPEA (67 μL, 0.39 mmol) were added. The suspension was stirred at rt. After 16 h the suspension was filtered. The filtrate was concentrated in vacuo. The residue was partitioned between ethyl acetate and pH 7 sodium phosphate buffer (100 mM). The organic phase was washed 2 times with pH 7 buffer and 1× with brine. The organic phase was dried (MgSO₄), filtered and concentrated in vacuo. The oily residue was dissolved in DCM and concentrated in vacuo yielding 5e as white foam.

Yield: 106 mg (98%)

MS: m/z 559.26=[M+H]⁺, (calculated=559.26).

Synthesis of 5b-d:

5b: 4 (47 mg, 167 μmol) was dissolved in DCM (1 ml). The solution was added to 3a (61 mg 183 μmol). The reaction was stirred and EDC (35 mg, 183 μmol) was added. DMAP (2 mg, 17 μmol) was added. After 1 h EDC (35 mg, 183 μmol) and DMAP (2 mg, 17 μmol) were added.

After 2 h. the volatiles were removed in a stream of nitrogen. 5b was purified by RP-HPLC.

Yield: 48 mg (48%, TFA salt)

MS: m/z 480.27=[M+H]⁺, (calculated=480.27).

5c: Accordingly to 5b, 5c was synthesized using 4 (50 mg, 0.18 mmol) and 3b (65 mg, 0.2 mmol). EDC (76 mg, 0.39 mmol) and DMAP (2 mg, 0.02 mmol) were added in one portion with 18 h reaction time.

Yield: 39 mg (37%, TFA salt)

MS: m/z 480.27=[M+H]⁺, (calculated=480.27).

5d was synthesized following the procedure and equivalents for 5c but using 3c instead of 3b.

Yield: 30 mg (28%, TFA salt)

MS: m/z 480.27=[M+H]⁺, (calculated=480.27).

Synthesis of 5f-h:

5f: 5b (48 mg, 100 μmol) and Palladium on activated charcoal 10% Pd basis (21 mg) were suspended in THF (2 ml) in an atmosphere of nitrogen. The vessel was charged with hydrogen and kept in an atmosphere of hydrogen for 2.5 h. The reaction mixture was filtered over a 0.2 μm PTFE syringe filter and rinsed with THF (4 ml). TSTU (60 mg, 200 μmol) and DIPEA (35 μl, 200 μmol) were added to the filtrate. The suspension was stirred at rt for 16 h. The volatiles were removed and the product purified by RP-HPLC yielding 5f.

Yield: 36 mg (60%, TFA salt)

MS: m/z 487.24=[M+H]⁺, (calculated=487.24).

5g: Accordingly to 5f, 5g was synthesized using 5c (39 mg, 36 μmol), Pd on charcoal (4 mg), THF (2 ml) and 1 h reaction time. The reaction was filtered over a 1 μm PTFE syringe filter and rinsed with DCM (2 ml). TSTU (40 mg, 132 μmol) and DIPEA (46 μl, 265 μmol) were added to the filtrate. The suspension was stirred for 1 h. The volatiles were removed, and the product purified by RP-HPLC yielding 5g.

Yield: 25 mg (63%, TFA salt)

MS: m/z 487.24=[M+H]⁺, (calculated=487.24).

5h was synthesized following the procedure for 5g, but using 5d (30 mg, 51 μmol) instead of 5c and scaling the reagents accordingly.

Yield: 19 mg (61%, TFA salt)

MS: m/z 487.24=[M+H]⁺, (calculated=487.24).

### Example 6

Synthesis of AcAKF-OH Linker Conjugates 6a-d from Linker Reagents 5e-h

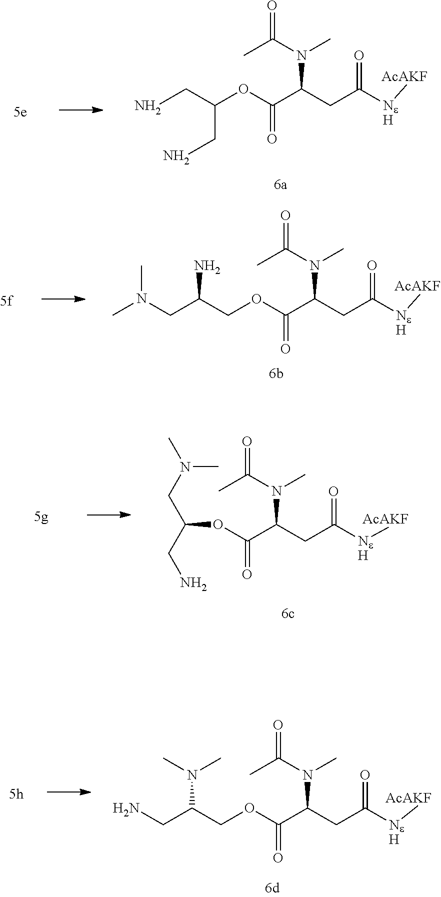

AcAKF tripeptide was synthesized on 2-chlorotritylchloride resin using Fmoc amino acids Fmoc-Ala-OH, Fmoc-Lys(ivDDe)-OH, Fmoc-Phe-OH. The N-terminus of the peptide was acetylated on resin using acetic anhydride/DIPEA and ivDDE was cleaved using hydrazine. Linkers 5e-h were coupled to the free side chain amine of Lys on resin using 8-9 mg of the peptide loaded resin, 2 eq of the respective linker and 3 eq of DIPEA in DMF. The resin was agitated for 30 min. The resin was washed 5 times with DMF and 5× with DCM, before the Boc protected peptide was cleaved from the resin with 20% HFIP in DCM. The Boc protected peptides were purified by RP-HPLC. Cleavage of the Boc protecting group(s) was performed with TFA/DCM 1:1. The volatiles were removed; the residue was dissolved in acetonitrile and the peptide linker conjugates were isolated after ether precipitation as TFA salts.

6a: Yield: 3 mg (37%, 2×TFA salt) MS: m/z 650.30=[M+H]⁺, (calculated=650.35).

6b Yield: 2 mg (25%, 2×TFA salt) MS: m/z 678.40=[M+H]⁺, (calculated=678.38).

6c Yield: 2.4 mg (34%, 2×TFA salt) MS: m/z 678.40=[M+H]⁺, (calculated=678.38).

6d Yield: 2.4 mg (34%, 2×TFA salt) MS: m/z 678.39=[M+H]⁺, (calculated=678.38).

### Example 7

In Vitro Release Kinetics

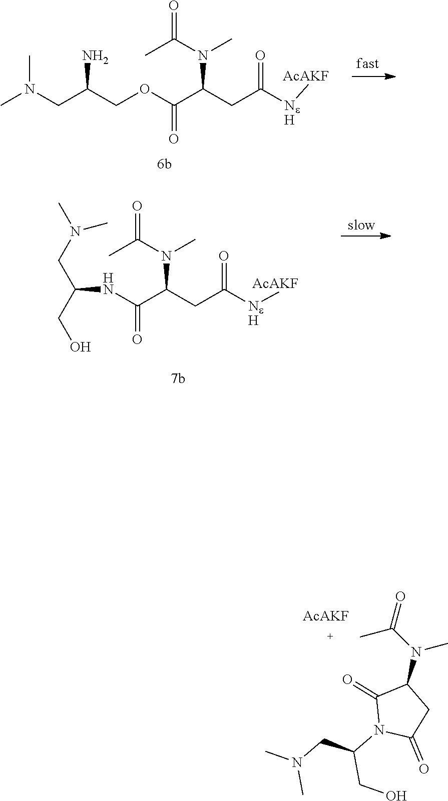

The cleavage rate of tripeptide AcAKF from conjugates 6a-d was monitored at pH 7.4 and 37° C. in aqueous buffer (pH 7.4 60 mM sodium phosphate) by LCMS (UV detection). In a first step the rearrangement of the linker moiety takes place (exemplary depicted for conjugate 6b above) within minutes, before the disappearance of the conjugate 7b over time was determined and fitted with curve fitting software to obtain the half-life of the slow release.

| Compound | t_{1/2} (pH 7.4) |
|---|---|
| 6a | 16 d |
| 6b | 27 d |
| 6c | 15 d |
| 6d | 3.0 d |

### Example 8

In Vitro Release Kinetics

The cleavage rate of the reversible bond from conjugate 2b was monitored at pH 7.4 and 37° C. in aqueous buffer (PBS pH 7.4) by LCMS (UV detection). The peak area percentage of increasing free ubiquitin over time was determined and fitted with curve fitting software to obtain the half-life of the release.

Compound t_{1/2} (pH 7.4)

2b 30 d

### Example 9

Synthesis of Linker Reagent 9g

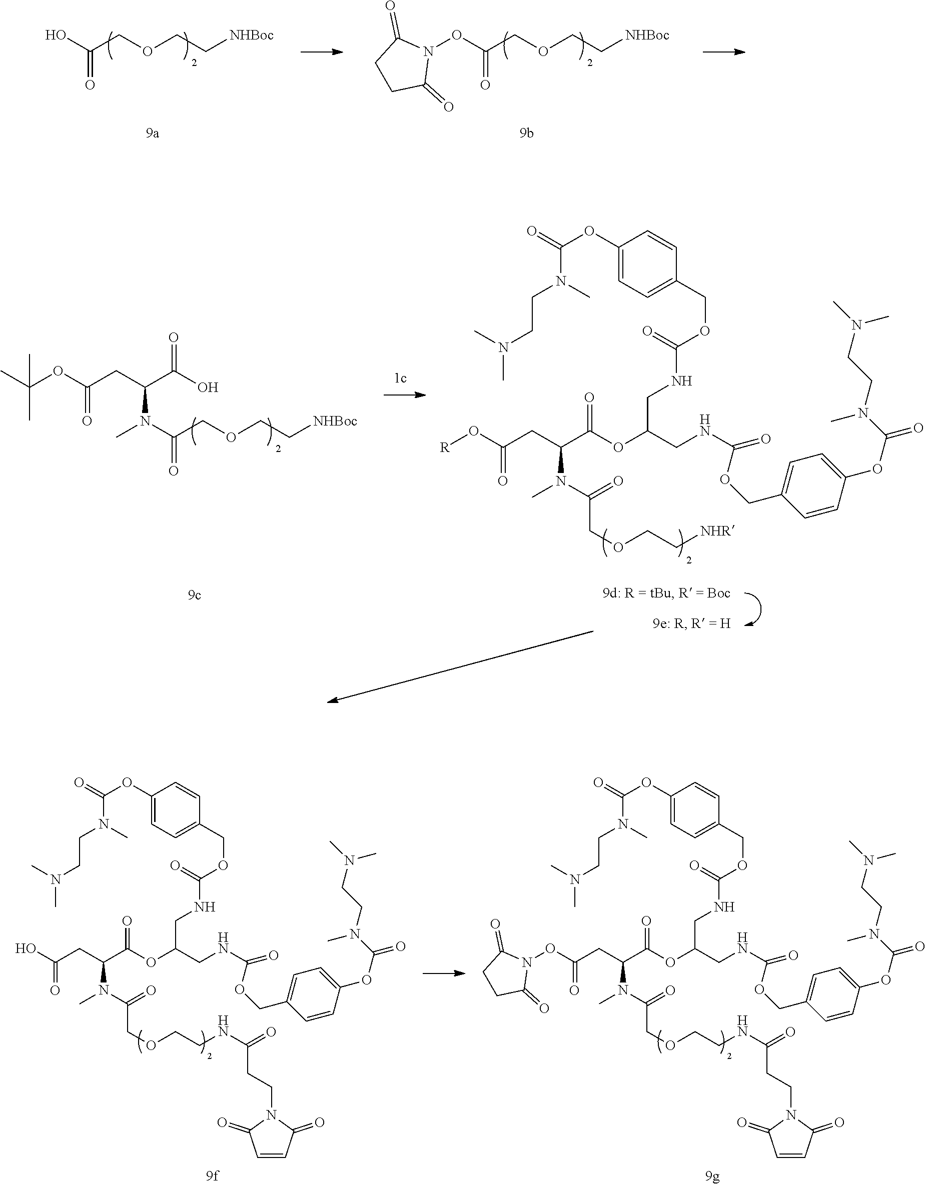

9a (1.5 g, 5.7 mmol) was dissolved in THF (37.5 mL). TSTU (2.6 g 8.6 mmol) and DIPEA (3.97 mL, 22.8 mmol) were added. Upon stirring a turbid suspension was formed. The mixture was stirred for 22 h, TSTU (1.7 g 5.5 mmol), DIPEA (2 mL, 11.5 mmol) and DMF (13 mL) were added and the color of the reaction turned dark brown. After a total of 26 h the reaction mixture was diluted with 350 mL of ethyl acetate and washed with 2×200 mL 0.1N HCl and 1× with 100 mL of brine. The organic phase was dried over Na₂SO₄ and evaporated. The residue was dried under high vacuum overnight. The product was purified using flash chromatography yielding 9b as colorless oil.

Yield: 1.65 g (81%)

MS: m/z 361.17=[M+H]⁺, (calculated=361.16).

9b (1.65 g, 4.58 mmol) was dissolved in DCM (11.6 mL) and N-Me-L-Asp(tBu)-OH (932 mg, 4.59 mmol) and DIPEA (1.6 mL, 9.2 mmol) were added. The white suspension was stirred at RT. The mixture slowly became a light-yellow solution over time.

Acetic acid (786 μL, 13.7 mmol) was added after 1 h. The solvent was evaporated and the product purified by RP-LPLC yielding 9c.

Yield: 1.77 g (86%)

MS: m/z 449.15=[M+H]⁺, (calculated=449.25).

9c (1.23 g, 2.74 mmol) and 1c (1.99 g, 2.28 mmol) were dissolved in acetonitrile (53 mL). DMAP (557 mg, 4.56 mmol) was added under stirring and to the resulting solution DIC (1.41 mL, 9.12 mmol) was given. After 1 h 0.7 ml TFA were added and the solvent removed in vacuo. The product was purified by RP-LPLC yielding 9d.

Yield: 2.33 g (78%, 2×TFA salt)

MS: m/z 1077.65=[M+H]⁺, (calculated=1077.57).

9d (2.33 g, 1.78 mmol) was dissolved in DCM (10 mL). TFA (10 mL, 131 mmol) was added under stirring. After 45 min the solvent was evaporated and the residue was co-evaporated with 50 mL of DCM. The residue was dried under high vacuum overnight yielding 2.90 g of 9e, which was used without further purification. 9e was dissolved in acetonitrile (68 mL) and 3-maleimidopropionic acid N-hydroxysuccinimide ester (1.19 g, 4.45 mmol) was added under stirring. DIPEA (3.1 mL, 17.8 mmol) was added. After 80 min the reaction was quenched by addition of TFA (1.36 mL, 17.8 mmol). The reaction was concentrated in vacuo to a volume of 40 ml and the product purified by RP-LPLC yielding 9f.

Yield: 1.73 g (75% over 2 steps, 2×TFA salt)

MS: m/z 1072.60=[M+H]⁺, (calculated=1072.49).

9f (1.73 g, 1.33 mmol) was dissolved in acetonitrile (17 mL) and EDC (767 mg, 4 mmol), HOSu (462 mg, 4 mmol) and DMAP (19 mg, 0.15 mmol) were added under stirring. After 1 h 30 min the reaction was quenched by addition of TFA (100 μL, 1.3 mmol) and the reaction was concentrated in vacuo to a volume of 8.5 mL and the product purified by RP-LPLC yielding 9g.

Yield: 1.36 g (73%, 2×TFA salt)

MS: m/z 1169.71=[M+H]⁺, (calculated=1169.50).

### Example 10: Preparation of Amine-HAs 10a and 10b

Hyaluronic acid sodium salt (90-130 kDa, 504 mg, 1.25 mmol COOH, 1.00 eq.) was dissolved in 100 mM MES 400 mM 1,3-diaminopropane buffer pH 5.5 (62.5 mL) under vigorous stirring. HOBt (573 mg; 3.74 mmol, 3.00 eq.) and EDC (223 mg; 1.17 mmol, 0.93 eq.) were added. The suspension was stirred at ambient temperature overnight. Sodium acetate trihydrate (8.48 g) was added, whereupon the suspension turned into a solution. The crude amine-modified HA was precipitated by addition of absolute ethanol, washed with 80% (v/v) ethanol and absolute ethanol and was dried under high vacuum for 1 h. The pellets were dissolved in water (40 mL) to form a clear solution. 4 M NaOH (13.3 mL) was added and the solution was stirred at ambient temperature for two hours before acetic acid (3.05 mL) was added. The product was precipitated by addition of absolute ethanol, washed with 80% (v/v) ethanol and absolute ethanol and was dried under high vacuum to give amine-functionalized HA 10a as acetate salt. The amine content of the material was determined by an OPA assay.

Yield: 432 mg (acetate salt, amine-content: 0.253 mmol/g, 10.4% DS)

Amine-HA 10b was prepared accordingly to the procedure described above, only using a different amount of EDC (95.8 mg; 0.50 mmol, 0.404 eq.).

Yield: 449 mg (acetate salt, amine-content: 0.114 mmol/g, 4.6% DS)

### Example 11: Preparation of Thiol-HA 11 from Amine-HA 10a

Amine-functionalized HA 10a (400 mg, 0.101 mmol amines, 1.0 eq.) was dissolved in 100 mM HEPES buffer pH 8.4 (33.25 mL). A freshly prepared solution of SPDP (318 mg, 1.02 mmol, 10.1 eq.) in acetonitrile (18 mL) was added to the mixture while stirring. The mixture was stirred at ambient temperature for 120 minutes before a freshly prepared solution of TCEP (582 mg, 2.03 mmol, 20.1 eq.) in water (5.13 mL) was added to the reaction mixture. The solution was stirred for one hour at ambient temperature before 1 M sodium acetate buffer pH 5.5 (56.4 mL) was added. The product was collected by addition of absolute ethanol and centrifugation. After washing with 80% (v/v) ethanol, absolute ethanol and drying in high vacuum for five hours, crude thiol-HA was obtained as white solid. The crude material was dissolved in 1% acetic acid (40 mL) by vigorous stirring under an argon atmosphere. 1 M sodium acetate buffer pH 5.5 (40 mL) was added to the solution and the resulting mixture was filtered through a 0.22 μm PES bottle-top filter. The product was precipitated from the filtrate by addition of absolute ethanol and centrifugation. After washing with 80% (v/v) ethanol and absolute ethanol, the material was dried in high vacuum for six hours to give thiol-HA 11 as off-white pellets. Thiol content was determined via Ellman assay.

Yield: 366 mg (thiol-content: 0.209 mmol/g)

### Example 12: Preparation of Maleimide-HA 12 from Amine-HA 10b

Amine-functionalized HA 10b (443 mg, 0.05 mmol amines, 1.0 eq.) was dissolved in 100 mM HEPES buffer pH 7.4 (44.25 mL). A freshly prepared solution of 3-maleimidopropionic acid NHS ester (134 mg, 0.49 mmol, 10.0 eq.) in acetonitrile (9.7 mL) was added to the mixture while stirring. The mixture was stirred at ambient temperature for 60 minutes before 1 M sodium acetate buffer pH 5.5 (54 mL) was added. The product was collected by addition of absolute ethanol and centrifugation. After washing with 80% (v/v) ethanol, followed by washing with absolute ethanol, the material was stored at −20° C. overnight and was dried in high vacuum for two hours the next day to yield crude maleimide-HA as white solid. The crude material was dissolved in 1% acetic acid (44.25 mL) by vigorous stirring. 1 M sodium acetate buffer pH 5.5 (54 mL) was added to the solution and the resulting mixture was filtered through a 0.22 μm PES bottle-top filter. The product was precipitated from the filtrate by addition of absolute ethanol and centrifugation. After washing with 80% (v/v) ethanol and absolute ethanol, the material was dried in high vacuum for six hours to give maleimide-HA 12 as white pellets. Maleimide content was determined via reverse-Ellman assay.

Yield: 376 mg (maleimide-content: 0.109 mmol/g)

### Example 13: Preparation of Crosslinked HA Microparticles with Free Thiols 13

Thiol-HA 11 (90.5 mg) was dissolved in 200 mM MES, 3 mM EDTA buffer pH 5.5 (3015 μL) by vigorous shaking under an argon atmosphere to produce a 30 mg/mL solution of the compound in buffer (solution A). Maleimide-HA 12 (70.7 mg) was dissolved in 200 mM MES, 3 mM EDTA buffer pH 5.5 (2355 μL) by vigorous shaking to produce a 30 mg/mL solution of the compound in buffer (solution B). In a 2 mL Eppendorf tube, equipped with a magnetic stirring bar, 200 mM MES, 3 mM EDTA buffer pH 5.5 (94.2 μL) was mixed with solution A (717.7 μL) and solution B (688.1 μL) under vigorous shaking. For gelling, the mixture was left standing at r.t. under an argon atmosphere overnight. The gel was transferred into a 5 mL Luer-Lock syringe to which a line of a male/female Luer Lock adapter, a 2×1 mm PTFE o-ring, a 144 μm stainless steel mesh (3.8 mm diameter), a 2×1 mm PTFE o-ring, a male/female Luer Lock adapter, a 2×1 mm PTFE o-ring, a 144 μm stainless steel mesh (4 mm diameter), a 2×1 mm PTFE o-ring and a male/female Luer Lock adapter was connected. The gel portion in the syringe was passed through the two 144 μm stainless steel meshes into 200 mM MES, 3 mM EDTA buffer pH 5.50 in a 15 mL Falcon tube. The hydrogel was successively washed with 3 mM EDTA buffer pH 5.5 followed by 200 mM succinate, 3 mM EDTA buffer pH 4.0 and 200 mM succinate, 3 mM EDTA, 0.5% Tween 20 buffer pH 4.0 by shaking, centrifugation and supernatant removal. After the last washing step, the volume of the gel suspension was adjusted to 10 mL with 3 mM EDTA, 0.5% Tween 20 buffer pH 4.0 in a 15 mL Falcon tube to yield the cross-linked HA with free thiol groups as colorless and almost completely transparent suspension. The thiol content of the hydrogel suspension was determined by Ellman assay.

### Example 14: Preparation of CTLA-4 mAB-Linker Conjugate Mixture 14

204.13 mL of CTLA-4 mAB at 5.341 mg/mL in 26 mM Tris-HCl, 100 mM NaCl, 55 mM mannitol, 0.1 mM pentetic acid (DTPA), 0.01% Tween80, pH 7.0 was used in this example. The mAB was buffer exchanged to 30 mM sodium phosphate, pH 7.4, concentrated, and the protein concentration was adjusted to of 9.74 mg/mL. 103.14 mL mAB solution were prepared.

3 mol eq. (218.6 μL) of linker reagent 9g (100 mM stock solution in DMSO) were added to the protein solution. The reaction mixture was mixed carefully and incubated for 5 min at ambient temperature yielding a mixture of unmodified CTLA-4 mAB and the protected CTLA-4 mAB-linker conjugates (e.g. monoconjugate, bioconjugate) 14.

A pH shift to about pH 4 was achieved by addition of 0.12 vol. eq. (12.4 mL) of 0.5 M succinic acid, pH 3.0 with respect to the volume of the mAB solution (103.1 mL), and the solution was mixed carefully. 14 was purified by cation exchange chromatography using an Aekta pure system equipped with an Eshmuno CPX column (8 mm ID×200 mm length, CV=10 mL) with 20 mM succinic acid, pH 5.5 as mobile phase and a linear salt gradient elution with sodium chloride (0-60% 20 mM succinic acid, 1 M NaCl, pH 5.5 in 15 CV) at a flow rate of 4.0 mL/min. Three runs with ˜39 mL injection volume (˜337 mg) per run were performed and 119.27 mL of 14 was collected at a concentration of 7.32 mg/mL.

To determine the content of reactive maleimides 20 μl of 14 were PEGylated with 20 kDa PEG thiol and subsequent SE-HPLC analysis was performed. After peak integration of the SE chromatogram a total maleimide content of 41% was determined for 14.

After analysis and overnight storage at 4° C., 118.38 mL of 14 were adjusted to a final concentration of 5 mM EDTA and 0.01% Tween20 with 1/19 vol. eq. of 20 mM succinic acid, 100 mM EDTA, 0.2% Tween20, pH 5.5 (6.2 mL) with respect to the volume of 118.38 mL and the solution was shaken carefully. The sample was filtered using one qpore Plastic vacuum filter (PVDF membrane) with a pore size of 0.22 μm.

122.67 mL of 14 at a concentration of 7.82 mg/mL were obtained.

### Example 15: Synthesis of Transient CTLA-4 mAB-Linker-Hydrogel Prodrug 15b

Conjugation of CTLA-4 mAB/protected CTLA-4 mAB-linker conjugate mixture 14 to thiol functionalized, crosslinked HA hydrogel 13 was performed by addition of mixture 14 to 1.5 mol. hydrogel 13 with respect to determined total maleimide content of 41% (4 μM).

7.5 mL of hydrogel suspension prepared according to example 13 (4.22 mg/mL nominal gel content with a thiol content of 200.8 μM) in 20 mM succinic acid, 150 mM NaCl, 3 mM EDTA, 0.1% Tween20, pH 4.0 were transferred into a 15 mL Falcon tube. Four 15 mL Falcon tubes were prepared like this in total. The hydrogel particles were sedimented by centrifugation at 4000 rcf for 1 minute and the supernatant was removed by pipetting. Washing of the particles was accomplished via five cycles of washing steps, which included addition of 10 mL 20 mM succinic acid, 5 mM EDTA, 0.01% Tween20, pH 5.5 buffer, centrifugation at 1000 rcf for 1 minute and careful removal of the supernatant by pipetting. After the last washing step, each of the four falcon tubes was filled up to a nominal total volume of suspension of 4 mL with above mentioned buffer. From each Falcon tube 2.6 mL of the hydrogel suspension were transferred into a fresh 50 mL Falcon tube resulting in four Falcon tubes each containing 2.6 mL of washed hydrogel suspension.

122.62 mL of 14 (c=7.82 mg/mL, 958.3 mg) at pH 5.5 were divided in four parts and approx. 33 mL were added to each of the four 50 mL Falcon tubes containing the hydrogel suspension described above. The resulting suspensions were mixed end-over-end and incubated at ambient temperature under gentle agitation overnight yielding protected transient CTLA-4 mAB-linker hydrogel prodrug 15a.

The hydrogel suspension was centrifuged at 1000 rcf for 1 minute and left standing for 3 minutes. The supernatants after the hydrogel loading were transferred in a 250 mL Corning bottle by pipetting. The hydrogel was combined in one 50 mL Falcon tube.

The hydrogel was first washed seven times with 30 mL 10 mM IAA in 30 mM sodium phosphate, 50 mM TriMED, 0.01% Tween20, pH 7.4. Afterwards, 30 mL 10 mM IAA in 30 mM sodium phosphate, 50 mM TriMED, 0.010% Tween20, pH 7.4 were added to hydrogel and incubated at ambient temperature under gentle agitation for 1 h. Removal of IAA blocking solution was accomplished via ten cycles of washing, which included addition of 30 mL 30 mM sodium phosphate, 50 mM TriMED, 0.01% Tween20, pH 7.4 buffer, centrifugation at 1000 rcf for 1 minute and careful removal of the supernatant by pipetting after 3 minutes resting.

Afterwards, for deprotection of the protected transient CTLA-4 mAB-linker hydrogel prodrug 15a, 30 mL 30 mM sodium phosphate, 50 mM TriMED, 0.01% Tween20, pH 7.4 buffer were added to the sedimented hydrogel and the resulting suspension was incubated at 25° C. overnight yielding transient CTLA-4 mAB-linker hydrogel prodrug 15b.

Final formulation of 15b was performed by washing the hydrogel ten times with 20 mM succinic acid, 10 w % α-α-D-trehalose, 0.01% Tween20, pH 5.5.

### Example 16: In Vitro Release Kinetics for 15b

25 mg of 15b (corresponding to approximately 0.45 mg protein) were transferred in a sterile, 1.5 mL Eppendorf tube. Eight tubes were prepared in total. 1 mL 60 mM sodium phosphate, 3 mM EDTA, 0.01% Tween20, pH 7.4 was added to each tube, which was subsequently mixed end-over-end and incubated without agitation for 5 minutes. The supernatant was removed to a final volume of 0.5 mL suspension per vial. The suspensions were incubated at 37° C. in a water bath. After different time intervals, one vial was removed from 37° C., centrifuged and the supernatant was analyzed by A280 measurement and SE-HPLC at 215 nm. The relative amount of released CTLA-4 mAB based on concentration determination of the supernatant with respect to the total amount of CTLA-4 mAB was recorded.

Release Kinetics from 15b:

| | CTLA-4 mAB | CTLA-4 mAB |
|---|---|---|
| t [d] | release [μg] | release [%] |
| 1.92 | 25.9 | 5.5 |
| 8.92 | 83.2 | 18.5 |
| 18.92 | 149.9 | 33.0 |
| 27.92 | 189.4 | 42.9 |
| 39.92 | 226.2 | 49.2 |

### Example 17

Synthesis of Linker Reagent 17f

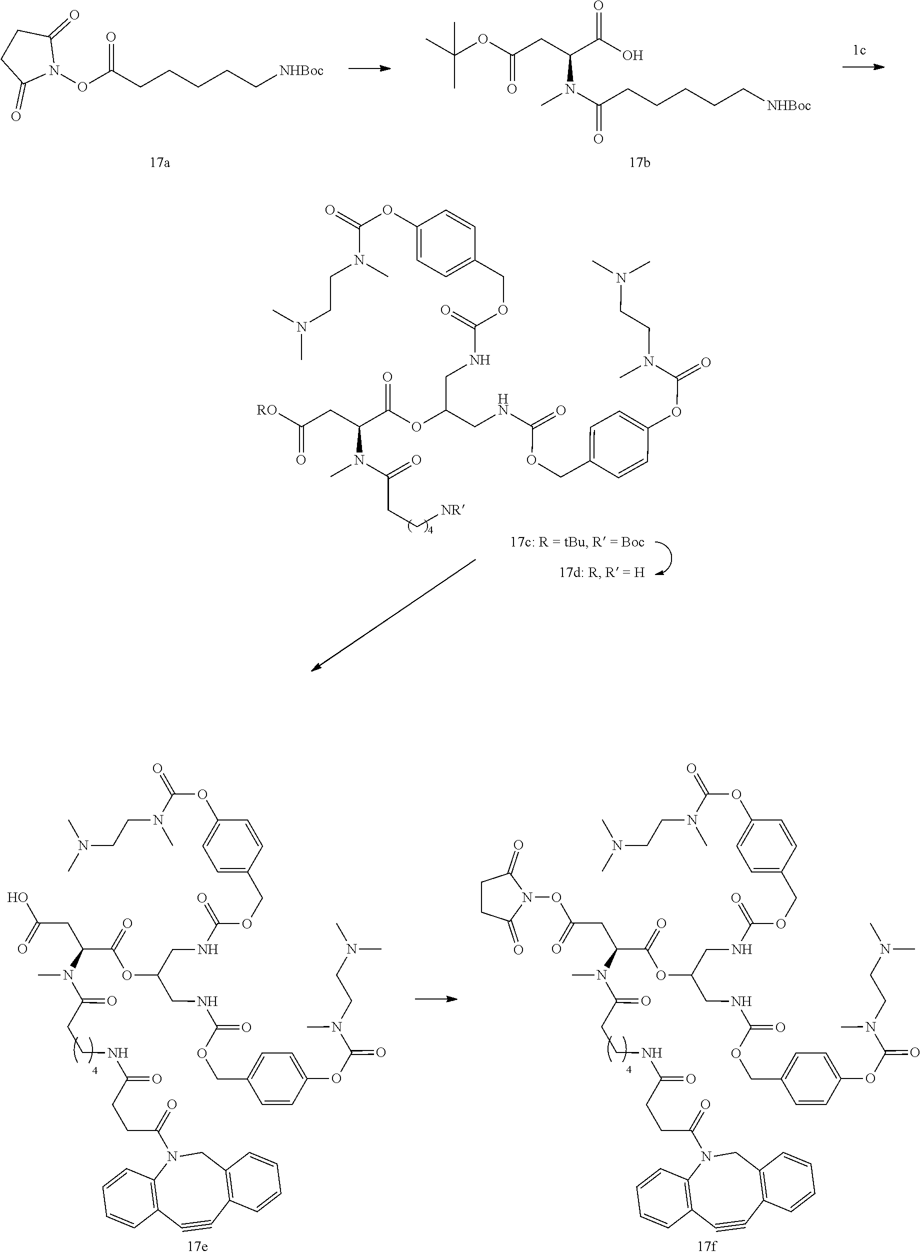

17a (880 mg, 2.68 mmol) was dissolved in DCM (4 mL) and added to a suspension of N-Me-L-Asp(tBu)-OH (519 mg, 2.55 mmol) in DCM (3 mL) and DIPEA (892 μL, 5.10 mmol). DCM (2 mL) and DMF (1.5 mL) were added to the colorless suspension and it was warmed to 40° C. giving a colorless solution.

Acetic acid (438 μL, 7.66 mmol) was added after 7 h 30 min. The solvent was evaporated, and the product purified by RP-LPLC yielding 17b.

Yield: 480 mg (45%)

MS: m/z 417.25=[M+H]⁺, (calculated=417.51).

17b (480 mg, 1.15 mmol) and 1c (760 mg, 0.87 mmol) were dissolved in acetonitrile (20 mL). DMAP (212 mg, 1.74 mmol) was added under stirring and to the resulting solution DIC (541 μL, 3.48 mmol) was added. After 1 h and 55 min TFA (332 μL, 4.35 mmol) was added and the solvent was removed in vacuo. The product was purified by RP-LPLC yielding 17c.

Yield: 810 mg (73%, 2×TFA salt)

MS: m/z 1045.74=[M+H]⁺, (calculated=1045.57).

17c (810 mg, 0.64 mmol) was dissolved in DCM (3.5 mL). TFA (3.5 mL, 45.7 mmol) was added under stirring. After 2 h 15 min the solvent was evaporated, and the residue was co-evaporated with 25 mL of DCM. The residue was dried under high vacuum overnight yielding 945 mg of 17d, which was used without further purification. 17d (56 mg, 45.2 μmol) was dissolved in acetonitrile (500 μL) and DBCO-C4-NHS ester (20 mg, 49.7 μmol) was added.

DIPEA (79 μL, 0.452 mmol) was added. After 45 min the reaction was quenched by addition of TFA (35 μL, 0.452 mmol). Water (400 μL) was added and the product purified by preparative HPLC yielding 17e.

Yield: 54 mg (84% over 2 steps, 2×TFA salt)

MS: m/z 1176.77=[M+H]⁺, (calculated=1176.55).

17e (35 mg, 24.9 μmol) was dissolved in acetonitrile (500 μL) and EDC (14 mg, 74.8 μmol) and HOSu (8.6 mg, 74.8 μmol) were added. After 4 h 15 min the reaction was quenched by addition of TFA (1.9 μL, 24.9 μmol). The reaction mixture was diluted with water (500 μL) and the product was purified by preparative-HPLC yielding 17f.

Yield: 31.6 mg (84%, 2×TFA salt)

MS: m/z 1274.86=[M+H]⁺, (calculated=1273.57).

### Example 18: Preparation of PEG Based Amino Hydrogels

PEG based amino hydrogels were synthesized as described in example 3 of WO2011/012715A1 using different crosslinking degrees to give different levels of amine content. All crosslinkers were based on 3.3 kDa PEG and were synthesized as described in example 2 of WO2011/012715A1 using azelaic acid. The hydrogels were characterized by their free amine content: HG-1: 0.191 mmol/g, HG-3: 0.215 mmol/g.

### Example 19: Preparation of PEG Based Amino Hydrogel with Free Azides HG-2

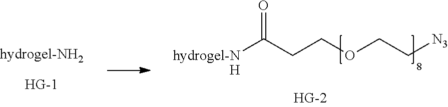

HG-1 (50 mg, 9.55 μmol) was placed into a 5 mL fritted syringe, swollen in 3 mL 1% DIPEA in NMP and washed with 1% DIPEA in NMP (10×3 mL). Azido-PEG8-NHS ester (16.2 mg, 28.7 μmol) was dissolved in 1% DIPEA in NMP, added to the hydrogel and the syringe was shaken overnight at room temperature. After 2 h 45 min, shaking was stopped, and the hydrogel was washed with NMP (10×3 mL). HG-2 was suspended in 1.1 mL NMP, transferred into an Eppendorf tube and the suspension was used without further characterization.

### Example 20: Preparation of Ubiquitin-Linker Conjugate Mixture 20b

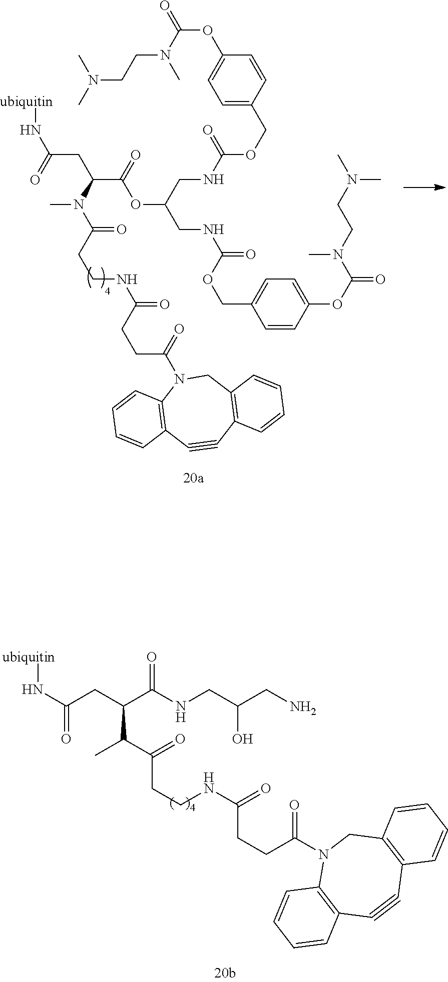

Ubiquitin from bovine erythrocytes (50 mg, 5.86 μmol) was dissolved in pH 7.4 60 mM sodium phosphate buffer (3.84 mL) and a solution of 17f in DMSO (87.9 μl, 0.1 M, 8.79 μmol) was added. The reaction was incubated for 10 min at rt and 653 μl pH 7.4 60 mM phosphate buffer containing 200 mM TriMED were added to the reaction to facilitate cleavage of the protecting groups and rearrangement of the ester 20a to the amide 20b. The reaction was mixed and incubated for 23 h 20 min at 25° C. The turbid reaction mixture was filtered through a PES membrane filter. To remove unconjugated linker from the protein conjugate mixture a buffer exchange to PBST pH 7.4 was performed using an Äkta pure system equipped with GE HiTrap columns (3 in a row) at a flow rate of 1.5 ml/min. 10.5 ml of the product fraction were collected which contains ubiquitin and ubiquitin linker conjugates 20b. 6.5 mL of the product fraction were concentrated to 4 mL using an Amicon Ultra-15 centrifugal filter unit (MWCO: 3 kDa). The concentrated solution with an estimated protein concentration of 0.89 mM was directly used for loading into hydrogel HG-2.

### Example 21: Preparation of Ubiquitin-Linker-Hydrogel Conjugate 21

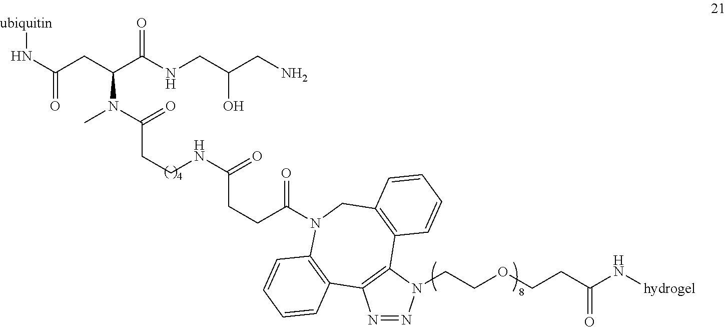

Conjugation of ubiquitin/ubiquitin-linker conjugate mixture 20b to azido functionalized, crosslinked PEG hydrogel HG-2 was performed by addition of solution 20b to hydrogel HG-2. 75 μL of hydrogel suspension HG-2 were transferred into a 5 mL fritted syringe and washed with water (10×2 mL) and PBST-buffer pH 7.4 (10×2 mL). The hydrogel was incubated with 4 mL of protein/protein-linker conjugate solution 20b and shaken for 18 h 30 min at room temperature. The hydrogel was washed with PBST-buffer pH 7.4 (10×2 mL) and transferred into a 1.5 mL Eppendorf tube yielding a 7.2 wt % solution in PBST-buffer pH 7.4.

### Example 22: In Vitro Release Kinetics for 21

40 μL of suspension 21 (corresponding to approximately 0.74 mg ubiquitin) were transferred into a sterile, 1.5 mL Eppendorf tube. 1 mL 60 mM sodium phosphate, 3 mM EDTA, pH 7.4 buffer was added to the tube, which was subsequently mixed and incubated at 37° C. in a water bath. After different time intervals, a small sample of the supernatant was analyzed by HPLC at 215 nm and the released ubiquitin was quantified in comparison to a ubiquitin standard. The obtained values were corrected for the ubiquitin removal from previous sampling times.

Release from 21:

| | Ubiquitin | Ubiquitin |
|---|---|---|
| t [d] | release [μg] | release [%] |
| 1.93 | 25 | 3.4 |
| 6.01 | 74 | 10.0 |
| 19.93 | 214 | 29.0 |
| 26.78 | 283 | 38.3 |
| 34.82 | 337 | 45.5 |
| 41.98 | 370 | 50.0 |
| 48.95 | 387 | 52.3 |
| 61.93 | 430 | 58.2 |

### Example 23: Synthesis of MTS-PEG12-NHS Ester 16c

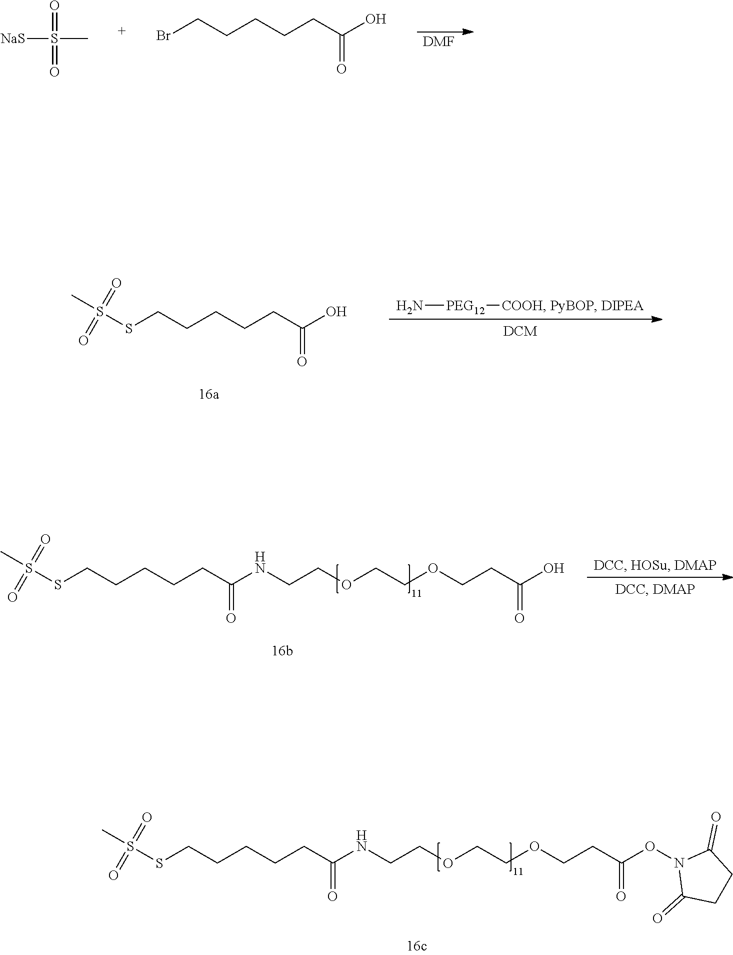

6-Bromohexanoic acid (5.89 g, 30.2 mmol, 1.0 eq.) and sodium methanethio-sulfonate (4.05 g, 30.2 mmol, 1.0 eq.) were dissolved in anhydrous DMF (47.1 mL) under argon atmosphere and stirred at 80° C. for three hours. After cooling to r.t., the mixture was diluted with water (116 mL) and extracted with diethyl ether (3×233 mL). The combined organic layers were washed with brine (350 mL), dried over MgSO₄, filtered and concentrated under reduced pressure to a volume of 40 mL. The solution was split and added to two portions of cold n-heptane (2×1150 mL) and the mixtures were cooled to −18° C. overnight. The supernatant solutions were decanted and the precipitates were dissolved in diethylether (80 mL combined). This solution was split and added to two portions of cold n-heptane (2×1000 mL) and the mixtures were cooled to −18° C. for two hours. The precipitate was collected by filtration and dried in high vacuum overnight to yield intermediate 16a (5.62 g, 24.8 mmol, 82%).

MS: m/z 249.02=[M+Na]⁺, (calculated monoisotopic mass: [M]=226.03)

DIPEA (2.76 mL, 15.9 mmol, 3.28 eq.) was added to a stirring solution of 16a (1.15 g, 5.08 mmol, 1.05 eq.) and PyBOP (2.64 g, 5.08 mmol, 1.05 eq.) in anhydrous DCM (54.8 mL). After stirring for 30 minutes, 1-amino-3,6,9,12,15,18,21,24,27,30,33,36-dodecaoxanonatriacontan-39-oic acid (2.99 g, 4.84 mmol, 1.00 eq.) was added and the mixture was stirred at room temperature for additional 30 minutes. Cold MTBE (55 mL) was added to the slightly yellow reaction mixture and it was cooled to −20° C. overnight. No precipitate was formed. All volatiles were removed in vacuo and the residue was dissolved in DCM. After addition of TFA (1.2 mL), the solution was concentrated to 10 mL. Cold MTBE (55 mL) was added to the slightly yellow solution and it was cooled to −20° C. overnight. The supernatant was decanted and the yellow precipitate was washed with cold MTBE (55 mL). The now white residue was dried on the rotavapor. After further purification by preparative RP-HPLC, intermediate 16b (2.81 g, 3.40 mmol, 70%) was obtained as white solid.

MS: m/z 826.35=[M+H]⁺, (calculated monoisotopic mass: [M]=825.39)

16b (2.81 g, 3.40 mmol, 1.0 eq.), HOSu (470 mg, 4.08 mmol, 1.2 eq.), DMAP (41.6 mg, 0.34 mmol; 0.1 eq.) and DCC (842 mg, 4.08 mmol, 1.2 eq.) were dissolved in anhydrous DCM (32.6 mL) and the mixture was stirred at room temperature for 30 minutes. The precipitate was removed by filtration and the solvent was evaporated from the filtrate. The residue was purified by preparative RP-HPLC to yield pure reagent 16c (1.74 g; 1.88 mmol, 55%).

MS: m/z 923.45=[M+H]⁺, (calculated monoisotopic mass: [M]=922.40)

### Example 24: Synthesis of MTS-Functionalized Hydrogel HG-4

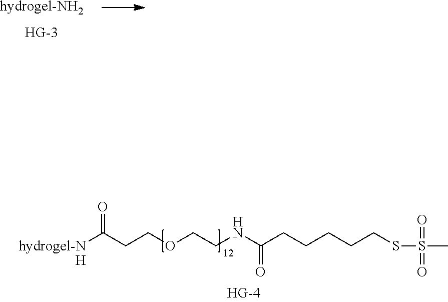

PEG-hydrogel HG-3 (500 mg, amine content: 0.215 mmol/g, 1.0 eq.), present as a suspension in a mixture of NMP/n-propylamine (99:1 v/v) was partitioned between five 20 mL syringe reactors with PE frits in equal aliquots. Each hydrogel portion was successively washed with anhydrous NMP (10×8 mL), NMP/DIPEA (99:1 v/v, 6×8 mL) and all solvents were expelled completely after complete washing. To each hydrogel portion, an aliquot of 5 mL of a freshly prepared solution of 16c (309 mg, 3.0 eq.) in anhydrous NMP (22 mL) and NMP/DIPEA (99:1 v/v, 3 mL) were drawn. The syringe reactors were agitated at 500 rpm for 180 minutes. The reaction mixtures were expelled from all syringes and each hydrogel portion was successively washed with anhydrous NMP (10×8 mL), water containing 0.1% AcOH and 0.01% Tween 20 (10×8 mL) and 20 mM succinate 0.01% Tween 20 pH 4.0 buffer (10×8 mL). The hydrogel aliquots were combined in a 50 mL Falcon tube with additional 20 mM succinate 0.01% Tween 20 pH 4.0 buffer. After brief centrifugation, the volume of the suspension was adjusted to 24 mL by removing an adequate volume of the clear supernatant to yield a suspension of MTS-hydrogel HG-4 in 20 mM succinate 0.01% Tween 20 pH 4.0 buffer with 24 mL volume and a hydrogel content of 23.7 mg/mL. The MTS load for dry hydrogel was determined as 0.189 mmol/g.

### Example 25: Preparation of HHC^{MET}-Linker Conjugate Mixture 31

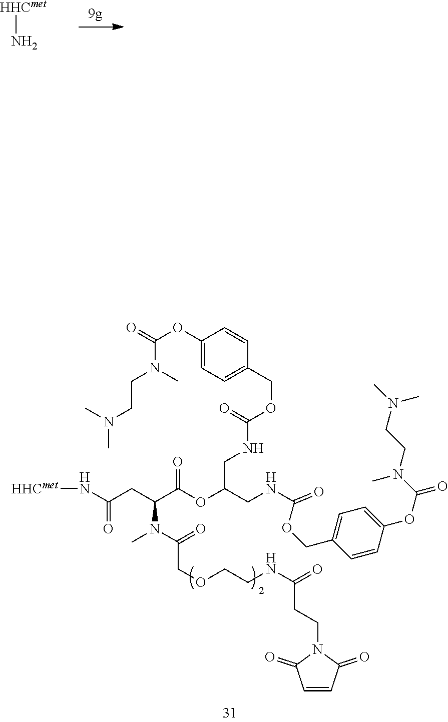

154 mL of HHC^{MET} at 5.94 mg/mL in PBS, pH 7.4 was used in this example. HHC^{MET} was concentrated using Amicon Ultra 15, MWCO 3 kDa (Merck), and the protein concentration was determined. 28.18 mL HHC^{MET} in PBS, pH 7.4 at a concentration of 30.3 mg/mL were prepared. 1.5 mol eq. (508 μL) of linker reagent 9g (corrected with respect to NHS content, 100 mM stock solution in DMSO) relative to the amount of HHC^{MET} were added to the protein solution. The reaction mixture was mixed carefully and incubated for 5 min at ambient temperature yielding a mixture of unmodified HHC^{MET} and the protected HHC^{MET} conjugates (e.g. monoconjugate, bioconjugate) 31.

The linker-conjugation reaction was immediately followed by a pH shift towards about pH 4 and a buffer exchange was performed to remove excess linker species from the HHC^{MET}/HHC^{MET}-linker conjugate mixture 31. The buffer shift was achieved by addition of 0.047 vol. eq. (1.324 mL) of 0.4 M succinic acid, pH 3.0 with respect to the volume of the HHC^{MET} solution (28.18 mL), and the solution was mixed carefully end-over-end. The buffer exchange to 20 mM succinic acid, pH 4.0 was performed using an Äkta purifier 100 system equipped with a GE HiPrep column at a flow rate of 8.0 mL/min. Six runs with approx. 5 mL injection volume per run were performed.

To determine the content of protected HHC^{MET}-linker mono-, bis-, and tris-conjugate within HHC^{MET}/protected HHC^{MET}-linker conjugate mixture 31, a PEGylation with 20 kDa PEG thiol and subsequent SE-HPLC analysis was performed. 24.4 μL unmodified HHC^{MET}/protected HHC^{MET}-linker conjugate mixture 31 (c=10.89 mg/mL) were pH-adjusted to pH 5.5 by addition of 0.154 vol. eq. (3.8 μL) of 0.5 M succinic acid, pH 6.2 with respect to the volume of the HHC^{MET} solution (24.4 μL). The obtained solution was then supplemented with 1/19 vol. eq. 20 mM succinic acid, 100 mM EDTA, 0.2% Tween20, pH 5.5 (1.5 μL) with respect to the volume of 28.2 μL. The protein concentration of the unmodified HHC^{MET}/protected HHC^{MET}-linker conjugate mixture 31 at pH 5.5 was adjusted to 4 mg/mL by mixing 15.8 μL of the solution with 20.2 μL of 20 mM succinic acid, 5 mM EDTA, 0.010% Tween20, pH 5.5. The PEGylation reaction was started by the addition of 4 μL of 15 mM PEG20-SH solution in water. After 15 minutes incubation at ambient temperature, SE-HPLC was performed using an Agilent 1200 system connected to a Superdex 200 Increase 10/300 GL column with PBS-T, pH 7.4 as mobile phase. Maleimide content was calculated with the use of the peak area of the conjugates and multiplied with the number of attached PEG reagents. A total maleimide content of 47.7% was determined for the HHC^{MET}/protected HHC^{MET}-linker conjugate mixture 31.

After analysis and overnight storage at 4° C., 71.98 mL of the HHC^{MET}/protected HHC^{MET}-linker conjugate mixture 31 (c=10.89 mg/mL) were pH-adjusted to pH 5.5 by addition of 0.154 vol. eq. (11.08 mL) of 0.5 M succinic acid pH 6.2. The obtained solution was supplemented with 1/19 vol. eq. 20 mM succinic acid, 100 mM EDTA, 0.2% Tween20, pH 5.5 (4.37 mL) and the solution was mixed end-over-end. The sample was filtered using one qpore Plastic vacuum filter (PVDF membrane) with a pore size of 0.22 μm.

### Example 26: Synthesis of Transient HHC^{MET}-Linker-Hydrogel Prodrug 32

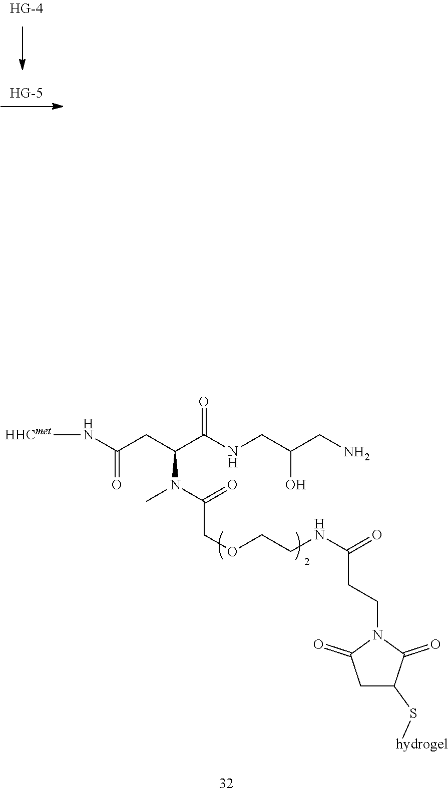

Conjugation of HHC^{MET}/HHC^{MET}-linker conjugate mixture 31 to the reduced thiol functionalized hydrogel HG-5 was performed by addition of HHC^{MET}/HHC^{MET}-linker conjugate mixture 31 to 1.75 mol. eq. of thiol groups in hydrogel HG-5 with respect to determined total maleimide content of 47.7% (19.13 μmol) in the HHC^{MET}/HHC^{MET}-linker conjugate mixture 31.

8.5 mL of MTS functionalized hydrogel HG-4 (23.7 mg/mL nominal gel content with a thiol content of 0.183 mmol/g) in 20 mM succinic acid, 0.01% Tween20, pH 4.0 were transferred into a 20 mL syringe with a frit. The thiol functionalized hydrogel was reduced by replacement of the storage solution by 20 mL of 50 mM TCEP solution in PBS-T and incubation for 15 minutes at ambient temperature. Afterwards, the 50 mM TCEP solution was removed from the syringe, and the hydrogel was washed in the syringe 10 times with 20 mL 20 mM succinic acid, 5 mM EDTA, 0.01% Tween20, pH 5.5 and resuspended in ˜6.7 mL of 20 mM succinic acid, 5 mM EDTA, 0.01% Tween20, pH 5.5 to yield HG-5.

3.06 mL of hydrogel HG-5 were transferred into two 50 mL falcon tubes. Afterwards, 43.2 mL of the HHC^{MET}/protected HHC^{MET}-linker conjugate mixture 31 (c=9.26 mg/mL) at pH 5.5 were added into each falcon tube containing hydrogel HG-5. The resulting suspensions were mixed well and incubated at ambient temperature under gentle rotation overnight yielding protected transient HHC^{MET}-linker hydrogel prodrug.

After overnight incubation, the protected transient HHC^{MET}-linker hydrogel prodrug was transferred into a 20 mL syringe equipped with a frit, and washed in the syringe once with 20 mL 20 mM succinic acid, 5 mM EDTA, 0.01% Tween20, pH 5.5 and two times with 20 mL 10 mM iodoacetamide in 30 mM sodium phosphate, 50 mM TriMED, 0.01% Tween20, pH 7.4. The protected transient HHC^{MET}-linker hydrogel prodrug was incubated for 60 minutes with gentle rotation in 30 mM sodium phosphate, 10 mM iodoacetamide, 50 mM TriMED, 0.01% Tween20, pH 7.4 buffer in the syringe at ambient temperature. After, the hydrogel was washed ten times in the syringe with 20 mL 30 mM sodium phosphate, 200 mM TriMED, 0.01% Tween20, pH 7.4. The solvent was each time discarded.

20 mL 30 mM sodium phosphate, 200 mM TriMED, 0.01% Tween20, pH 7.4 buffer were drawn up into the syringe and the resulting suspension was incubated at 25° C. for 26 hours under gentle rotation yielding transient HHC^{MET}-linker hydrogel prodrug 32. Formulation of transient HHC^{MET}-linker hydrogel prodrug 32 was achieved by washing the hydrogel ten times in the syringe with 20 mL 20 mM succinic acid, 8.5% α-α-D-trehalose, 1% carboxymethylcellulose, 0.010% Tween20, pH 5.0.

### Example 27: In Vitro Release Kinetics for 32

25 μL each of suspension 32 (corresponding to approximately 0.76 mg HHC^{MET}) were transferred into 16 sterile, 1.5 mL Eppendorf tubes. 60 mM Sodium phosphate, 3 mM EDTA, 0.01% Tween-20, pH=7.40 was added to the tubes (filled up to 0.5 ml or 1 ml). The samples were subsequently mixed and incubated at 37° C. in a water bath. After different time intervals two samples were sacrificed and the supernatant was analyzed for protein concentration using absorbance at 280 nm. The protein release over time was fitted using curve-fitting software to obtain a half-life of 27 d.

### Example 28: Plasma Pharmacokinetics of HHC^{MET} in Wistar Rats after Subcutaneous (SC) and Intramuscular (IM) Injections of a Transient HHC^{MET}-Linker Hydrogel Prodrug 32 and after Intravenous (IV) and Subcutaneous (SC) Injections of Free HHC^{MET}

This study was performed in order to investigate the plasma pharmacokinetics of HHC^{MET} in Wistar rats following subcutaneous and intramuscular administration of transient HHC^{MET}-linker hydrogel prodrug 32 or following subcutaneous or intravenous administration of free HHC^{MET}. Animals (n=3 per group) received either a single SC injection in the neck region or a single IM injection in the thigh musculature of a formulation of 32 (10 mg/kg HHC^{MET} equivalents) or a single SC injection in the neck region or IV injection in the tail vein of an HHC^{MET} formulation (10 mg/kg HHC^{MET}). At selected time points, 200 μL blood were collected in Li-Heparin tubes and processed to plasma by centrifugation at 3,000 g for 10 minutes at 4° C. HHC^{MET} concentrations in rat plasma were determined with an in-house developed sandwich ELISA setup. For capturing HHC^{MET}, a human CTLA-4 (AA Ala37-Ser160)-Fc Tag fusion protein (Supplier AcroBiosystem, Newark, Del.; USA, catalog no. CT4-H5255) was coated to the ELISA plate wells and read-out was performed via a rabbit anti-camelid VHH antibody conjugated with horseradish peroxidase (supplier Genscript, Piscataway, N.J., USA, catalog no. A01861-200).

Calibration standards of HHC^{MET} in blank plasma were prepared as follows: thawed Li-Heparin Wistar rat plasma was homogenized. The free HHC^{MET} formulation was spiked into blank plasma at concentrations between 96.0 ng/mL and 3.00 ng/mL with additional higher and lower anchor points. These solutions were used for the generation of a calibration curve. Calibration curves were analyzed via a 4-parameter logarithmic fit and 1/Y weighted. Calibration curves were confirmed via separately prepared quality control standards at 10, 40 and 80 ng/mL.

The determined HHC^{MET} plasma concentrations are depicted in Table 3.

TABLE 3
Determined mean HHC^{MET} concentrations in
ng/mL per time point and group (n = 3).

| | Time (h) | | | | | | | | |
|---|---|---|---|---|---|---|---|---|---|
| Group | 0.25 | 1 | 4 | 24 | 32 | 48 | 72 | 96 | 168 |
| 1 | — | 5.40 | 30.2 | 52.9 | 39.7 | 41.2 | 33.7 | 34.8 | 38.7 |
| 2 | 2540 | 5090 | 2280 | 2.60 | <LLOQ | <LLOQ | 2.07 | <LLOQ | <LLOQ |
| 3 | 17400 | 3530 | 1720 | 8.27 | <LLOQ | <LLOQ | 1.15 | <LLOQ | <LLOQ |
| 4 | — | 5.09 | 27.5 | 55.3 | 54.4 | 46.2 | 37.1 | 38.9 | 56.4 |

Group 1: Transient HHC^{MET}-linker hydrogel prodrug 32 (10 mg/kg HHC^{MET} equivalents - subcutaneous administration);
Group 2: HHC^{MET} (10 mg/kg - subcutaneous administration);
Group 3: HHC^{MET} (10 mg/kg - intravenous administration);
Group 4: Transient HHC^{MET}-linker hydrogel prodrug 32 (10 mg/kg HHC^{MET} equivalents - intramuscular administration);
method LLOQ at 3.00 ng/mL;
,,—” denotes sample not taken

Specifically, HHC^{MET} concentration 72 h after intra-tissue (subcutaneous or intramuscular) injection of transient HHC^{MET}-linker hydrogel prodrug 32 (10 mg/kg HHC^{MET} equivalents) is at least 80% of HHC^{MET} concentration 1 h after intra-tissue (subcutaneous or intramuscular) injection of transient HHC^{MET}-linker hydrogel prodrug 32 (10 mg/kg HHC^{MET} equivalents).

### Abbreviations

A alanine

Ac acetyl

Ala alanine

aq. aqueous

Asp aspartate

Bzl benzyl

Boc tert-butyloxycarbonyl

Cbz benzyloxycarbonyl

DCC N,N′-dicyclohexylcarbodiimide

DBCO dibenzoazacyclooctyne

DBU 1,8-diazabicyclo (5.4.0)undec-7-ene

DCM dichloromethane

DIC N,N′-diisopropylcarbodiimide

DIPEA diisopropylethylamine

DMAP dimethylaminopyridine

DMF dimethylformamide

DMSO dimethylsulfoxide

Dpr 2,3-diaminopropionic acid

DS degree of substitution

DTNB 5,5′-dithiobis-(2-nitrobenzoic acid)

eq equivalent

EDC 1-ethyl-3-(3-dimethylaminopropyl)carbodiimide hydrochloric acid salt

EDTA ethylenediaminetetraacetic acid

F phenylalanine

Fmoc fluorenylmethyloxycarbonyl

HA hyaluronic acid

HG hydrogel

HEPES 4-(2-hydroxyethyl)piperazine-1-ethanesulfonic acid

HFIP 1,1,1,3,3,3-hexafluoroisopropanol

HOBt 1-hydroxybenzotriazole

HOSu N-hydroxysuccinimide

HPLC high performance liquid chromatography

IAA iodo acetamide

ivDDe 1-(4,4-dimethyl-2,6-dioxocyclohexylidene)-3-methylbutyl

K lysine

LC liquid chromatography

LCMS liquid chromatography mass spectrometry

LLOQ lower limit of quantification

LPLC low pressure liquid chromatography

Lys lysine

mAB monoclonal antibody

MeOH methanol

Me methyl

MES 4-morpholineethanesulfonic acid

MTBE methyl tert-butyl ether

MTS methanethiosulfonyl

MWCO molecular weight cut-off

NHS N-hydroxysuccinimide

NMP N-methyl-2-pyrrolidone

OPA o-phthalaldehyde

PBS phosphate-buffered saline pH 7.4

PBST phosphate-buffered saline pH 7.4 with Tween-20

PE polyethylene

PEG polyethylene glycol

PES polyethersulfone

Phe phenylalanine

PTFE polytetrafluoroethylene

PVDF polyvinylidenefluoride

PyBOP benzotriazol-1-yl-oxytripyrrolidinophosphonium hexafluorophosphate

RP reversed phase

rt/r.t. room temperature

sat. saturated

SE size exclusion

Ser serine

SPDP N-succinimidyl 3-(2-pyridyldithio)propionate

Su succinimide

tBu and t-Bu tert-butyl

TCEP tris(2-carboxyethyl)phosphine hydrochloride

TFA trifluoroacetic acid

THF tetrahydrofuran

TriMED N,N,N′-trimethylethylene diamine

TSTU O—(N-succinimidyl)-N,N,N′,N′-tetramethyluronium tetrafluoroborate

UPLC ultra performance liquid chromatography

1. A conjugate or a pharmaceutically acceptable salt thereof comprising at least one moiety -D conjugated via at least one moiety -L¹-L²- to at least one moiety Z, wherein a moiety -L¹- is conjugated to the nitrogen of a primary or secondary amine of a moiety -D and wherein the linkage between -D and -L¹- is reversible and wherein a moiety -L²- is conjugated to Z, wherein
each -D is independently a primary or secondary amine-comprising moiety of a drug D-H;
each -L²- is independently a single bond or a spacer moiety;
each Z is independently a polymeric moiety or a C₈₋₂₄ alkyl;
each -L¹- is independently a linker moiety of formula (I): 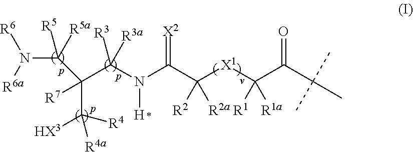
wherein
the dashed line indicates the attachment to the nitrogen of the primary or secondary amine of -D;
v is selected from the group consisting of 0 or 1;
—X¹— is selected from the group consisting of —C(R⁸)(R^{8a})—, —N(R⁹)— and —O—;
═X² is selected from the group consisting of ═O and ═N(R¹⁰);
—X³— is selected from the group consisting of —O—, —S— and —Se—;
each p is independently selected from the group consisting of 0 or 1, provided that at most one p is 0;
—R⁶, —R^{6a}, —R¹⁰ are independently selected from the group consisting of —H, —C(R¹¹)(R^{11a})(R^{11b}) and -T;
—R⁹ is selected from the group consisting of —C(R¹¹)(R^{11a})(R^{11b}) and -T;
—R¹, —R^{1a}, —R², —R^{2a}, —R³, —R^{3a}, —R⁴, —R^{4a}, —R⁵, —R^{5a}, —R⁷, —R⁸ —R^{8a}, —R¹¹, —R^{1a} and —R^{11b} are independently selected from the group consisting of —H, halogen, —CN, —C(O)OR 1, —OR 1, —C(O)R¹², —C(O)N(R¹²)(R^{12a}), —S(O)₂N(R¹²)(R^{12a}), —S(O)N(R¹²)(R^{12a}), —S(O)₂R¹², —S(O)R¹², —N(R¹²)S(O)₂N(R^{12a})(R^{12b}), —SR¹², —NO₂, —N(R¹²)C(O)OR^{12a}, —N(R¹²)C(O)N(R^{12a})(R^{12b}), —OC(O)N(R¹²)(R^{12a}), -T, C₁₋₆ alkyl, C₂₋₆ alkenyl and C₂₋₆ alkynyl; wherein C₁₋₆ alkyl, C₂₋₆ alkenyl and C₂₋₆ alkynyl are optionally substituted with one or more —R¹³, which are the same or different; and wherein C₁₋₆ alkyl, C₂₋₆ alkenyl and C₂₋₆ alkynyl are optionally interrupted by one or more groups selected from the group consisting of -T-, —C(O)O—, —O—, —C(O)—, —C(O)N(R¹⁴)—, —S(O)₂N(R¹⁴)—, —S(O)N(R¹⁴)—, —S(O)₂—, —S(O)—, —N(R¹⁴)S(O)₂N(R^{14a})—, —S—, —N(R¹⁴)—, —OC(OR¹⁴)(R^{14a})—, —N(R¹⁴)C(O)N(R^{14a})— and —OC(O)N(R¹⁴)—;
—R¹², —R^{12a}, —R^{12b} are independently selected from the group consisting of —H, -T, C₁₋₆ alkyl, C₂₋₆ alkenyl and C₂₋₆ alkynyl; wherein -T, C₁₋₆ alkyl, C₂₋₆ alkenyl and C₂₋₆ alkynyl are optionally substituted with one or more —R¹³, which are the same or different and wherein C₁₋₆ alkyl, C₂₋₆ alkenyl and C₂₋₆ alkynyl are optionally interrupted by one or more groups selected from the group consisting of -T-, —C(O)O—, —O—, —C(O)—, —C(O)N(R¹⁴)—, —S(O)₂N(R¹⁴)—, —S(O)N(R¹⁴)—, —S(O)₂—, —S(O)—, —N(R¹⁴)S(O)₂N(R^{14a})—, —S—, —N(R¹⁴)—, —OC(OR¹⁴)(R^{14a})—, —N(R¹⁴)C(O)N(R^{14a})— and —OC(O)N(R¹⁴)—;
wherein each T is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl; wherein each T is independently optionally substituted with one or more —R¹³, which are the same or different;
—R¹³ is selected from the group consisting of halogen, —CN, oxo, —C(O)OR¹⁵, —OR¹⁵, —C(O)R¹⁵, —C(O)N(R¹⁵)(R^{15a}), —S(O)₂N(R¹⁵)(R^{15a}), —S(O)N(R¹⁵)(R^{15a}), —S(O)₂R¹⁵, —S(O)R¹⁵, —N(R¹⁵)S(O)₂N(R^{15a})(R^{15b}), —SR¹⁵, —N(R¹⁵)(R^{15a}), —NO₂, —OC(O)R¹⁵, —N(R¹⁵)C(O)R^{15a}, —N(R¹⁵)S(O)₂R^{15a}, —N(R¹⁵)S(O)R^{15a}, —N(R¹⁵)C(O)OR^{15a}, —N(R¹⁵)C(O)N(R^{15a})(R^{15b}), —OC(O)N(R¹⁵)(R^{15a}) and C₁₋₆ alkyl; wherein C₁₋₆ alkyl is optionally substituted with one or more halogen, which are the same or different;
wherein —R¹⁴, —R^{14a}, —R¹⁵, —R^{15a} and —R^{15b} are independently selected from the group consisting of —H and C₁₋₆ alkyl; wherein C₁₋₆ alkyl is optionally substituted with one or more halogen, which are the same or different;
optionally, one or more of the pairs —R/—R^{1a}, —R²/—R^{2a}, —R³/—R^{3a}, —R⁴/—R^{4a}, —R⁵/—R^{5a} or —R⁵/—R^{a} are joined together with the atom to which they are attached to form a C₃₋₁₀ cycloalkyl, 3- to 10-membered heterocyclyl or an 8- to 11-membered heterobicyclyl;
optionally, one or more of the pairs —R¹/—R², —R¹/—R′, —R¹/—R⁹, —R²/—R⁹ or —R²/—R¹⁰ are joined together with the atoms to which they are attached to form a ring -A-;
wherein -A- is selected from the group consisting of phenyl, naphthyl, indenyl, indanyl, tetralinyl, C₃₋₁₀ cycloalkyl, 3- to 10-membered heterocyclyl and 8- to 11-membered heterobicyclyl;
optionally, one or more of the pairs —R³/—R⁶, —R⁴/—R⁶, —R⁵/—R⁶, —R⁶/—R^{6a} or —R⁶/—R⁷ can form together with the atoms to which they are attached a ring -A′-;
wherein -A′- is selected from the group consisting of 3- to 10-membered heterocyclyl and 8- to 11-membered heterobicyclyl; and
each -L¹- is substituted with at least one -L²- and optionally further substituted provided that the hydrogen marked with the asterisk in formula (I) is not replaced by a substituent.

2. The conjugate or pharmaceutically acceptable salt thereof of claim 1, wherein -D is selected from the group consisting of small molecule, medium size molecule, oligonucleotide, peptide nucleic acid, peptide and protein drug moieties.

3. The conjugate or pharmaceutically acceptable salt thereof of claim 1 or 2, wherein -D is selected from the group consisting of small molecule, medium size, peptide and protein drug moieties.

4. The conjugate or pharmaceutically acceptable salt thereof of any one of claims 1 to 3, wherein -D is a protein drug moiety.

5. The conjugate or pharmaceutically acceptable salt thereof of any one of claims 1 to 4, wherein v is 0.

6. The conjugate or pharmaceutically acceptable salt thereof of any one of claims 1 to 5, wherein ═X² is ═O.

7. The conjugate or pharmaceutically acceptable salt thereof of any one of claims 1 to 6, wherein —X³— is —O—.

8. The conjugate or pharmaceutically acceptable salt thereof of any one of claims 1 to 7, wherein both —R³ and —R^{3a} are —H.

9. The conjugate or pharmaceutically acceptable salt thereof of any one of claims 1 to 8, wherein -L¹- is of formula (I-a): 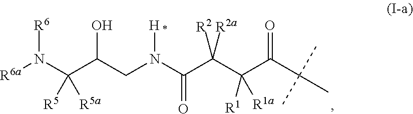
wherein
the dashed line indicates the attachment to the nitrogen of the primary or secondary amine of -D of claim 1; and
—R¹, —R^{1a}, —R², —R^{2a}, —R⁵, —R^{5a}, —R⁶, —R^{6a}, -L²- and Z are used as defined in claim 1.

10. The conjugate or pharmaceutically acceptable salt thereof of any one of claims 1 to 9, wherein both —R⁶ and —R^{6a} are —H.

11. The conjugate or pharmaceutically acceptable salt thereof of any one of claims 1 to 10, wherein both —R¹ and —R^{1a} are —H.

12. The conjugate or pharmaceutically acceptable salt thereof of any one of claims 1 to 11, wherein -L¹- is of formula (I-d): 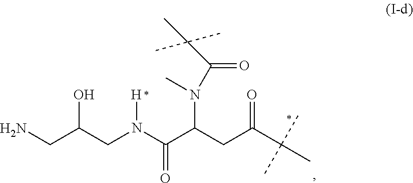
wherein
the dashed line marked with the asterisk indicates the attachment to the nitrogen of the primary or secondary amine of -D of claim 1 and the unmarked dashed line indicates attachment to -L²-; and wherein -L²- and Z are used as defined in claim 1.

13. The conjugate or pharmaceutically acceptable salt thereof of any one of claims 1 to 12, wherein Z is a polymeric moiety.

14. The conjugate or pharmaceutically acceptable salt thereof of any one of claims 1 to 13, wherein Z is a water-insoluble polymeric moiety.

15. The conjugate or pharmaceutically acceptable salt thereof of any one of claims 1 to 14, wherein Z is a water-insoluble polymeric moiety comprising a polymer selected from the group consisting of 2-methacryloyl-oxyethyl phosphoyl cholins, poly(acrylic acids), poly(acrylates), poly(acrylamides), poly(alkyloxy) polymers, poly(amides), poly(amidoamines), poly(amino acids), poly(anhydrides), poly(aspartamides), poly(butyric acids), poly(glycolic acids), polybutylene terephthalates, poly(caprolactones), poly(carbonates), poly(cyanoacrylates), poly(dimethylacrylamides), poly(esters), poly(ethylenes), poly(ethyleneglycols), poly(ethylene oxides), poly(ethyl phosphates), poly(ethyloxazolines), poly(glycolic acids), poly(hydroxyethyl acrylates), poly(hydroxyethyl-oxazolines), poly(hydroxymethacrylates), poly(hydroxypropylmethacrylamides), poly(hydroxypropyl methacrylates), poly(hydroxypropyloxazolines), poly(iminocarbonates), poly(lactic acids), poly(lactic-co-glycolic acids), poly(methacrylamides), poly(methacrylates), poly(methyloxazolines), poly(organophosphazenes), poly(ortho esters), poly(oxazolines), poly(propylene glycols), poly(siloxanes), poly(urethanes), poly(vinyl alcohols), poly(vinyl amines), poly(vinylmethylethers), poly(vinylpyrrolidones), silicones, celluloses, carbomethyl celluloses, hydroxypropyl methylcelluloses, chitins, chitosans, dextrans, dextrins, gelatins, hyaluronic acids and derivatives, functionalized hyaluronic acids, mannans, pectins, rhamnogalacturonans, starches, hydroxyalkyl starches, hydroxyethyl starches and other carbohydrate-based polymers, xylans, and copolymers thereof.

16. The conjugate or pharmaceutically acceptable salt thereof of any one of claims 1 to 13, wherein Z is a water-soluble polymeric moiety.

17. The conjugate or pharmaceutically acceptable salt thereof of any one of claims 1 to 16, wherein -L²- is a spacer moiety selected from the group consisting of -T′-, —C(O)O—, —O—, —C(O)—, —C(O)N(R^{y1})—, —S(O)₂N(R^{y1})—, —S(O)N(R^{y1})—, —S(O)₂—, —S(O)—, —N(R^{y1})S(O)₂N(R^{y1a})—, —S—, —N(R^{y1})—, —OC(OR^{y1})(R^{y1a})—, —N(R^{y1})C(O)N(R^{y1a})—, —OC(O)N(R^{y1})—, C₁₋₅₀ alkyl, C₂₋₅₀ alkenyl, and C₂₋₅₀ alkynyl; wherein -T′-, C₁₋₅₀ alkyl, C₂₋₅₀ alkenyl and C₂₋₅₀ alkynyl are optionally substituted with one or more —R^{y2}, which are the same or different and wherein C₁₋₅₀ alkyl, C₂₋₅₀ alkenyl, and C₂₋₅₀ alkynyl are optionally interrupted by one or more groups selected from the group consisting of -T′-, —C(O)O—, —O—, —C(O)—, —C(O)N(R^{y3})—, —S(O)₂N(R^{y3})—, —S(O)N(R^{y3})—, —S(O)₂—, —S(O)—, —N(R^{y3})S(O)₂N(R^{y3a})—, —S—, —N(R^{y3})—, —OC(OR^{y3})(R^{y3a})—, —N(R^{y3})C(O)N(R^{y3a})—, and —OC(O)N(R^{y3})—;
wherein —R^{y1} and —R^{y1a} are independently selected from the group consisting of —H, -T′, C₁₋₅₀ alkyl, C₂₋₅₀ alkenyl and C₂₋₅₀ alkynyl; wherein -T′, C₁₋₅₀ alkyl, C₂₋₅₀ alkenyl, and C₂₋₅₀ alkynyl are optionally substituted with one or more —R^{y2}, which are the same or different, and wherein C₁₋₅₀ alkyl, C₂₋₅₀ alkenyl and C₂₋₅₀ alkynyl are optionally interrupted by one or more groups selected from the group consisting of -T′-, —C(O)O—, —O—, —C(O)—, —C(O)N(R^{y4})—, —S(O)₂N(R^{y4})—, —S(O)N(R^{y4})—, —S(O)₂—, —S(O)—, —N(R^{y4})S(O)₂N(R^{y4a})—, —S—, —N(R^{y4})—, —OC(OR^{y4})(R^{y4a})—, —N(R^{y4})C(O)N(R^{y4a})—, and —OC(O)N(R^{y4})—;
each T′ is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl and 8- to 30-membered heteropolycyclyl; wherein each T′ is independently optionally substituted with one or more —R^{y2}, which are the same or different;
each —R^{y2} is independently selected from the group consisting of halogen, —CN, oxo (═O), —COOR^{y5}, —OR^{y5}, —C(O)R^{y5}, —C(O)N(R^{y5}R^{y5a}), —S(O)₂N(R^{y5}R^{y5a}), —S(O)N(R^{y5}R^{y5a}), —S(O)₂R^{y5}, —S(O)R^{y5}, —N(R^{y5})S(O)₂N(R^{y5a}R^{y5b}), —SR^{y5}, —N(R^{y5}R^{y5a}), —NO₂, —OC(O)R^{y5}, —N(R^{y5})C(O)R^{y5a}, —N(R^{y5})S(O)₂R^{y5a}, —N(R^{y5})S(O)R^{y5a}, —N(R^{y5})C(O)OR^{y5a}, —N(R^{y5})C(O)N(R^{y5a}R^{y5b}), —OC(O)N(R^{y5}R^{y5a}), and C₁₋₆ alkyl; wherein C₁₋₆ alkyl is optionally substituted with one or more halogen, which are the same or different; and
each —R^{y3}, —R^{y3}a, —R^{y4}, —R^{y4}a, —R^{y5}, —R^{y5a} and —R^{y5b} is independently selected from the group consisting of —H and C₁₋₆ alkyl; wherein C₁₋₆ alkyl is optionally substituted with one or more halogen, which are the same or different.

18. The conjugate or pharmaceutically acceptable salt thereof of any one of claims 1 to 17, wherein -L²- has a molecular weight ranging from 14 g/mol to 750 g/mol.

19. A reagent comprising a moiety -L*-, wherein the moiety -L*- is conjugated to -Q, wherein
-Q is —OH or -LG, wherein -LG is a leaving group moiety;
-L*- is a linker moiety of formula (II): 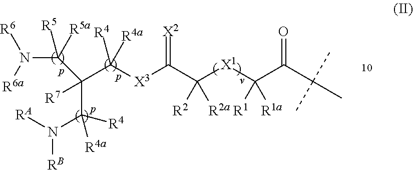
wherein
the dashed line indicates the attachment to -Q;
v is selected from the group consisting of 0 or 1;
—X¹— is selected from the group consisting of —C(R⁸)(R^{8a})—, —N(R⁹)— and —O—;
═X² is selected from the group consisting of ═O and ═N(R¹⁰);
—X³— is selected from the group consisting of —O—, —S— and —Se—;
each p is independently selected from the group consisting of 0 or 1, provided that at most one p is 0;
—R⁶ is —PG and —R^{6a} is selected from the group consisting of —H, —C(R¹¹)(R^{11a})(R^{11b}), -T and —PG; or —R⁶ and —R^{6a} are independently selected from the group consisting of —C(R¹¹)(R^{11a})(R^{11b}) and -T;
—R^{A} and —R^{B} are independently selected from the group consisting of —H and —PG provided that not more than one of —R^{A} or —RB can be —H;
—PG is an amine protecting group moiety;
—R⁹ is selected from the group consisting of —C(R¹)(R^{11a})(R^{11b}) and -T;
—R¹⁰ is selected from the group consisting of H, —C(R¹¹)(R^{11a})(R^{11b}) and -T;
—R¹, —R^{1a}, —R², —R^{2a}, —R³, —R^{3a}, —R⁴, —R^{4a}, —R⁵, —R^{5a}, —R⁷, —R⁸ —R^{8a}, —R¹¹, —R^{11a} and —R^{11b} are independently selected from the group consisting of —H, halogen, —CN, —C(O)OR¹², —OR¹², —C(O)R¹², —C(O)N(R)(R^{12a}), —S(O)₂N(R¹²)(R^{12a}), —S(O)N(R¹²)(R^{12a}), —S(O)₂R¹², —S(O)R¹², —N(R¹²)S(O)₂N(R^{12a})(R^{12b}), —SR¹², —NO₂, —N(R¹²)C(O)OR^{12a}, —N(R¹²)C(O)N(R^{12a})(R^{12b}), —OC(O)N(R¹²)(R^{12a}), -T, C₁₋₆ alkyl, C₂₋₆ alkenyl and C₂₋₆ alkynyl; wherein C₁₋₆ alkyl, C₂₋₆ alkenyl and C₂₋₆ alkynyl are optionally substituted with one or more —R¹³, which are the same or different; and wherein C₁₋₆ alkyl, C₂₋₆ alkenyl and C₂₋₆ alkynyl are optionally interrupted by one or more groups selected from the group consisting of -T-, —C(O)O—, —O—, —C(O)—, —C(O)N(R¹⁴)—, —S(O)₂N(R¹⁴)—, —S(O)N(R¹⁴)—, —S(O)₂—, —S(O)—, —N(R¹⁴)S(O)₂N(R^{14a})—, —S—, —N(R¹⁴)—, —OC(OR¹⁴)(R^{14a})—, —N(R¹⁴)C(O)N(R^{14a})— and —OC(O)N(R¹⁴)—; —R¹², —R^{12a}, —R^{12b} are independently selected from the group consisting of —H, -T, C₁₋₆ alkyl, C₂₋₆ alkenyl, and C₂₋₆ alkynyl; wherein -T, C₁₋₆ alkyl, C₂₋₆ alkenyl, and C₂₋₆ alkynyl are optionally substituted with one or more —R¹³ which are the same or different and wherein C₁₋₆ alkyl, C₂₋₆ alkenyl, and C₂₋₆ alkynyl are optionally interrupted by one or more groups selected from the group consisting of -T-, —C(O)O—, —O—, —C(O)—, —C(O)N(R¹⁴)—, —S(O)₂N(R¹⁴)—, —S(O)N(R¹⁴)—, —S(O)₂—, —S(O)—, —N(R¹⁴)S(O)₂N(R^{14a})—, —S—, —N(R¹⁴)—, —OC(OR¹⁴)(R^{14a})—, —N(R¹⁴)C(O)N(R^{14a})— and —OC(O)N(R¹⁴)—;
wherein each T is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl;
wherein each T is independently optionally substituted with one or more —R¹³, which are the same or different;
—R¹³ is selected from the group consisting of halogen, —CN, oxo, —C(O)OR¹⁵, —OR¹⁵, —C(O)R¹⁵, —C(O)N(R¹⁵)(R^{15a}), —S(O)₂N(R¹⁵)(R^{15a}), —S(O)N(R¹⁵)(R^{15a}), —S(O)₂R¹⁵, —S(O)R¹⁵, —N(R¹⁵)S(O)₂N(R^{15a})(R^{15b}), —SR¹⁵, —N(R¹⁵)(R^{15a}), —NO₂, —OC(O)R¹⁵, —N(R¹⁵)C(O)R^{15a}, —N(R¹⁵)S(O)₂R^{15a}, —N(R¹⁵)S(O)R^{15a}, —N(R¹⁵)C(O)OR^{15a}, —N(R¹⁵)C(O)N(R^{15a})(R^{15b}), —OC(O)N(R¹⁵)(R^{15a}) and C₁₋₆ alkyl; wherein C₁₋₆ alkyl is optionally substituted with one or more halogen, which are the same or different;
wherein —R¹⁴, —R^{14a}, —R¹⁵, —R^{15a} and —R^{15b} are independently selected from the group consisting of —H and C₁₋₆ alkyl; wherein C₁₋₆ alkyl is optionally substituted with one or more halogen, which are the same or different;
optionally, one or more of the pairs —R⁶/—R^{6a}, —R^{A}/—R^{B} or —R⁶/—R^{A} can form a moiety —PG;
optionally, one or more of the pairs —R¹/—R^{1a}, —R²/—R^{2a}, —R³/—R^{3a}, —R⁴/—R^{4a}, —R⁵/—R^{5a} or —R⁵/—R^{a} are joined together with the atom to which they are attached to form a C₃₋₁₀ cycloalkyl, 3- to 10-membered heterocyclyl or an 8- to 11-membered heterobicyclyl;
optionally, one or more of the pairs —R¹/—R², —R¹/—R′, —R¹/—R⁹, —R²/—R⁹ or —R²/—R¹⁰ are joined together with the atoms to which they are attached to form a ring -A-;
wherein -A- is selected from the group consisting of phenyl, naphthyl, indenyl, indanyl, tetralinyl, C₃₋₁₀ cycloalkyl, 3- to 10-membered heterocyclyl and 8- to 11-membered heterobicyclyl;
optionally, one or more of the pairs —R³/—R⁶, —R⁴/—R⁶, —R⁵/—R⁶, —R⁶/—R^{6a} or —R⁶/—R⁷ can form together with the atoms to which they are attached a ring -A′-;
wherein -A′- is selected from the group consisting of 3- to 10-membered heterocyclyl and 8- to 11-membered heterobicyclyl;
wherein -L*- is optionally substituted with at least one moiety -L²-Z or at least one moiety -L²-Y and optionally is further substituted;
wherein -L²- is a single bond or a spacer moiety;
Z is independently a polymeric moiety or a C₈₋₂₄ alkyl;
and wherein —Y is a functional group which may optionally be present in its protected form.

20. The reagent of claim 19, wherein -L*- of formula (II) is substituted with at least one moiety -L²-Y or at least one moiety -L²-Z and optionally is further substituted.

21. The reagent of claim 19 or 20, wherein -L*- of formula (II) is substituted with one moiety -L²-Y.

22. The reagent of any one of claims 19 to 21, wherein —Y is selected from the group consisting of thiol, maleimide, amine, hydroxyl, carboxylic acid and derivatives, carbonate and derivatives, carbamate and derivatives, isothiocyanate, disulfide, pyridyl disulfide, methylthiosulfonyl, vinylsulfone, aldehyde, ketone, haloacetyl, selenide, azide, —NH—NH₂, —O—NH₂, a terminal alkyne, a compound of formula (z′i) 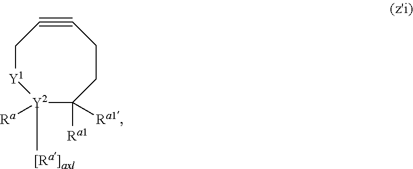
wherein
Y¹, Y² are independently C or N,
R^{a}, R^{a′}, R^{a1}, R^{a1′} are independently —H or C₁₋₆ alkyl,
ax1 is 0, if Y² is N; ax1 is 1, if Y² is C,
optionally, the pair R^{a}/R^{a1} forms a chemical bond, if Y² is C,
optionally, the pair R^{a′}/R^{a1′} are joined together with the atom to which they are attached to form a ring A′, if Y² is C, and
A′ is cyclopropyl or phenyl;
a compound of formula (z′ii) 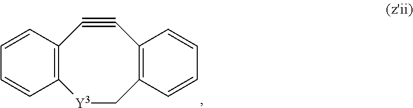
wherein
Y³ is C or N;
a compound of formula (z′iii) 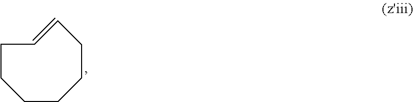
a compound of formula (z′iv), 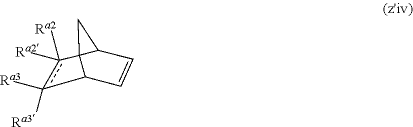
wherein
R^{a2}, R^{a2′}, R^{a3}, R^{a3′} are —H, [symbol]
indicates a single or double bond,
optionally, the pair R^{a2′}/R^{a3′} are joined together with the atoms to which they are attached to form a ring A^{1′}; and
A^{1′} is 5-membered heterocyclyl;
a compound of formula (z′v) 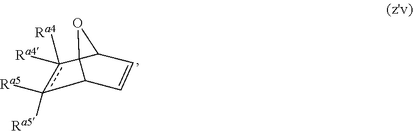
wherein
R^{a4}, R^{a4′}, R^{a5}, R^{a5′} are —H, [symbol]
indicates a single or double bond,
optionally, the pair R^{a4}/R^{a5} forms a chemical bond,
optionally, the pair R^{a4′}/R^{a5′} are joined together with the atoms to which they are attached to form a ring A^{2′}, and
A^{2′} is 5-membered heterocyclyl;
a compound of formula (z′vi) 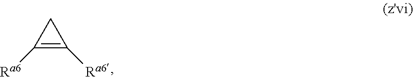
wherein
R^{a6}, R^{a6′} are either both C₁₋₆ alkyl or one of R^{a6}, R^{a6′} is —H and the other one is selected from C₁₋₆ alkyl, —COOR^{a7}, —CONHR^{a7′}, and CH₂OR^{a7″}, and
R^{a7}, R^{a7′}, R^{a7″} are independently —H or C₁₋₄ alkyl;
a compound of formula (z′vii) 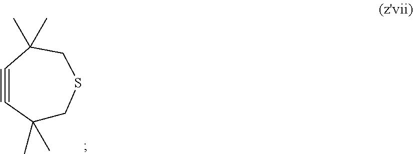
a compound of formula (z′viii) 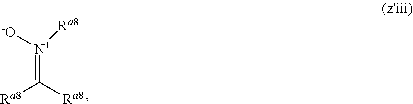
wherein
R^{a8}, R^{a8′}, R^{a8″} are independently selected from the group consisting of —H and C₁₋₄ alkyl;
a compound of formula (z′ix) 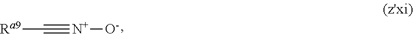
wherein
R^{a9} is —H or C₁₋₄ alkyl;
a compound of formula (z′x) 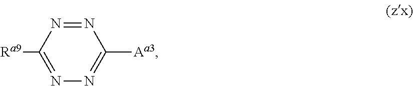
wherein
R^{a9} is selected from —COOR^{a11}, —CONHR^{a11}, and 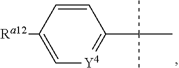
wherein
Y⁴ is C or N,
R^{a12} is selected from the group consisting of —H, —COOR^{a13}, —CONR^{a13}R^{a13′}, —CH₂NR^{a13}R^{a13′}, and —NR^{a13}COR^{a13′}, and
R^{a13}, R^{a13′} are independently selected from the group consisting of —H and C₁₋₄ alkyl,
A^{a3} is selected from the group consisting of —H, methyl, tert-butyl, —CF₃, —COOR, 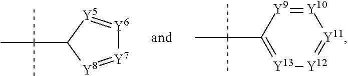
wherein
each Y⁵, Y⁶, Y⁷, Y⁸ is independently C or N, provided that no more than 3 of Y⁵, Y⁶, Y⁷, Y⁸ are N,
each of Y⁹, Y¹, Y¹¹, Y¹², Y¹³ is either C, N, S or O, provided that no more than 4 of Y⁹, Y¹⁰, Y¹¹, Y¹², Y¹³ are N, S, or O;
a compound of formula (z′xi) 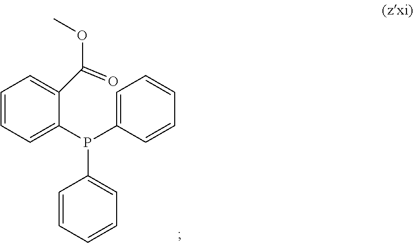 [SMILES: COC(=O)c1ccccc1P(c1ccccc1)c1ccccc1]
a compound of formula (z′xii) 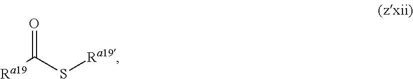
wherein
R^{a19}, R^{a19′} are independently selected from the group consisting of —H, C₁₋₆ alkyl, C₂₋₆ alkenyl, C₂₋₆ alkynyl, C₃₋₈ cycloalkyl, 3- to 10-membered heterocyclyl, 8- to 11-membered heterobicyclyl, phenyl, naphthyl, indenyl, indanyl, and tetralinyl;
a compound of formula (z′xiii) 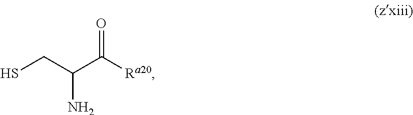
wherein
R^{a20} is selected from the group consisting of —H, C₁₋₆ alkyl, C₂₋₆ alkenyl, C₂₋₆ alkynyl, C₃₋₈ cycloalkyl, 3- to 10-membered heterocyclyl, 8- to 11-membered heterobicyclyl, phenyl, naphthyl, indenyl, indanyl, and tetralinyl;
a compound of formula (z′xiv)
R^{a22}—Ar—Y¹⁴ (z′xiv),
wherein
Ar is selected from phenyl, naphthyl, indenyl, indanyl, and tetralinyl,
Y¹⁴ is halogen,
R^{a22}, R^{a23}, R^{a23′} are independently selected from the group consisting of —H, C₁₋₆ alkyl, C₂₋₆ alkenyl, C₂₋₆ alkynyl, C₃₋₈ cycloalkyl, 3- to 10-membered heterocyclyl, 8- to 11-membered heterobicyclyl, phenyl, naphthyl, indenyl, indanyl, and tetralinyl;
a compound of formula (z′xv) 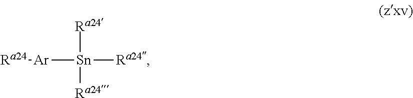
Ar is selected from phenyl, naphthyl, indenyl, indanyl, and tetralinyl,
R^{a24}, R^{a24′}, R^{a24″}, R^{a24′″} are independently selected from the group consisting of —H, C₁₋₆ alkyl, C₂₋₆ alkenyl, C₂₋₆ alkynyl, C₃₋₈ cycloalkyl, 3- to 10-membered heterocyclyl, 8- to 11-membered heterobicyclyl, phenyl, naphthyl, indenyl, indanyl, and tetralinyl;
a compound of formula (z′xvi) 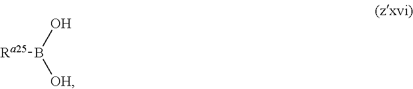
wherein
R^{a25} is selected from the group consisting of —H, C₁₋₆ alkyl, C₂₋₆ alkenyl, C₂₋₆ alkynyl, C₃₋₈ cycloalkyl, 3- to 10-membered heterocyclyl, 8- to 11-membered heterobicyclyl, phenyl, naphthyl, indenyl, indanyl, and tetralinyl;
a compound of formula (z′xvii) 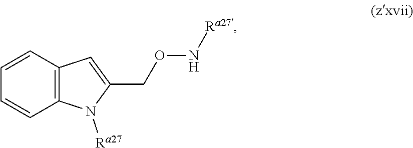
wherein
R^{a27}, R^{a27′} are independently —H or C₁₋₆ alkyl;
a compound of formula (z′xviii) 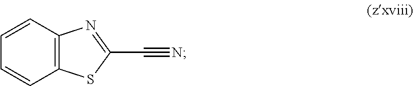 [SMILES: N#Cc1nc2ccccc2s1]
a compound of formula (z′xix)
R^{a12}—PPh₂ (z′xix),
wherein
—PPh₂ represents a group having the following formula 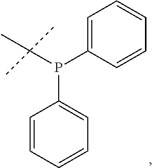
wherein the dashed line indicates attachment to the rest of the moiety of formula (z′xix),
R^{a12} is selected from the group consisting of 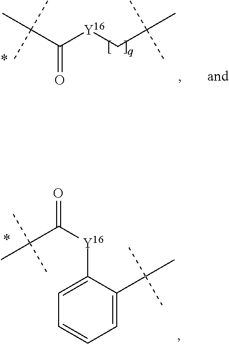
wherein
the unmarked dashed line indicates attachment to the rest of the moiety of formula (z′xix),
the dashed line with the asterisk indicates attachment to -L²-,
q is 1 or 2, and
Y¹⁶ is O or S;
a compound of formula (z′xx) 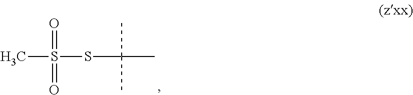
wherein the dashed line indicates attachment to -L²-; and
a compound of formula (z′xxi) 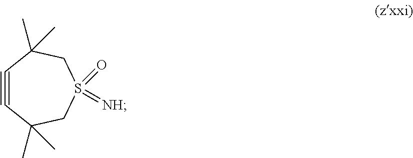
wherein the moieties of formula (z′i), (z′ii), (z′iii), (z′iv), (z′v), (z′vi), (z′vii), (z′viii), (z′ix), (z′x), (z′xi), (z′xii), (z′xiii), (z′xiv), (z′xv), (z′xvi), (z′xvii), (z′xviii) and (z′xxi) are substituted with a moiety -L²- and are optionally further substituted.

23. The reagent of any one of claims 19 to 22, wherein -Q is -LG.

24. The reagent of any one of claims 19 to 23, wherein —R⁶ is —PG and —R^{6a} is —H.

25. The reagent of any one of claims 19 to 24, wherein —X³— is —O—.

26. The reagent of any one of claims 19 to 25, wherein -L*- is of formula (II′): 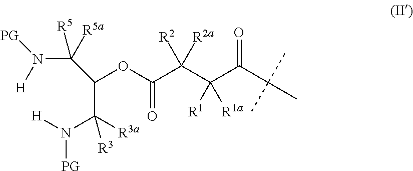
wherein the dashed line indicates attachment to -Q;
—R¹, —R^{1a}, —R², —R^{2a}, —R³, —R^{3a}, —R⁵, —R^{5a} and —PG are used as defined in claim 12;
-L*- is substituted with at least one moiety -L²-Z or at least one moiety -L²-Y and optionally is further substituted; and
wherein -L²-, —Y and Z are used as defined in claim 19.

27. The reagent of any one of claims 19 to 26, wherein —R¹, —R^{1a}, —R², —R^{2a}, —R³, —R^{3a}, —R⁵ and —R^{8a} are —H.

28. An intermediate (A) comprising a moiety -L*- of formula (II) of any one of claims 19 to 27, wherein -L*- is conjugated to at least one moiety -D, wherein
each -D is independently a primary or secondary amine-comprising moiety of a drug D-H;
the dashed line in formula (II) indicates the attachment to the nitrogen of the primary or secondary amine of -D;
-L*- of formula (II) is optionally substituted with at least one moiety -L²-Z or at least one moiety -L²-Y and optionally is further substituted;
-L²- is independently a single bond or a spacer moiety;
Z is independently a polymeric moiety or a C₈₋₂₄ alkyl;
and wherein —Y is a functional group which may optionally be present in its protected form.

29. The intermediate of claim 28, wherein -L*- of formula (II) is substituted with at least one moiety -L²-Y or at least one moiety -L²-Z and optionally is further substituted;

30. A method for synthesizing a conjugate of any one of claims 1 to 18, wherein the method comprises the steps of:
(a) providing a reagent comprising a linker -L*- of formula (II) of any one of claims 19 to 27;
(b) conjugating the reagent of step (a) with a primary or secondary amine-comprising drug to obtain an intermediate (A);
(c) subjecting the intermediate (A) of step (b) to deprotection conditions to obtain an intermediate (C′) or conjugate comprising a linker -L¹- of formula (I) or an intermediate (B);
(d) optionally, subjecting the intermediate (B) or (C′) obtained from step (c) to shift conditions;
(e) optionally, deprotecting the intermediate (B) or (C′) of step (d); and
(f) isolating the conjugate resulting from steps (c), (d) or (e);
wherein optionally at least one Z moiety is attached to at least one intermediate (A) (B) or (C′) in between steps (b) and (c), (c) and (d), (d) and (e) or (e) and (f).

31. A pharmaceutical composition comprising the conjugate or pharmaceutically acceptable salt thereof of any one of claims 1 to 18.

32. The conjugate or pharmaceutically acceptable salt thereof of any one of claims 1 to 18 or the pharmaceutical composition of claim 31 for use as a medicament.

33. The conjugate or pharmaceutically acceptable salt thereof of any one of claims 1 to 18 or the pharmaceutical composition of claim 31 for use in a method of treating a disease that can be treated with D-H.

34. A method of preventing a disease or treating a patient suffering from a disease that can be prevented or treated with D-H, comprising administering an effective amount of the conjugate or the pharmaceutically acceptable salt thereof of any one of claims 1 to 18 or the pharmaceutical composition of claim 31 to the patient.
